SEQUENCE LISTING (88 sequences)

SEQ ID NO: 1 - protein, 195 aa, Artificial Sequence
  Description of Artificial Sequence Synthetic polypeptide
PEICKQDIVFDGIAQIRGEIFFFKDRFIWRTVTPRDKPMGPLLVATFWPELPEKIDAVYEAPQEEKAVFFAGNEYWIYSASTLERGYPKPLTSLGLPPDVQRVDAAFNWSKNKKTYIFAGDKFWRYNEVKKKMDPGFPKLIADAWNAIPDNLDAVVDLQGGGHSYFFKGAYYLKLENQSLKSVKFGSIKSDWLGC

SEQ ID NO: 2 - protein, 195 aa, Gallus gallus
PELCKHDIVFDGVAQIRGEIFFFKDRFMWRTVNPRGKPTGPLLVATFWPDLPEKIDAVYESPQDEKAVFFAGNEYWVYTASNLDRGYPKKLTSLGLPPDVQRIDAAFNWGRNKKTYIFSGDRYWKYNEEKKKMELATPKFIADSWNGVPDNLDAVLGLTDSGYTYFFKDQYYLQMEDKSLKIVKIGKISSDWLGC

SEQ ID NO: 3 - protein, 305 aa, Mus musculus
MAAREESTQSANRVLRISQLDALELNKALEQLVWSQFTQCFHGFKPGLLARFEPEVKAFLWLFLWRFTIYSKNATVGQSVLNIQHKNDSSPNPVYQPPSKNQKLLYAVCTIGGRWLEERCYDLFRNRHLASFGKAKQCMNFVVGLLKLGELMNFLIFLQKGKFATLTERLLGIHSVFCKPQNMREVGFEYMNRELLWHGFAEFLIFLLPLINIQKLKAKLSSWCTLCTGAAGHDSTLGSSGKECALCGEWPTMPHTIGCEHVFCYYCVKSSFLFDIYFTCPKCGTEVHSVQPLKAGIQMSEVNAL

SEQ ID NO: 4 - protein, 305 aa, Rattus norvegicus
MAAREESTQSANRVLRISQLDALELNKALEQLVWSQFTQCFHGFKPGLLARFEPEVKAFLWLFLWRFTIYSKNATVGQSVLNIQYKNDSSPNPVYQPPSKNQKLLYAVCTIGGRWLEERCYDLFRNRHLASFGKAKQCMNFVVGLLKLGELMNFLIFLQKGKFATLTERLLGIHSVFCKPQSMREVGFEYMNRELLWHGFAEFLVFLLPLINIQKLKAKLSSWCIPLTSTAGSDSTLGSSGKECALCGEWPTMPHTIGCEHVFCYYCVKSSFLFDMYFTCPKCGTEVHSVQPLKSGIEMSEVNAL

SEQ ID NO: 5 - protein, 195 aa, Oryctolagus cuniculus
PEICTQDIVFDGIAQIRGEIFFFKDRFIWRTVTPGDKPMGPLLVATFWPELPEKIDAVYEAPQEEKAVFFAGNEYWVYSASTLERGYPKPLTSLGLPPDVQRVDAAFNWSKNKKTYIFAGDKFWRYNEVKKKMDPGFPRLIADAWNAIPDHLDAVVDLQGSGHSYFFKGTYYLKLENQSLKSVKVGSIKTDWLGC

SEQ ID NO: 6 - protein, 195 aa, Bos taurus
PEVCDSKLTFDAITTIRGEVMFFKDRFYMRTNPLYPEVELNFISVFWPQLPNGLQAAYEVADRDEVRFFKGNKYWAVKGQDVLRGYPRDIYRSFGFPRTVKSIDAAVSEEDTGKTYFFVANKCWRYDEYKQSMDAGYPKMIAEDFPGIGNKVDAVFQKGGFFYFFHGRRQYKFDPQTKRILTLLKANSWFNCRKN

SEQ ID NO: 7 - protein, 195 aa, Equus caballus
PEVCDSKLTFDAITTIRGEVMFFKDRFYMRINPYYPEAELNFISIFWPQLPNGLQAAYEVSHRDEVRFFKGNKYWAVKGQDVLYGYPKDIHRSFGFPSTVKNIDAAVSEEDTGKTYFFVADKYWRYDEYKRSMDAGYPKMIADDFPGIGDKVDAVFQKDGFFYFFHGTRQYKFDPKTKRILTLQKANSWFNCRKN

SEQ ID NO: 8 - protein, 195 aa, Homo sapiens
PKACDSKLTFDAITTIRGEVMFFKDRFYMRTNPFYPEVELNFISVFWPQLPNGLEAAYEFADRDEVRFFKGNKYWAVQGQNVLHGYPKDIYSSFGFPRTVKHIDAALSEENTGKTYFFVANKYWRYDEYKRSMDPGYPKMIAHDFPGIGHKVDAVFMKDGFFYFFHGTRQYKFDPKTKRILTLQKANSWFNCRKN

SEQ ID NO: 9 - protein, 195 aa, Sus scrofa
PQVCDSKLTFDAITTLRGELMFFKDRFYMRTNSFYPEVELNFISVFWPQVPNGLQAAYEIADRDEVRFFKGNKYWAVRGQDVLYGYPKDIHRSFGFPSTVKNIDAAVFEEDTGKTYFFVAHECWRYDEYKQSMDTGYPKMIAEEFPGIGNKVDAVFQKDGFLYFFHGTRQYQFDFKTKRILTLQKANSWFNCRKN

SEQ ID NO: 10 - protein, 195 aa, Homo sapiens; identical to SEQ ID NO: 1

SEQ ID NO: 11 - protein, 191 aa, Homo sapiens
PANCDPALSFDAVSTLRGEILIFKDRHFWRKSLRKLEPELHLISSFWPSLPSGVDAAYEVTSKDLVFIFKGNQFWAIRGNEVRAGYPRGIHTLGFPPTVRKIDAAISDKEKNKTYFFVEDKYWRFDEKRNSMEPGFPKQIAEDFPGIDSKIDAVFEEFGFFYFFTGSSQLEFDPNAKKVTHTLKSNSWLNC

SEQ ID NO: 12 - protein, 191 aa, Mus musculus
SPMCSSTLFFDAVSTLRGEVLFFKDRHFWRKSLRTPEPEFYLISSFWPSLPSNMDAAYEVTNRDTVFIFKGNQFWAIRGHEELAGYPKSIHTLGLPATVKKIDAAISNKEKRKTYFFVEDKYWRFDEKKQSMEPGFPRKIAEDFPGVDSRVDAVFEAFGFLYFFSGSSQLEFDPNAKKVTHILKSNSWFNC

SEQ ID NO: 13 - protein, 191 aa, Oryctolagus cuniculus
PVMCDPDLSFDAISTLRGEILFFKDRYFWRKSLRILEPEFHLISSFWPSLPSAVDAAYEVISRDTVFIFKGTQFWAIRGNEVQAGYPRSIHTLGFPSTIRKIDAAISDKERKKTYFFVEDKYWRFDEKRQSLEPGFPRHIAEDFPGINPKIDAVFEAFGFFYFFSGSSQSEFDPNAKKVTHVLKSNSWFQC

SEQ ID NO: 14 - protein, 191 aa, Rattus norvegicus
LPMCSSALSFDAVSTLRGEVLFFKDRHFWRKSLRTPEPGFYLISSFWPSLPSNMDAAYEVTNRDTVFILKGNQIWAIRGHEELAGYPKSIHTLGLPETVQKIDAAISLKDQKKTYFFVEDKFWRFDEKKQSMDPEFPRKIAENFPGIGTKVDAVFEAFGFLYFFSGSSQLEFDPNAGKVTHILKSNSWFNC

SEQ ID NO: 15 - protein, 192 aa, Homo sapiens
PKPCDPSLTFDAITTLRGEILFFKDRYFWRRHPQLQRVEMNFISLFWPSLPTGIQAAYEDFDRDLIFLFKGNQYWALSGYDILQGYPKDISNYGFPSSVQAIDAAVFYRSKTYFFVNDQFWRYDNQRQFMEPGYPKSISGAFPGIESKVDAVFQQEHFFHVFSGPRYYAFDLIAQRVTRVARGNKWLNCRYG

SEQ ID NO: 16 - protein, 190 aa, Mus musculus
PKACDPHLRFDATTTLRGEIYFFKEKYFWRRHPQLRTVDLNFISLFWPGLPNGLQAAYEDFDRDLVFLFKGRQYWALSGYDLQQGYPRDISNYGFPRSVQAIDAAVSYNGKTYFFINNQCWRYDNERRSMDPGYPKSIPSMFPGVNCRVDAVFLQDSFFLFFSGPQYFAFNFVSHRVTRVARSNLWLNCS

SEQ ID NO: 17 - protein, 190 aa, Rattus norvegicus
PTACDPHLRFDAATTLRGEIYFFKDKYFWRRHPQLRTVDLNFISLFWPFLPNGLQAAYEDFDRDLVFLFKGRQYWALSAYDLQQGYPRDISNYGFPRSVQAIDAAVSYNGKTYFFVNNQCWRYDNQRRSMDPGYPTSIASVFPGINCRIDAVFQQDSFFLFFSGPQYFAFNLVSRRVTRVARSNLWLNCP

SEQ ID NO: 18 - protein, 195 aa, Bos taurus
EDVCNVDIFDAIAEIRNRLHFFKAGKYWRLSEGGGRRVQGPFLVKSKWPALPRKLDSAFEDPLTKKIFFFSGRQVWVYTGASLLGPRRLDKLGLGPEVAQVTGALPRPEGKVLLFSGQSFWRFDVKTQKVDPQSVTPVDQMFPGVPISTHDIFQYQEKAYFCQDHFYWRVSSQNEVNQVDYVGYVTFDLLKCPED

SEQ ID NO: 19 - protein, 195 aa, Canis familiaris
EDICKVNIFDAIAEIRNYLHFFKEGKYWRFSKGKGRRVQGPFLSPSTWPALPRKLDSAFEDGLTKKTFFFSGRQVWVYTGTSVVGPRRLDKLGLGPEVTQVTGALPQGGGKVLLFSRQRFWSFDVKTQTVDPRSAGSVEQMYPGVPLNTHDIFQYQEKAYFCQDRFYWRVNSRNEVNQVDEVGYVTFDILQCPED

SEQ ID NO: 20 - protein, 195 aa, Homo sapiens
DDACNVNIFDAIAEIGNQLYLFKDGKYWRFSEGRGSRPQGPFLIADKWPALPRKLDSVFEEPLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 21 - protein, 200 aa, Mus musculus
DNPCNVDVFDAIAEIQGALHFFKDGWYWKFLNHRGSPLQGPFLTARTWPALPATLDSAFEDPQTKRVFFFSGRQMWVYTGKTVLGPRSLDKLGLGPEVTHVSGLLPRRPGKALLFSKGRVWRFDLKSQKVDPQSVIRVDKEFSGVPWNSHDIFQYQDKAYFCHGKFFWRVSFQNEVNKVDPEVNQVDDVGYVTYDLLQCP

SEQ ID NO: 22 - protein, 191 aa, Homo sapiens
PAKCDPALSFDAISTLRGEYLFFKDRYFWRRSHWNPEPEFHLISAFWPSLPSYLDAAYEVNSRDTVFIFKGNEFWAIRGNEVQAGYPRGIHTLGFPPTIRKIDAAVSDKEKKKTYFFAADKYWRFDENSQSMEQGFPRLIADDFPGVEPKVDAVLQAFGFFYFFSGSSQFEFDPNARMVTHILKSNSWLHC

SEQ ID NO: 23 - protein, 191 aa, Mus musculus
PDKCDPALSFDSVSTLRGEVLFFKDRYFWRRSHWNPEPEFHLISAFWPTLPSDLDAAYEAHNTDSVLIFKGSQFWAVRGNEVQAGYPKGIHTLGFPPTVKKIDAAVFEKEKKKTYFFVGDKYWRFDETRHVMDKGFPRQITDDFPGIEPQVDAVLHEFGFFYFFRGSSQFEFDPNARTVTHILKSNSWLLC

SEQ ID NO: 24 - protein, 198 aa, Homo sapiens
PDACEASFDAVSTIRGELFFFKAGFVWRLRGGQLQPGYPALASRHWQGLPSPVDAAFEDAQGHIWFFQGAQYWVYDGEKPVLGPAPLTELGLVRFPVHAALVWGPEKNKIYFFRGRDYWRFHPSTRRVDSPVPRRATDWRGVPSEIDAAFQDADGYAYFLRGRLYWKFDPVKVKALEGFPRLVGPDFFGCAEPANTFL

SEQ ID NO: 25 - protein, 198 aa, Mus musculus
PDVCETSFDAVSTIRGELFFFKAGFVWRLRSGRLQPGYPALASRHWQGLPSPVDAAFEDAQGQIWFFQGAQYWVYDGEKPVLGPAPLSKLGLQGSPVHAALVWGPEKNKIYFFRGGDYWRFHPRTQRVDNPVPRRSTDWRGVPSEIDAAFQDAEGYAYFLRGHLYWKFDPVKVKVLEGFPRPVGPDFFDCAEPANTFR

SEQ ID NO: 26 - protein, 192 aa, Homo sapiens
PALCDPNLSFDAVTTVGNKIFFFKDRFFWLKVSERPKTSVNLISSLWPTLPSGIEAAYEIEARNQVFLFKDDKYWLISNLRPEPNYPKSIHSFGFPNFVKKIDAAVFNPRFYRTYFFVDNQYWRYDERRQMMDPGYPKLITKNFQGIGPKIDAVFYSKNKYYYFFQGSNQFEYDFLLQRITKTLKSNSWFGC

SEQ ID NO: 27 - protein, 191 aa, Mus musculus
STFCHQSLSFDAVTTVGEKILFFKDWFFWWKLPGSPATNITSISSIWPSIPSAIQAAYEIESRNQLFLFKDEKYWLINNLVPEPHYPRSIYSLGFSASVKKVDAAVFDPLRQKVYFFVDKHYWRYDVRQELMDPAYPKLISTHFPGIKPKIDAVLYFKRHYYIFQGAYQLEYDPLFRRVTKTLKSTSWFGC

SEQ ID NO: 28 - protein, 191 aa, Oryctolagus cuniculus
PTACDHNLKFDAVTTVGNKIFFFKDSFFWWKIPKSSTTSVRLISSLWPTLPSGIEAAYEIGDRHQVFLFKGDKFWLISHLRLQPNYPKSIHSLGFPDFVKKIDAAVFNPSLRKTYFFVDNLYWRYDERREVMDAGYPKLITKHFPGIGPKIDAVFYFQRYYYFFQGPNQLEYDTFSSRVTKKLKSNSWFDC

SEQ ID NO: 29 - protein, 191 aa, Rattus norvegicus
STVCHQSLSFDAVTTVGDKIFFFKDWFFWWRLPGSPATNITSISSMWPTIPSGIQAAYEIGGRNQLFLFKDEKYWLINNLVPEPHYPRSIHSLGFPASVKKIDAAVFDPLRQKVYFFVDKQYWRYDVRQELMDAAYPKLISTHFPGIRPKIDAVLYFKRHYYIFQGAYQLEYDPLLDRVTKTLSSTSWFGC

SEQ ID NO: 30 - protein, 191 aa, Bos taurus
PDKCDPSLSLDAITSLRGETLIFKDRFFWRLHPQQVEAELFLTKSFGPELPNRIDAAYEHPSHDLIFIFRGRKFWALSGYDILEDYPKKISELGFPKHVKKISAALHFEDSGKTLFFSENQVWSYDDTNHVMDKDYPRLIEEVFPGIGDKVDAVYQKNGYIYFFNGPIQFEYSIWSNRIVRVMTTNSLLWC

SEQ ID NO: 31 - protein, 191 aa, Equus caballus
PDKCDPSLSLDAITSLRGETMVFKDRFFWRLHPQLVDAELFLTKSFWPELPNRIDAAYEHPSKDLIFIFRGRKFWALNGYDILEGYPQKISELGFPKDVKKISAAVHFEDTGKTLFFSGNQVWRYDDTNRMMDKDYPRLIEEDFPGIGDKVDAVYEKNGYIYFFNGPIQFEYSIWSNRIVRVMPTNSLLWC

SEQ ID NO: 32 - protein, 191 aa, Homo sapiens
PDKCDPSLSLDAITSLRGETMIFKDRFFWRLHPQQVDAELFLTKSFWPELPNRIDAAYEHPSHDLIFIFRGRKFWALNGYDILEGYPKKISELGLPKEVKKISAAVHFEDTGKTLLFSGNQVWRYDDTNHIMDKDYPRLIEEDFPGIGDKVDAVYEKNGYIYFFNGPIQFEYSIWSNRIVRVMPANSILWC

SEQ ID NO: 33 - protein, 191 aa, Oryctolagus cuniculus
PDKCDPSLSLDAITSLRGETMIFKDRFFWRLHPQQVDAELFLTKSFWPELPNRIDAAYEHPARDLIFIFRGKKFWAPNGYDILEGYPQKLSELGFPREVKKISAAVHFEDTGKTLFFSGNQVWSYDDTNHTMDQDYPRLIEEEFPGIGGKVDAVYEKNGYIYFFNGPIQFEYSIWSKRIVRVMPTNSLLWC

SEQ ID NO: 34 - protein, 196 aa, Homo sapiens
PNICDGNFDTVAMLRGEMFVFKKRWFWRVRNNQVMDGYPMPIGQFWRGLPASINTAYERKDGKFVFFKGDKHWVFDEASLEPGYPKHIKELGRGLPTDKIDAALFWMPNGKTYFFRGNKYYRFNEELRAVDSEYPKNIKVWEGIPESPRGSFMGSDEVFTYFYKGNKYWKFNNQKLKVEPGYPKSALRDWMGCPSG

SEQ ID NO: 35 - protein, 196 aa, Mus musculus
PNICDGNFDTVAMLRGEMFVFKERWFWRVRNNQVMDGYPMPIGQFWRGLPASINTAYERKDGKFVFFKGDKHWVFDEASLEPGYPKHIKELGRGLPTDKIDAALFWMPNGKTYFFRGNKYYRFNEEFRAVDSEYPKNIKVWEGIPESPRGSFMGSDEVFTYFYKGNKYWKFNNQKLKVEPGYPKSALRDWMGCPSG

SEQ ID NO: 36 - protein, 196 aa, Sus scrofa
PNICDGNFDTVAMLRGEMFVFKERWFWRVRKNQVMDGYPMPIGQFWRGLPASINTAYERKDGKFVFFKGDKHWVFDEASLEPGYPKHIKELGRRLPTDKIDAALFWMPNGKDYFFRGNKYYRFNEELRAVDSEYPKNIKVWEGIPESPRGSFMGSDEVFTYFYKGNKYWKFNNQKLKVEPGYPKSALRDWMGCPSG

SEQ ID NO: 37 - protein, 196 aa, Oryctolagus cuniculus
PKICDGNFDTVAVFRGEMFVFKERWFWRVRNNQVMDGYPMPIGQLWRGLPASINTAYERKDGKFVFFKGDKHWVFDEASLEPGYPKHIKELGRGLPTDKIDAALFWMPNGKTYFFRGNKYYRFNEELRAVDSEYPKNIKVWEGIPESPRGSFMGSDEVFTYFYKGNKYWKFNNQKLKVEPGYPKSALRDWMGCPAG

SEQ ID NO: 38 - protein, 196 aa, Rattus norvegicus; identical to SEQ ID NO: 35

SEQ ID NO: 39 - protein, 196 aa, Homo sapiens
PNICDGDFDTVAMLRGEMFVFKGRWFWRVRHNRVLDNYPMPIGHFWRGLPGDISAAYERQDGRFVFFKGDRYWLFREANLEPGYPQPLTSYGLGIPYDRIDTAIWWEPTGHTFFFQEDRYWRFNEETQRGDPGYPKPISVWQGIPASPKGAFLSNDAAYTYFYKGTKYWKFDNERLRMEPGYPKSILRDFMGCQEH

SEQ ID NO: 40 - protein, 196 aa, Mus musculus
PNICDGNFDTVAVLRGEMFVFKGRWFWRVRHNRVLDNYPMPIGHFWRGLPGNISAAYERQDGHFVFFKGNRYWLFREANLEPGYPQPLSSYGTDIPYDRIDTAIWWEPTGHTFFFQADRYWRFNEETQHGDPGYPKPISVWQGIPTSPKGAFLSNDAAYTYFYKGTKYWKFNNERLRMEPGHPKSILRDFMGCQEH

SEQ ID NO: 41 - protein, 196 aa, Homo sapiens
PNICDGNFNTLAILRREMFVFKDQWFWRVRNNRVMDGYPMQITYFWRGLPPSIDAVYENSDGNFVFFKGNKYWVFKDTTLQPGYPHDLITLGSGIPPHGIDSAIWWEDVGKTYFFKGDRYWRYSEEMKTMDPGYPKPITVWKGIPESPQGAFVHKENGFTYFYKGKEYWKFNNQILKVEPGYPRSILKDFMGCDGP

SEQ ID NO: 42 - protein, 196 aa, Mus musculus
PNICDGNFNTLAILRREMFVFKDQWFWRVRNNRVMDGYPMQITYFWRGLPPSIDAVYENSDGNFVFFKGNKYWVFKDTTLQPGYPHDLITLGNGIPPHGIDSAIWWEDVGKTYFFKGDRYWRYSEEMKTMDPGYPKPITIWKGIPESPQGAFVHKENGFTYFYKGKEYWKFNNQILKVEPGYPRSILKDFMGCDGP

SEQ ID NO: 43 - protein, 196 aa, Rattus norvegicus; identical to SEQ ID NO: 42

SEQ ID NO: 44 - protein, 198 aa, Homo sapiens
PHRCSTHFDAVAQIRGEAFFFKGKYFWRLTRDRHLVSLQPAQMHRFWRGLPLHLDSVDAVYERTSDHKIVFFKGDRYWVFKDNNVEEGYPRPVSDFSLPPGGIDAAFSWAHNDRTYFFKDQLYWRYDDHTRHMDPGYPAQSPLWRGVPSTLDDAMRWSDGASYFFRGQEYWKVLDGELEVAPGYPQSTARDWLVCGDS

SEQ ID NO: 45 - protein, 198 aa, Mus musculus
PHRCTAHFDAVAQIRGEAFFFKGKYFWRLTRDRHLVSLQPAQMHRFWRGLPLHLDSVDAVYERTSDHKIVFFKGDRYWVFKDNNVEEGYPRPVSDFSLPPGGIDAVFSWAHNDRTYFFKDQLYWRYDDHTRRMDPGYPAQGPLWRGVPSMLDDAMRWSDGASYFFRGQEYWKVLDGELEAAPGYPQSTARDWLVCGEP

SEQ ID NO: 46 - protein, 191 aa, Xenopus laevis
PSRCDPNVVFNAVTTMRGELIFFVKRFLWRKHPQASEAELMFVQAFWPSLPTNIDAAYENPITEQILVFKGSKYTALDGFDVVQGYPRNIYSLGFPKTVKRIDAAVHIEQLGKTYFFAAKKYWSYDEDKKQMDKGFPKQISNDFPGIPDKIDAAFYYRGRLYFFIGRSQFEYNINSKRIVQVLRSNSWLGC

SEQ ID NO: 47 - protein, 190 aa, Homo sapiens
PDPCSSELDAMMLGPRGKTYAFKGDYVWTVSDSGPGPLFRVSALWEGLPGNLDAAVYSPRTQWIHFFKGDKVWRYINFKMSPGFPKKLNRVEPNLDAALYWPLNQKVFLFKGSGYWQWDELARTDFSSYPKPIKGLFTGVPNQPSAAMSWQDGRVYFFKGKVYWRLNQQLRVEKGYPRNISHNWMHCRPR

SEQ ID NO: 48 - protein, 189 aa, Mus musculus
PNPCSGEVDAMVLGPRGKTYAFKGDYVWTVTDSGPGPLFQISALWEGLPGNLDAAVYSPRTRRTHFFKGNKVWRYVDFKMSPGFPMKFNRVEPNLDAALYWPVNQKVFLFKGSGYWQWDELARTDLSRYPKPIKELFTGVPDRPSAAMSWQDGQVYFFKGKEYWRLNQQLRVAKGYPRNTTHWMHCGSQ

SEQ ID NO: 49 - protein, 191 aa, Bos taurus
PDLCDSNLSFDAVTMLGKELLLFRDRIFWRRQVHLMSGIRPSTITSSFPQLMSNVDAAYEVAERGTAYFFKGPHYWITRGFQMQGPPRTIYDFGFPRYVQRIDAAVYLKDAQKTLFFVGDEYYSYDERKRKMEKDYPKSTEEEFSGVNGQIDAAVELNGYIYFFSGPKAYKSDTEKEDVVSELKSSSWIGC

SEQ ID NO: 50 - protein, 191 aa, Homo sapiens
PDLCDSSSSFDAVTMLGKELLLFKDRIFWRRQVHLRTGIRPSTITSSFPQLMSNVDAAYEVAERGTAYFFKGPHYWITRGFQMQGPPRTIYDFGFPRHVQQIDAAVYLREPQKTLFFVGDEYYSYDERKRKMEKDYPKNTEEEFSGVNGQIDAAVELNGYIYFFSGPKTYKYDTEKEDVVSVVKSSSWIGC

SEQ ID NO: 51 - protein, 191 aa, Mus musculus
PDLCDSSSSFDAVTMLGKELLFFKDRIFWRRQVHLPTGIRPSTITSSFPQLMSNVDAAYEVAERGIAFFFKGPHYWVTRGFHMQGPPRTIYDFGFPRHVQRIDAAVYLKEPQKTLFFVGEEYYSYDERKKKMEKDYPKNTEEEFSGVSGHIDAAVELNGYIYFFSGRKTFKYDTEKEDVVSVVKSSSWIGC

SEQ ID NO: 52 - protein, 191 aa, Sus scrofa
PDICDSSSSFDAVTMLGKELLFFRDRIFWRRQVHLMSGIRPSTITSSFPQLMSNVDAAYEVADRGMAYFFKGPHYWITRGFQMQGPPRTIYDFGFPRYVQRIDAAVHLKDTQKTLFFVGDEYYSYDERKRKMDKDYPKNTEEEFSGVNGQIDAAVELNGYIYFFSGPKAYKYDTEKEDVVSVLKSNSWIGC

SEQ ID NO: 53 - protein, 196 aa, Homo sapiens
PNICDGNFNTVALFRGEMFVFKDRWFWRLRNNRVQEGYPMQIEQFWKGLPARIDAAYERADGRFVFFKGDKYWVFKEVTVEPGYPHSLGELGSCLPREGIDTALRWEPVGKTYFFKGERYWRYSEERRATDPGYPKPITVWKGIPQAPQGAFISKEGYYTYFYKGRDYWKFDNQKLSVEPGYPRNILRDWMGCNQK

SEQ ID NO: 54 - protein, 196 aa, Mus musculus
PNICDGNFNTVALFRGEMFVFKDRWFWRLRNNRVQEGYPMQIEQFWKGLPARIDAAYERADGRFVFFKGDKYWVFKEVTVEPGYPHSLGELGSCLPREGIDTALRWEPVGKTYFFKGERYWRYSEERRATDPGYPKPITVWKGIPQAPQGAFISKEGYYTYFYKGRDYWKFDNQKLSVEPGYPRNILRDWMGCKQK

SEQ ID NO: 55 - protein, 196 aa, Rattus norvegicus; identical to SEQ ID NO: 54

SEQ ID NO: 56 - protein, 198 aa, Homo sapiens
PDRCEGNFDAIANIRGETFFFKGPWFWRLQPSGQLVSPRPARLHRFWEGLPAQVRVVQAAYARHRDGRILLFSGPQFWVFQDRQLEGGARPLTELGLPPGEEVDAVFSWPQNGKTYLVRGRQYWRYDEAAARPDPGYPRDLSLWEGAPPSPDDVTVSNAGDTYFFKGAHYWRFPKNSIKTEPDAPQPMGPNWLDCPAP

SEQ ID NO: 57 - protein, 193 aa, Homo sapiens
FDAITVDRQQQLYIFKGSHFWEVAADGNVSEPRPLQERWVGLPPNIEAAAVSLNDGDFYFFKGGRCWRFRGPKPVWGLPQLCRAGGLPRHPDAALFFPPLRRLILFKGARYYVLARGGLQVEPYYPRSLQDWGGIPEEVSGALPRPDGSIIFFRDDRYWRLDQAKLQATTSGRWATELPWMGCWHANSGSALF

SEQ ID NO: 58 - protein, 191 aa, Homo sapiens
PSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSRATWLSLFSSEESNLGANNYDDYRMDWLVPATCEPIQSVFFFSGDKYYRVNLRTRRVDTVDPPYPRSIAQYWLGCPAPGHL

SEQ ID NO: 59 - protein, 192 aa, Mus musculus
PSQEECEGSSLSAVFEHFALLQRDSWENIFELLFWGRSSDGAREPQFISRNWHGVPGKVDAAMAGRIYVTGSLSHSAQAKKQKSKRRSRKRYRSRRGRGHRRSQSSNSRRSSRSIWFSLFSSEESGLGTYNNYDYDMDWLVPATCEPIQSVYFFSGDKYYRVNLRTRRVDSVNPPYPRSIAQYWLGCPTSEK

SEQ ID NO: 60 - protein, 195 aa, Sus scrofa
PSREECEGSSPSDVFAHFALMQRDSWEDIFRLLFWSHSFGGAIEPRVISQDWLGLPEQVDAAMAGQIYISGSALKPSQPKMTKSARRSGKRYRSRRGRGRGRGHSRSQKSHRQSRSTWLPWFSSEETGPGGYNYDDYKMDWLVPATCEPIQSVYFFSGEEYYRVNLRTQRVDTVTPPYPRSIAQYWLGCPVPDQK

SEQ ID NO: 61 - protein, 188 aa, Oryctolagus cuniculus
PSQEECEGSSLSAVFEHFAMLHRDSWEDIFKLLFWGRPSGGARQPQFISRDWHGVPGKVDAAMAGRIYISGLTPSPSAKKQKSRRRSRKRYRSRYGRGRSQNSRRLSRSISRLWFSSEEVSLGPYNYEDYETSWLKPATSEPIQSVYFFSGDKYYRVNLRTQRVDTVNPPYPRSIAQYWLGCPAPGGQ

SEQ ID NO: 62 - DNA, 345 bp, Artificial Sequence
  Description of Artificial Sequence Synthetic polynucleotide
gctggctctggagctggtgcaggctctggtgctggcgcaggttctggcgctggtgctggttctggcactggtgcttctccggcagctgttccggcagcggttccagcagcggtgccggcagcagttcctgctgcggtgggcgaaggagaaggagaaggcgagggagagggcgaaggatacccgtacgacgtaccggactacgccgaaggtggtggtggctccgagcagaagctcatctccgaagaagacctggagggtggtggtggctccacagactacaaggacgacgacgacaaatcccaaatcaaacgaaaggcccagtcgaaaaactgggcctttcgattt

SEQ ID NO: 63 - DNA, 22 bp, Artificial Sequence
  Description of Artificial Sequence Synthetic oligonucleotide
atgcctgaaatctgcaaacagg

SEQ ID NO: 64 - DNA, 54 bp, Artificial Sequence
  Description of Artificial Sequence Synthetic oligonucleotide
cctgcaaagaacacagctttctcmnnmnntggggcctcgtataccgcatcaatc

SEQ ID NO: 65 - DNA, 42 bp, Artificial Sequence
  Description of Artificial Sequence Synthetic oligonucleotide
ggaccctggcttccccaagctcatcgcagatgcctggaatgc

SEQ ID NO: 66 - DNA, 19 bp, Artificial Sequence
  Description of Artificial Sequence Synthetic oligonucleotide
ggagctcgctcgagtcagc

SEQ ID NO: 67 - DNA, 60 bp, Artificial Sequence
  Description of Artificial Sequence Synthetic oligonucleotide
gagaaagctgtgttctttgcagggaatgaatactggatctactcagcgagcaccttggag

SEQ ID NO: 68 - DNA, 60 bp, Artificial Sequence
  Description of Artificial Sequence Synthetic oligonucleotide
gagcttggggaagccagggtccattttmnncttmnnctcgttgtacctccagaacttgtc

SEQ ID NO: 69 - DNA, 57 bp, Artificial Sequence
  Description of Artificial Sequence Synthetic oligonucleotide
ggcagtccgaggctagtcagtggtttgggataaccmnnctccaaggtgctcgctgag

SEQ ID NO: 70 - DNA, 52 bp, Artificial Sequence
  Description of Artificial Sequence Synthetic oligonucleotide
gccagcgaagatgtatgtcttmnnmnnmnngctccagttaaaggctgcatcc

SEQ ID NO: 71 - DNA, 60 bp, Artificial Sequence
  Description of Artificial Sequence Synthetic oligonucleotide
ctgactagcctcggactgccccctgatgttcaacgtgtggatgcagcctttaactggagc

SEQ ID NO: 72 - DNA, 55 bp, Artificial Sequence
  Description of Artificial Sequence Synthetic oligonucleotide
gtttaactttaagaaggagatatacatatgcctgaaatctgcaaacaggatatcg

SEQ ID NO: 73 - DNA, 49 bp, Artificial Sequence
  Description of Artificial Sequence Synthetic oligonucleotide
cctgcaccagctccagagccagcgcagcctagccagtcggatttgatgc

SEQ ID NO: 74 - DNA, 186 bp, Artificial Sequence
  Description of Artificial Sequence Synthetic polynucleotide
ggtgatgtcggcgatataggcgccagcaaccgcacctgtggcgccggtgatgccggccacgatgcgtccggcgtagaggatcgagatctcgatcccgcgaaattaatacgactcactatagggagaccacaacggtttccctctagaaataattttgtttaactttaagaaggagatatacatatg

SEQ ID NO: 75 - DNA, 27 bp, Artificial Sequence
  Description of Artificial Sequence Synthetic oligonucleotide
ggtgatgtcggcgatataggcgccagc

SEQ ID NO: 76 - DNA, 23 bp, Artificial Sequence
  Description of Artificial Sequence Synthetic oligonucleotide
aaatcgaaaggcccagtttttcg

SEQ ID NO: 77 - DNA, 55 bp, Artificial Sequence; identical to SEQ ID NO: 72
  Description of Artificial Sequence Synthetic primer

SEQ ID NO: 78 - DNA, 50 bp, Artificial Sequence
  Description of Artificial Sequence Synthetic primer
gtcgttcagaccacgaccctcgatgcagcctagccagtcggatttgatgc

SEQ ID NO: 79 - DNA, 243 bp, Artificial Sequence
  Description of Artificial Sequence Synthetic polynucleotide
ggtgatgtcggcgatataggcgccagcaaccgcacctgtggcgccggtgatgccggccacgatgcgtccggcgtagaggatcgagatctcgatcccgcgaaattaatacgactcactatagggagaccacaacggtttccctctagaaataattttgtttaactttaagaaggagatatacatatgggtcttaatgatatttttgaagctcagaaaatcgaatggcacgaaatcgagggtcgt

SEQ ID NO: 80 - DNA, 332 bp, Artificial Sequence
  Description of Artificial Sequence Synthetic polynucleotide
taggaagctcagaaaatcgaatggcacgaataatgagctcccgggagcgcttggagccacccgcagttcgaaaaataataagggcctcccactgactgctcttctgtcagtgggctactcctggactcggcaccagattgcctcatttttctcctctggcattttgtataaatccaccttgactggggaaattctcctggggtcaggtggcaccagcctggatccggctgctaacaaagcccgaaaggaagctgagttggctgctgccaccgctgagcaataactagcataaccccttggggcctctaaacgggtcttgaggggttttttgc

SEQ ID NO: 81 - DNA, 30 bp, Artificial Sequence
  Description of Artificial Sequence Synthetic oligonucleotide
gcaaaaaacccctcaagacccgtttagagg

SEQ ID NO: 82 - DNA, 22 bp, Artificial Sequence
  Description of Artificial Sequence Synthetic oligonucleotide
ggtgatgtcggcgatataggcg

SEQ ID NO: 83 - DNA, 37 bp, Artificial Sequence
  Description of Artificial Sequence Synthetic oligonucleotide
ctgagagtgcaccatatgcctgaaatctgcaaacagg

SEQ ID NO: 84 - DNA, 33 bp, Artificial Sequence
  Description of Artificial Sequence Synthetic oligonucleotide
ccatgattacgaattcgcagcctagccagtcgg

SEQ ID NO: 85 - DNA, 38 bp, Artificial Sequence
  Description of Artificial Sequence Synthetic oligonucleotide
ttaagctgctaaagcgtagttttcgtcgtttgcgacta

SEQ ID NO: 86 - DNA, 28 bp, Artificial Sequence
  Description of Artificial Sequence Synthetic primer
cagagcctgcaccagctccagagccagc

SEQ ID NO: 87 - DNA, 30 bp, Artificial Sequence
  Description of Artificial Sequence Synthetic primer
gtttaactttaagaaggagatatacatatg

SEQ ID NO: 88 - protein, 210 aa, Artificial Sequence
  Description of Artificial Sequence Synthetic polypeptide
PEICKQDIVFDGIAQIRGEXIFFFKDRFIWRTVXTPRDKPMGPLLVATFWPELPXXXEKIDAVYEAPQEEKAVFFAGNEYWIYSASTLERGYPKPLXTSLGLPPDVQRVDAAFNWSKNXXKKTYIFAGDKFWRYNEVKKKMDPGFPKLIADAWNAIPDNLDAVVDLQGGGHSYFFKGAYYLKLENQSLXXXXXXXKSVKFGSIKSDWLGC

(19) United States
(12) Patent Application Publication
(10) Pub. No.: US 2009/0054252 A1
(43) Pub. Date: Feb. 26, 2009

(54) Hemopexin-Like Structure as New Polypeptide-Scaffold

(76) Inventors: Martin Lanzendoerfer, Tutzing (DE); Michael Schraeml, Halle (DE)

Correspondence Address: George W. Johnston;Hoffmann-La Roche, 340 Kingsland Street, Nutley, NJ, 07110, US

(21) Appl. No.: 11/794,415

(22) PCT Filed: Jan. 2, 2006
(86) PCT No.: PCT/EP2006/000004

§ 371 Date: Jun. 26, 2007

(30) Foreign Application Priority Data
Jan. 3, 2005 (EP) 05000013.2

(51) Int. Cl. C40B 30/04; C07K 14/00; G01N 33/00; C12N 15/00; C12P 21/04

(52) U.S. Cl. 506/9; 530/350; 435/69.1; 436/86; 435/320.1

(57) ABSTRACT

The invention concerns a method for the generation of a polypeptide with specific binding properties to a predetermined target molecule which are not naturally inherent to that polypeptide. At the same time an optimization of the binding specifity and a process of production are described. The invention further concerns a method for the identification and modification of specific amino acid positions within a polypeptide scaffold.

The invention concerns a method for the preparation of a polypeptide with specific binding properties to a predetermined target molecule which are not naturally inherent to that polypeptide. At the same time a process for the optimization of the binding specifity and a process of production are described. The invention further concerns a method for the identification and modification of alterable amino acid positions within a polypeptide scaffold.

### TECHNOLOGICAL BACKGROUND

In recent years the number of applications and publications related to affinity reagents steadily increased. The majority thereof is related to antibodies, i.e. monoclonal or polyclonal immunoglobulines. Only a minor part deals with possible alternatives. One of these is the use of protein scaffolds. This concept requires a stable protein architecture tolerating multiple substitutions or insertions at the primary structural level (Nygren, P-A., Skerra, A., J. Immun. Meth. 290 (2004) 3-28).

Modified protein scaffolds can overcome existing problems and extent the application area of affinity reagents. Only one problem among many others is the intracellular application of antibodies. The bottleneck of this protein knockout technology is that not all antibodies expressed within cells perform well. This is called the “disulphide bond problem”. To overcome this problem time-consuming experiments have to be performed to optimize a complex list of parameters (Visintin, M., et al., J. Immun. Meth. 290 (2004) 135-153).

In this regard proteins possess several advantages. Among these are low molecular weight, ease of production by microorganisms, simplicity to modify and broad applicability. Different protein scaffolds have been described in this context, e.g. Zinc-finger proteins for DNA recognition (Segal, D. J., et al., Biochem. 42 (2003) 2137-2148); Thioredoxin based peptide aptamers modified by introduction of variable polypeptide sequences in the active-site loop (Klevenz, B., et al., Cell. Mol. Life. Sci. 59 (2002) 1993-1998); Protein A as “Affibody” scaffold (Sandstrom, K., et al., Prot. Eng. 16 (2003) 691-697; Andersson, M., et al., J. Immun. Meth. 283 (2003) 225-234); mRNA-protein molecules of the tenth fibronectin type III domain (Xu, L., et al., Chem. Biol. 9 (2002) 933-942) or alpha-Amylase inhibitor based binding molecules (McConnell, S. J., and Hoess, R. H., J. Mol. Biol. 250 (1995) 460-470).

Two criteria substantially characterize an applicable protein-scaffold: First, the protein should belong to a family which reveals a well defined hydrophobic core. A close relationship between the individual family members is beneficial (Skerra, A., J. Mol. Recognit. 13 (2000) 167-187). Second, the protein should possess a spatially separated and functionally independent accessible active site or binding pocket. This should not contribute to the intrinsic core-stability (Predki, P. F., et al., Nature Struct. Biol. 3 (1996) 54-58). Ideally, this protein-family is inherently involved in the recognition of multiple, non-related targets.

As described in Nygren, P-A., and Skerra, A., J. Immun. Meth. 290 (2004) 3-28 several polypeptide scaffolds have been employed for the development of novel affinity proteins. These scaffolds can be divided into three groups: (i) single peptide loops, (ii) engineered interfaces and (iii) non-contiguous hyper variable loops.

With the scaffolds of the first group either single amino acids in an exposed loop are diversified or small polypeptide sequences are inserted into this exposed loop (see e.g. Roberts, B. L., et al., PNAS 89 (1992) 2429-2433 and Gene 121 (1992) 9-16; Röttgen, P., and Collins, J., Gene 164 (1995) 243; Lu, Z., et al., Bio/Technology 13 (1995) 366-372). One drawback of this approach is, that affinity, if any, to a completely novel target is difficult to achieve (Klevenz, B., et al., Cell. Mol. Life. Sci. 59 (2002) 1993-1998). The intrinsic binding affinity to the natural or closely related targets can be modified, but the target or the target class can hardly be changed. Another drawback is that in the case of insertion of small randomized polypeptides, the target has to be known and these sequences have to be generated beforehand based on already established knowledge.

To the scaffolds of the second group belong e.g. the immunoglobulin binding domain of Staphylococcal protein A (e.g. Sandstrom, K., et al., Prot. Eng. 16 (2003) 691-697), the C-terminal cellulose-binding domain of cellobiohydrolase I of the fungus T. reesei (Smith, G. P., et al., J. Mol. Biol. 277 (1998) 317-322) and the gamma-crystallines (Fiedler, U., and Rudolph, R., WO 01/04144).

The third class is represented by the immunoglobulin itself and the distantly related fibronectin type III domain as well as some classes of neurotoxins.

Beside the suitability as scaffold for the generation of specific binding characteristics to predetermined target molecules, the application conditions have to be considered. Among other things especially the stability, selectivity, solubility and functional production of the affinity polypeptide have to be taken into account. As an example, already mentioned above, the bottleneck of the protein knockout technology is that not all antibodies expressed as affinity molecules within cells are functionally produced (“disulphide bond problem”, Visintin, M., et al., J. Immun. Meth. 290 (2004) 135-153).

Therefore it is the objective of the current invention to overcome these drawbacks by providing an alternative polypeptide scaffold with specific binding properties to a predetermined target molecule which are not naturally inherent to that polypeptide. This comprises the randomization of amino acids, the optimization of the binding characteristics and a method of production of the optimized polypeptide with specific binding properties.

### SUMMARY OF THE INVENTION

The present invention provides a polypeptide, that specifically binds a predetermined target molecule, characterized in that the amino acid sequence of the polypeptide is selected from the group consisting of SEQ ID NO:02 to SEQ ID NO:61, wherein in said amino acid sequence at least one amino acid according to table V is altered.

The invention further comprises a process for the production of a polypeptide specifically binding a predetermined target molecule in a prokaryotic or eukaryotic microorganism, characterized in that said microorganism contains a gene which encodes said polypeptide and said polypeptide is expressed.

The invention further comprises a vector for the expression of the polypeptide that specifically binds a predetermined target molecule in a prokaryotic or eukaryotic microorganism.

The polypeptide can be isolated and purified by methods known to a person skilled in the art.

In another embodiment of the invention the predetermined target molecule is a member of one of the groups consisting of hedgehog proteins, bone morphogenetic proteins, growth factors, erythropoietin, thrombopoietin, G-CSF, interleukins and interferons.

The invention further provides a method for identifying a nucleic acid encoding a polypeptide which specifically binds a target molecule from a DNA-library, wherein the method comprises the steps of
a) selecting a sequence from the group consisting of SEQ ID NO:02 to SEQ ID NO:61;
b) preparing a DNA-library of the selected sequence in which at least one amino acid position according to table V is altered;
c) screening the prepared DNA-library for encoded polypeptides specifically binding a predetermined target molecule;
d) choosing the nucleic acid encoding one specific binder identified in step c);
e) repeating the steps b) to d) for two to five times; and
f) isolating said nucleic acid encoding a polypeptide specifically binding a predetermined target molecule.

In another embodiment the method for identifying a nucleic acid encoding a polypeptide which specifically binds a target molecule from a DNA-library, comprises linear expression elements.

In another embodiment the library of the polypeptide is expressed by display on ribosomes.

In another embodiment the library of the polypeptide is expressed by display on bacteriophages.

The invention further comprises a method for the determination of alterable amino acid positions in a polypeptide comprising the steps of
a) assembling of a plurality of sequences of polypeptides which are homologous in structure and/or function from the same and/or different organisms; and
b) aligning the sequences according to a common structural and/or consensus sequence and/or functional motif; and
c) determining the variability of all amino acids positions in the alignment by counting the number of different amino acids found for each position of the sequence; and
d) identifying alterable amino acid positions as amino acid positions with a total number of different amino acids of eight or more.

### DETAILED DESCRIPTION OF THE INVENTION

The present invention provides a polypeptide, that specifically binds a predetermined target molecule, characterized in that the amino acid sequence of the polypeptide is selected from the group consisting of SEQ ID NO:02 to SEQ ID NO:61, wherein in said amino acid sequence at least one amino acid according to table V is altered. The invention further provides a method for identifying a nucleic acid encoding a polypeptide which specifically binds a predetermined target molecule from a DNA-library and a method for the determination of alterable amino acid positions in a polypeptide.

The polypeptide can be defined by its amino acid sequence and by the DNA sequence derived there from.

The polypeptide according to the invention can be produced by recombinant means, or synthetically.

The use of recombinant DNA technology enables the production of numerous derivatives of the polypeptide. Such derivatives can, for example, be modified in individual or several amino acid positions by substitution, alteration or exchange. The derivatisation can, for example, be carried out by means of site directed mutagenesis. Such variations can easily be carried out by a person skilled in the art (Sambrook, J., et al., Molecular Cloning: A laboratory manual (1999) Cold Spring Harbor Laboratory Press, New York, USA; Hames, B. D., and Higgins, S. G., Nucleic acid hybridization—a practical approach (1985) IRL Press, Oxford, England).

The invention further comprises a process for the production of a polypeptide specifically binding a predetermined target molecule in a prokaryotic or eukaryotic microorganism, characterized in that said microorganism contains a nucleic acid sequence which encodes said polypeptide and said polypeptide is expressed. The invention therefore in addition concerns a polypeptide which is a product of prokaryotic or eukaryotic expression of an exogenous nucleic acid molecule according to the invention. With the aid of such nucleic acids, the polypeptide according to the invention can be obtained in a reproducible manner in large amounts. For expression in eukaryotic or prokaryotic host cells, the nucleic acid, encoding the amino acid sequence of the polypeptide, is integrated into suitable expression vectors, according to methods familiar to a person skilled in the art. Such an expression vector preferably contains a regulable or inducible promoter. These recombinant vectors are then introduced for expression into suitable host cells such as, e.g., E. coli as a prokaryotic host cell or Saccharomyces cerevisiae, insect cells or CHO cells as eukaryotic host cells and the transformed or transduced host cells are cultured under conditions which allow expression of the heterologous gene.

The polypeptide can be isolated and purified after recombinant production by methods known to a person skilled in the art, e.g. by affinity chromatography using known protein purification techniques, including immunoprecipitation, gel filtration, ion exchange chromatography, chromatofocussing, isoelectric focusing, selective precipitation, electrophoresis, or the like (see e.g. Ausubel, I., and Frederick, M., Curr. Prot. Mol. Biol. (1992) John Wiley and Sons, New York; Sambrook, J., et al., Molecular Cloning: A laboratory manual (1999) Cold Spring Harbor Laboratory Press, New York, USA; Hames, B. D., and Higgins, S. G., Nucleic acid hybridization—a practical approach (1985) IRL Press, Oxford, England).

The following abbreviations and definitions are used within this invention.

A “polypeptide” is a polymer of amino acid residues joined by peptide bonds, whether produced naturally or synthetically. Polypeptides of less than about 20 amino acid residues may be referred to as “peptides”; polypeptides of more than about 100 amino acid residues may be referred to as “proteins”.

The term “hemopexin-like domain” (PEX) stands for a polypeptide which displays sequence and structure homology to the blood protein hemopexin. This domain has a mean sequence of about 200 amino acids and consists of four repeating sub domains.

The abbreviation “PEX2” stands for the C-terminal domain of human matrix-metalloproteinase 2, comprising the amino acid positions 466 to 660 of the full length protein.

The term “consensus sequence” stands for a deduced sequence, either nucleotide or amino acid sequence. This sequence represents a plurality of similar sequences. Each position in the consensus sequence corresponds to the most frequently occurring base or amino acid at that position which is determined by aligning three or more sequences.

The term “alter” stands for a process in which a defined position in a sequence, either nucleic acid sequence or amino acid sequence, is modified. This comprises the replacement of an amino acid or a nucleic acid (nucleotide) with a different amino acid or nucleic acid (nucleotide) as well as the deletion or insertion.

The expression “a polypeptide binding a molecule” stands for a polypeptide that has the ability to bind a target molecule. The term “specifically binds” stands for a binding activity with an affinity constant of more than 10 E 7 (10⁷) liters/mole.

The expression “predetermined target molecule” denotes a molecule which is a member of the groups of proteins comprising hedgehog proteins, bone morphogenetic proteins, growth factors, erythropoietin, thrombopoietin, G-CSF, interleukins and interferons, immunoglobulins, enzymes, inhibitors, activators, and cell surface proteins.

The term “expression vector” or “vector” stands for a natural or artificial DNA sequence comprising at least a nucleic acid sequence encoding the amino acid sequence of a polypeptide, a promoter sequence, a terminator sequence, a selection marker and an origin of replication.

The term “nucleic acid molecule” or “nucleic acid” stands for a polynucleotide molecule which can be, e.g., DNA, RNA or derivatives thereof. Due to the degeneracy of the genetic code, different nucleic acid sequences encode the same polypeptide. These variations are also included.

The term “amino acid” stands for alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

The term “amino acid diversity number” stands for the number of different amino acids present at a specific position of an amino acid sequence. This number is determined by aligning the sequences of an assembly of a plurality of sequences of polypeptides which are homologous in structure and/or function from the same and/or different organisms to a reference or consensus sequence and identifying the total number of different amino acids present in all aligned sequences at the specific position.

The term “aligning” stands for the process of lining up two or more sequences to achieve maximal levels of identity and conservation. It comprises the determination of positional homology for molecular sequences, involving the juxtaposition of amino acids or nucleotides in homologous molecules. As a result the compared sequences are presented in a form that the regions of greatest statistical similarity are shown. During this process it may be found that some sequences do not contain all positions of other aligned sequences, i.e. it may be possible that sequences contain one or more deletions. To achieve maximal levels of identity and conservation gaps can be introduced in these sequences. The gaps are denoted by hyphens in the illustration of the alignment.

The terms “Overlapping Extension Ligation PCR” (OEL-PCR) and “Linear Expression Element” (LEE) stand for a method to ligate DNA fragments and describe linear DNA fragments used in and obtained by this method (see e.g. Ho, S. N., et al., Gene 77 (1989) 51-59; Kain, K. C., et al., Biotechniques 10 (1991) 366-374; Shuldiner, A. R., et al., Anal. Biochem. 194 (1991) 9-15). A gene-transcript is segmented into the modules “promotor-module”, “gene-module” and “terminator-module”. The promotor-module encodes the T7 phage transcription promotor sequence, a translation control sequence (RBS=ribosomal binding site) and the T7 phage enhancer sequence (gloepsilon) (Lee, S. S., and Kang, C., Kor. Biochem. J. 24 (1991) 673-679). These regulatory sequences enable a coupled transcription and translation in a rapid translation system, e.g. RTS 100 E. coli HY System from Roche Applied Sciences, Mannheim, Germany. The terminator-module encodes a translation stop-codon and a palindromic T7 phage termination-motif (T7T). Optionally, these modules comprised DNA sequences encoding polypeptides, which can be used in subsequent affinity purification or labeling procedures. Linear Expression Elements (Sykes, K. F., and Johnston, S. A., Nature Biotechnol. 17 (1999) 355-359) were assembled by these modules by a two-step PCR. In a first standard PCR using the Pyrococcus woesii DNA-polymerase (PWO-PCR) an intron-less open reading frame, i.e. the gene-module, is amplified by sequence-specific flanking primer oligonucleotides, which introduce overlapping complementary sequences to the promotor- and terminator-modules. The PCR-mediated ligation of these DNA fragments requires a free hybridization energy of the complementary sequences, which has to be lower than a delta G of −25 kcal/mol. This is achieved by sequence extensions, which are in average 25 bp in length. The primer oligonucleotides are designed to hybridize with the gene template at a temperature between 48° C. to 55° C. This enforces the use of primer oligonucleotides with an average length of 45 bp to 55 bp. After 30 PCR cycles the PCR mixture containing approximately 50 ng of the elongated gene-module DNA is transferred into a second PCR mixture. This PCR is supplied with 50 ng to 100 ng of the respective promotor- and terminator-DNA-modules and sequence specific terminal primers. In the presence of a DNA-polymerase the 3′-ends of the hybridized complementary DNA-fragments are enzymatic elongated (Barik, S., Meth. Mol. Biol. 192 (2002) 185-196) to a full length DNA transcript comprising all three modules.

The term “microorganism” denotes prokaryotic microorganisms and eukaryotic microorganisms. The “microorganism” is preferably selected from the group consisting of E. coli strains, Bacillus subtilis strains, Klebsiella strains, Salmonella strains, Pseudomonas strains or Streptomyces strains and yeast strains. For example, E. coli strains comprises E. coli-K12, UT5600, HB101, XL1, X1776, W3110; yeast strains, e.g., comprises Saccharomyces, Pichia, Hansenula, Kluyveromyces and Schizosaccharomyces.

Two criteria substantially characterize an applicable protein-scaffold: First, the protein should belong to a family which reveals a well defined hydrophobic core. A close relationship between the individual family members is beneficial (Skerra, A., J. Mol. Recognit. 13 (2000) 167-187). Second, the protein should posses a spatially separated and functionally independent accessible active site or binding pocket. This should not contribute to the intrinsic core-stability (Predki, P. F., et al., Nature Struct. Biol. 3 (1996) 54-58). Ideally, this protein-family is inherently involved in the recognition of multiple, non-related targets.

The hemopexin-like (PEX) protein scaffold fulfills these criteria. This structural motive is present in a plurality of different proteins and protein families, e.g. hemopexin (Altruda, F. et al., Nucleic Acids Res. 13 (1985) 3841-3859), vitronectin (Jenne, D., and Stanley, K. K., Biochemistry 26 (1987) 6735-6742) or pea seed albumin 2 (Jenne, D., Biochem. Biophys. Res. Commun. 176 (1991) 1000-1006).

The crystal structure analyses of proteins containing hemopexin-like domains show that this domain adopts a four bladed beta-propeller topology (Li, J., et al., Structure 3 (1995) 541-549; Faber, H. R., et al., Structure 3 (1995) 551-559). The blades are each composed of four beta-sheets in an anti-parallel orientation. Together they form a cavity in the center of the molecule. The four blades are linked together via loops from the fourth outermost beta-strand of the preceding blade to the first innermost beta-strand of the next blade. A disulphide bond connects the terminal ends of the structure, i.e. blade 4 and blade 1.

The PEX scaffold is involved in different, but quite specific protein-protein- and protein-ligand-interactions. Therefore the hemopexin-like structure forms a versatile framework for molecular recognition (Bode, W., Structure 3 (1995) 527-530). For example, binding sites for ions like calcium, sodium and chloride (Libson, A. M., et al., Nat. Struct. Biol. 2 (1995) 938-942; Gohlke, U., et al., FEBS Lett. 378 (1996) 126-130) as well as for interaction with fibronectin, TIMP-1/2 (tissue inactivator of human matrix metalloproteinase 1/2), integrins and heparin are known (Wallon, U. M., and Overall, C. M., J. Biol. Chem. 272 (1997) 7473-7481; Willenbrock, F., et al., Biochemistry 32 (1993) 4330-4337; Brooks, P. C., et al., Cell 92 (1998) 391-400; Bode, W., Structure 3 (1995) 527-530).

The hemopexin-like protein domain offers a high structural homology among its protein family members. High structural equivalence of the hemopexin-domains of e.g. human Matrix-metalloproteinases 1, 2 and 13 has been reported (Gomis-Ruth, F. X., et al., J. Mol. Biol. 264 (1996) 556-566). The predominantly hydrophobic interactions between the adjacent and perpendicularly oriented beta-sheets provide most of the required structural stability (Gomis-Ruth, F. X., et al., J. Mol. Biol. 264 (1996) 556-566; Fulop, V., and Jones, D. T., Curr. Opin. Struct. Biol. 9 (1999) 715-721).

Protein-databases like SMART (Schultz, J., et al., PNAS 95 (1998) 5857-5864; Letunic, I., et al., Nuc. Acids Res. 30 (2002) 242-244) were recently used to compare homologous sequences and protein-folds in order to identify non-conserved, and thus theoretically alterable, i.e. randomizable, amino acid positions in suitable protein frameworks (Binz, H. K., et al., J. Mol. Biol. 332 (2003) 489-503; Forrer, P., et al., ChemBioChem 5 (2004) 183-189).

To identify potentially alterable, i.e. randomizable, amino acid positions in the PEX fold, a similar approach was performed using the SMART database. Amino acid positions were identified in the PEX domain, which are randomizable without affecting the proteins structure, functional conformation and stability.

From the SMART database 60 PEX-domains, that are listed in the following table I, from different species were aligned with the Pretty bioinformatics tool (GCG) using the scoring matrix blosum 62.

TABLE I
Listing of the 60 proteins containing the PEX-domain.

| | | | sequence id of the |
|---|---|---|---|
| | | | hemopexin |
| | | | domain as used in |
| | PEX-fold in PDB data | swissprot data bank | this invention |
| Protein family | bank code | number | (SEQ ID NO:) |
| peroxisome | PEX2_mouse | P55098 | 03 |
| assembly factor | PEX2_rat | P24392 | 04 |
| matrix | MM01_Bovin | P28053 | 06 |
| metalloproteinase 1 | MM01_HORSE | Q9XSZ5 | 07 |
| | MM01_human | P03956 | 08 |
| | MM01_PIG | P21692 | 09 |
| matrix | MM02_chick (chicken) | Q90611 | 02 |
| metalloproteinase 2 | MM02_human | P08253 | 10 |
| | MM02_rabbit | P50757 | 05 |
| matrix | MM03_human | P08254 | 11 |
| metalloproteinase 3 | MM03_MOUSE | P28862 | 12 |
| | MM03_RABIT | P28863 | 13 |
| | MM03_RAT | P03957 | 14 |
| matrix | MM08_human | P22894 | 15 |
| metalloproteinase 8 | MM08_MOUSE | O70138 | 16 |
| | MM08_RAT | O88766 | 17 |
| matrix | MM09_BOVIN | P52176 | 18 |
| metalloproteinase 9 | MM09_CANFA | O18733 | 19 |
| | (canis familiaris, dog) | | |
| | MM09_human | P14780 | 20 |
| | MM09_MOUSE | P41245 | 21 |
| matrix | MM10_human | P09238 | 22 |
| metalloproteinase | MM10_MOUSE | O55123 | 23 |
| 10 | | | |
| matrix | MM11_human | P24347 | 24 |
| metalloproteinase | MM11_MOUSE | Q02853 | 25 |
| 11 | | | |
| matrix | MM12_human | P39900 | 26 |
| metalloproteinase | MM12_MOUSE | P34960 | 27 |
| 12 | MM12_RABIT | P79227 | 28 |
| | MM12_RAT | Q63341 | 29 |
| matrix | MM13_BOVIN | O77656 | 30 |
| metalloproteinase | MM13_HORSE | O18927 | 31 |
| 13 | MM13_human | P45452 | 32 |
| | MM13_RABIT | O62806 | 33 |
| matrix | MM14_human | P50281 | 34 |
| metalloproteinase | MM14_mouse | P53690 | 35 |
| 14 | MM14_PIG | Q9XT90 | 36 |
| | MM14_RABIT | Q95220 | 37 |
| | MM14_RAT | Q10739 | 38 |
| matrix | MM15_human | P51511 | 39 |
| metalloproteinase | MM15_MOUSE | O54732 | 40 |
| 15 | | | |
| matrix | MM16_human | P51512 | 41 |
| metalloproteinase | MM16_MOUSE | Q9WTR0 | 42 |
| 16 | MM16_RAT | O35548 | 43 |
| matrix | MM17_human | Q9ULZ9 | 44 |
| metalloproteinase | MM17_MOUSE | Q9R0S3 | 45 |
| 17 | | | |
| matrix | MM18_XENLA | O13065 | 46 |
| metalloproteinase | (Xenopus laevis, African | | |
| 18 | clawed frog) | | |
| matrix | MM19_human | Q99542 | 47 |
| metalloproteinase | MM19_MOUSE | Q9JHI0 | 48 |
| 19 | | | |
| matrix | MM20_BOVIN | O18767 | 49 |
| metalloproteinase | MM20_human | O60882 | 50 |
| 20 | MM20_MOUSE | P57748 | 51 |
| | MM20_PIG | P79287 | 52 |
| matrix | MM24_human | Q9Y5R2 | 53 |
| metalloproteinase | MM24_MOUSE | Q9R0S2 | 54 |
| 24 | MM24_RAT | Q99PW6 | 55 |
| matrix | MM25_human | Q9NPA2 | 56 |
| metalloproteinase | | | |
| 25 | | | |
| matrix | MM28_human | Q9H239 | 57 |
| metalloproteinase | | | |
| 28 | | | |
| vitronectin | VTNC_human | P04004 | 58 |
| | VTNC_MOUSE | P29788 | 59 |
| | VTNC_PIG | P48819 | 60 |
| | VTNC_RABIT | P22458 | 61 |

(Table I end).

The hemopexin-like domain just accounts for a small part of the full length protein.

The following table lists the location of the aligned hemopexin-like domain in the full length proteins.

TABLE II
Location of the hemopexin-like domains in the proteins of table I.

| | |
|---|---|
| | hemopexin-like |
| sequence amino | domain |

| protein | acids total | start | end |
|---|---|---|---|
| SEQ ID NO: 02 | 663 | 469 | 663 |
| SEQ ID NO: 03 | 305 | 97 | 280 |
| SEQ ID NO: 04 | 305 | 97 | 280 |
| SEQ ID NO: 05 | 662 | 468 | 662 |
| SEQ ID NO: 06 | 469 | 275 | 469 |
| SEQ ID NO: 07 | 469 | 275 | 469 |
| SEQ ID NO: 08 | 469 | 275 | 469 |
| SEQ ID NO: 09 | 469 | 275 | 469 |
| SEQ ID NO: 10 | 660 | 466 | 660 |
| SEQ ID NO: 11 | 477 | 287 | 477 |
| SEQ ID NO: 12 | 477 | 287 | 477 |
| SEQ ID NO: 13 | 478 | 288 | 478 |
| SEQ ID NO: 14 | 475 | 285 | 475 |
| SEQ ID NO: 15 | 467 | 276 | 467 |
| SEQ ID NO: 16 | 465 | 276 | 465 |
| SEQ ID NO: 17 | 466 | 277 | 466 |
| SEQ ID NO: 18 | 712 | 518 | 712 |
| SEQ ID NO: 19 | 704 | 510 | 704 |
| SEQ ID NO: 20 | 707 | 513 | 707 |
| SEQ ID NO: 21 | 730 | 531 | 730 |
| SEQ ID NO: 22 | 476 | 286 | 476 |
| SEQ ID NO: 23 | 476 | 286 | 476 |
| SEQ ID NO: 24 | 488 | 291 | 483 |
| SEQ ID NO: 25 | 492 | 295 | 487 |
| SEQ ID NO: 26 | 470 | 279 | 470 |
| SEQ ID NO: 27 | 462 | 272 | 462 |
| SEQ ID NO: 28 | 464 | 274 | 464 |
| SEQ ID NO: 29 | 465 | 275 | 465 |
| SEQ ID NO: 30 | 471 | 281 | 471 |
| SEQ ID NO: 31 | 472 | 282 | 472 |
| SEQ ID NO: 32 | 471 | 281 | 471 |
| SEQ ID NO: 33 | 471 | 281 | 471 |
| SEQ ID NO: 34 | 582 | 316 | 511 |
| SEQ ID NO: 35 | 582 | 316 | 511 |
| SEQ ID NO: 36 | 580 | 314 | 509 |
| SEQ ID NO: 37 | 582 | 316 | 511 |
| SEQ ID NO: 38 | 582 | 316 | 511 |
| SEQ ID NO: 39 | 669 | 367 | 562 |
| SEQ ID NO: 40 | 657 | 365 | 558 |
| SEQ ID NO: 41 | 607 | 340 | 535 |
| SEQ ID NO: 42 | 607 | 340 | 535 |
| SEQ ID NO: 43 | 607 | 340 | 535 |
| SEQ ID NO: 44 | 606 | 332 | 529 |
| SEQ ID NO: 45 | 578 | 333 | 530 |
| SEQ ID NO: 46 | 467 | 277 | 467 |
| SEQ ID NO: 47 | 508 | 286 | 475 |
| SEQ ID NO: 48 | 527 | 286 | 474 |
| SEQ ID NO: 49 | 481 | 291 | 481 |
| SEQ ID NO: 50 | 483 | 293 | 483 |
| SEQ ID NO: 51 | 482 | 292 | 482 |
| SEQ ID NO: 52 | 483 | 293 | 483 |
| SEQ ID NO: 53 | 645 | 377 | 572 |
| SEQ ID NO: 54 | 618 | 350 | 545 |
| SEQ ID NO: 55 | 618 | 350 | 545 |
| SEQ ID NO: 56 | 562 | 314 | 511 |
| SEQ ID NO: 57 | 520 | 328 | 520 |
| SEQ ID NO: 58 | 478 | 288 | 478 |
| SEQ ID NO: 59 | 478 | 287 | 478 |
| SEQ ID NO: 60 | 459 | 265 | 459 |
| SEQ ID NO: 61 | 475 | 288 | 475 |

(Table II end).

A consensus sequence of 210 positions has been determined by the alignment of the above listed hemopexin-like domains (SEQ ID NO:01, SEQ ID NO:88).

The number of different amino acids per position has been determined in order to compile an amino acid diversity number (“determination of variability”; see table III). Gaps in the sequence are marked by a hyphen (−) (see table IV for the alignment of all sequences). For every position of the consensus sequence the number of different amino acids (amino acid diversity number) is given. The maximum possible number is 21 (20 different amino acids+1 gap). A low diversity number indicates a highly conserved position. A high diversity number indicates flexibility at this position.

TABLE III
Amino acid diversity number for each position of the 210 positions of
the consensus sequence; for the consensus sequence without gaps
see SEQ ID NO: 01, for the consensus sequence with gaps see SEQ
ID NO: 88.

| amino acid position in | amino acid |
|---|---|
| consensus sequence | diversity number |
| 1 | 6 |
| 2 | 11 |
| 3 | 12 |
| 4 | 2 |
| 5 | 5 |
| 6 | 7 |
| 7 | 10 |
| 8 | 10 |
| 9 | 11 |
| 10 | 3 |
| 11 | 4 |
| 12 | 6 |
| 13 | 7 |
| 14 | 4 |
| 15 | 10 |
| 16 | 7 |
| 17 | 4 |
| 18 | 7 |
| 19 | 7 |
| 20 | 2 |
| 21 | 8 |
| 22 | 7 |
| 23 | 7 |
| 24 | 2 |
| 25 | 4 |
| 26 | 5 |
| 27 | 9 |
| 28 | 7 |
| 29 | 6 |
| 30 | 2 |
| 31 | 7 |
| 32 | 7 |
| 33 | 8 |
| 34 | 5 |
| 35 | 11 |
| 36 | 10 |
| 37 | 12 |
| 38 | 15 |
| 39 | 11 |
| 40 | 11 |
| 41 | 12 |
| 42 | 9 |
| 43 | 6 |
| 44 | 11 |
| 45 | 7 |
| 46 | 7 |
| 47 | 9 |
| 48 | 12 |
| 49 | 11 |
| 50 | 3 |
| 51 | 7 |
| 52 | 8 |
| 53 | 3 |
| 54 | 2 |
| 55 | 2 |
| 56 | 2 |
| 57 | 2 |
| 58 | 9 |
| 59 | 11 |
| 60 | 4 |
| 61 | 5 |
| 62 | 3 |
| 63 | 2 |
| 64 | 4 |
| 65 | 4 |
| 66 | 10 |
| 67 | 13 |
| 68 | 11 |
| 69 | 9 |
| 70 | 9 |
| 71 | 12 |
| 72 | 7 |
| 73 | 7 |
| 74 | 4 |
| 75 | 4 |
| 76 | 5 |
| 77 | 3 |
| 78 | 10 |
| 79 | 7 |
| 80 | 8 |
| 81 | 4 |
| 82 | 8 |
| 83 | 9 |
| 84 | 12 |
| 85 | 8 |
| 86 | 13 |
| 87 | 11 |
| 88 | 8 |
| 89 | 9 |
| 90 | 11 |
| 81 | 8 |
| 92 | 8 |
| 93 | 5 |
| 94 | 9 |
| 95 | 12 |
| 96 | 11 |
| 97 | 6 |
| 98 | 9 |
| 99 | 8 |
| 100 | 9 |
| 101 | 4 |
| 102 | 9 |
| 103 | 8 |
| 104 | 13 |
| 105 | 11 |
| 106 | 10 |
| 107 | 11 |
| 108 | 10 |
| 109 | 8 |
| 110 | 7 |
| 111 | 5 |
| 112 | 7 |
| 113 | 7 |
| 114 | 9 |
| 115 | 11 |
| 116 | 10 |
| 117 | 13 |
| 118 | 11 |
| 119 | 2 |
| 120 | 1 |
| 121 | 8 |
| 122 | 3 |
| 123 | 9 |
| 124 | 5 |
| 125 | 4 |
| 126 | 4 |
| 127 | 8 |
| 128 | 9 |
| 129 | 12 |
| 130 | 10 |
| 131 | 6 |
| 132 | 3 |
| 133 | 5 |
| 134 | 5 |
| 135 | 6 |
| 136 | 7 |
| 137 | 14 |
| 138 | 11 |
| 139 | 9 |
| 140 | 13 |
| 141 | 8 |
| 142 | 6 |
| 143 | 8 |
| 144 | 10 |
| 145 | 7 |
| 146 | 4 |
| 147 | 8 |
| 148 | 12 |
| 149 | 8 |
| 150 | 8 |
| 151 | 14 |
| 152 | 10 |
| 153 | 9 |
| 154 | 6 |
| 155 | 4 |
| 156 | 4 |
| 157 | 12 |
| 158 | 14 |
| 159 | 11 |
| 160 | 10 |
| 161 | 6 |
| 162 | 4 |
| 163 | 7 |
| 164 | 5 |
| 165 | 3 |
| 166 | 10 |
| 167 | 13 |
| 168 | 12 |
| 169 | 10 |
| 170 | 8 |
| 171 | 8 |
| 172 | 8 |
| 173 | 6 |
| 174 | 5 |
| 175 | 5 |
| 176 | 8 |
| 177 | 3 |
| 178 | 13 |
| 179 | 13 |
| 180 | 5 |
| 181 | 6 |
| 182 | 7 |
| 183 | 6 |
| 184 | 8 |
| 185 | 10 |
| 186 | 12 |
| 187 | 10 |
| 188 | 12 |
| 189 | 5 |
| 190 | 4 |
| 191 | 1 |
| 192 | 3 |
| 193 | 1 |
| 194 | 1 |
| 195 | 2 |
| 196 | 10 |
| 197 | 8 |
| 198 | 7 |
| 199 | 12 |
| 200 | 9 |
| 201 | 10 |
| 202 | 10 |
| 203 | 8 |
| 204 | 8 |
| 205 | 11 |
| 206 | 5 |
| 207 | 4 |
| 208 | 4 |
| 209 | 9 |
| 210 | 1 |

(Table III end).

TABLE IV

Alignment table for the sequences SEQ ID NO: 02
to sequence SEQ ID NO: 61.

| | |
|---|---|
| 1 | 11 |

| | | | | | | | | | | | | | | | | | | | | |
|---|---|---|---|---|---|---|---|---|---|---|---|---|---|---|---|---|---|---|---|---|
| SEQ ID NO: 02 | P | E | L | C | K | H | D | I | V | F | D | G | V | A | Q | I | R | G | E | — |
| SEQ ID NO: 03 | Q | P | P | S | K | N | Q | K | L | L | Y | A | V | C | T | — | I | G | G | R |
| SEQ ID NO: 04 | Q | P | P | S | K | N | Q | K | L | L | Y | A | V | C | T | — | I | G | G | R |
| SEQ ID NO: 05 | P | E | I | C | T | Q | D | I | V | F | D | G | I | A | Q | I | R | G | E | — |
| SEQ ID NO: 06 | P | E | V | C | D | S | K | L | T | F | D | A | I | T | T | I | R | G | E | — |
| SEQ ID NO: 07 | P | K | A | C | D | S | K | L | T | F | D | A | I | T | T | I | R | G | E | — |
| SEQ ID NO: 08 | P | K | A | C | D | S | K | L | T | F | D | A | I | T | T | I | R | G | E | — |
| SEQ ID NO: 09 | P | Q | V | C | D | S | K | L | T | F | D | A | I | T | T | L | R | G | E | — |
| SEQ ID NO: 10 | P | E | I | C | K | Q | D | I | V | F | D | G | I | A | Q | I | R | G | E | — |
| SEQ ID NO: 11 | P | A | N | C | D | P | A | L | S | F | D | A | V | S | T | L | R | G | E | — |
| SEQ ID NO: 12 | S | P | M | C | S | S | T | L | F | F | D | A | V | S | T | L | R | G | E | — |
| SEQ ID NO: 13 | P | V | M | C | D | P | D | L | S | F | D | A | I | S | T | L | R | G | E | — |
| SEQ ID NO: 14 | L | P | M | C | S | S | A | L | S | F | D | A | V | S | T | L | R | G | E | — |
| SEQ ID NO: 15 | P | K | P | C | D | P | S | L | T | F | D | A | I | T | T | L | R | G | E | — |
| SEQ ID NO: 16 | P | K | A | C | D | P | H | L | R | F | D | A | T | T | T | L | R | G | E | — |
| SEQ ID NO: 17 | P | T | A | C | D | P | H | L | R | F | D | A | A | T | T | L | R | G | E | — |
| SEQ ID NO: 18 | E | D | V | C | N | V | D | — | I | F | D | A | I | A | E | I | R | N | R | — |
| SEQ ID NO: 19 | E | D | I | C | K | V | N | — | I | F | D | A | I | A | E | I | R | N | Y | — |
| SEQ ID NO: 20 | D | D | A | C | N | V | N | — | I | F | D | A | I | A | E | I | G | N | Q | — |
| SEQ ID NO: 21 | D | N | P | C | N | V | D | — | V | F | D | A | I | A | E | I | Q | G | A | — |
| SEQ ID NO: 22 | P | A | K | C | D | P | A | L | S | F | D | A | I | S | T | L | R | G | E | — |
| SEQ ID NO: 23 | P | D | K | C | D | P | A | L | S | F | D | S | V | S | T | L | R | G | E | — |
| SEQ ID NO: 24 | P | D | A | C | E | A | — | — | S | F | D | A | V | S | T | I | R | G | E | — |
| SEQ ID NO: 25 | P | D | V | C | E | T | — | — | S | F | D | A | V | S | T | I | R | G | E | — |
| SEQ ID NO: 26 | P | A | L | C | D | P | N | L | S | F | D | A | V | T | T | V | G | N | K | — |
| SEQ ID NO: 27 | S | T | F | C | H | Q | S | L | S | F | D | A | V | T | T | V | G | E | K | — |
| SEQ ID NO: 28 | P | T | A | C | D | H | N | L | K | F | D | A | V | T | T | V | G | N | K | — |
| SEQ ID NO: 29 | S | T | V | C | H | Q | S | L | S | F | D | A | V | T | T | V | G | D | K | — |
| SEQ ID NO: 30 | P | D | K | C | D | P | S | L | S | L | D | A | I | T | S | L | R | G | E | — |
| SEQ ID NO: 31 | P | D | K | C | D | P | S | L | S | L | D | A | I | T | S | L | R | G | E | — |
| SEQ ID NO: 32 | P | D | K | C | D | P | S | L | S | L | D | A | I | T | S | L | R | G | E | — |
| SEQ ID NO: 33 | P | D | K | C | D | P | S | L | S | L | D | A | I | T | S | L | R | G | E | — |
| SEQ ID NO: 34 | P | N | I | C | D | G | N | — | — | F | D | T | V | A | M | L | R | G | E | — |
| SEQ ID NO: 35 | P | N | I | C | D | G | N | — | — | F | D | T | V | A | M | L | R | G | E | — |
| SEQ ID NO: 36 | P | N | I | C | D | G | N | — | — | F | D | T | V | A | M | L | R | G | E | — |
| SEQ ID NO: 37 | P | K | I | C | D | G | N | — | — | F | D | T | V | A | V | F | R | G | E | — |
| SEQ ID NO: 38 | P | N | I | C | D | G | N | — | — | F | D | T | V | A | M | L | R | G | E | — |
| SEQ ID NO: 39 | P | N | I | C | D | G | D | — | — | F | D | T | V | A | M | L | R | G | E | — |
| SEQ ID NO: 40 | — | — | I | C | D | G | N | — | — | F | D | T | V | A | V | L | R | G | E | — |
| SEQ ID NO: 41 | P | N | I | C | D | G | N | — | — | F | N | T | L | A | I | L | R | R | E | — |
| SEQ ID NO: 42 | P | N | I | C | D | G | N | — | — | F | N | T | L | A | I | L | R | R | E | — |
| SEQ ID NO: 43 | P | N | I | C | D | G | N | — | — | F | N | T | L | A | I | L | R | R | E | — |
| SEQ ID NO: 44 | P | H | R | C | S | T | H | — | — | F | D | A | V | A | Q | I | R | G | E | — |
| SEQ ID NO: 45 | P | H | R | C | T | A | H | — | — | F | D | A | V | A | Q | I | R | G | E | — |
| SEQ ID NO: 46 | P | S | R | C | D | P | N | V | V | F | N | A | V | T | T | M | R | G | E | — |
| SEQ ID NO: 47 | P | D | P | C | S | S | E | L | D | A | M | M | L | G | — | P | R | G | K | — |
| SEQ ID NO: 48 | P | N | P | C | S | G | E | V | D | A | M | V | L | G | — | P | R | G | K | — |
| SEQ ID NO: 49 | P | D | L | C | D | S | N | L | S | F | D | A | V | T | M | L | G | K | E | — |
| SEQ ID NO: 50 | P | D | L | C | D | S | S | S | S | F | D | A | V | T | M | L | G | K | E | — |
| SEQ ID NO: 51 | P | D | L | C | D | S | S | S | S | F | D | A | V | T | M | L | G | K | E | — |
| SEQ ID NO: 52 | P | D | I | C | D | S | S | S | S | F | D | A | V | T | M | L | G | K | E | — |
| SEQ ID NO: 53 | P | N | I | C | D | G | N | — | — | F | N | T | V | A | L | F | R | G | E | — |
| SEQ ID NO: 54 | P | N | I | C | D | G | N | — | — | F | N | T | V | A | L | F | R | G | E | — |
| SEQ ID NO: 55 | P | N | I | C | D | G | N | — | — | F | N | T | V | A | L | F | R | G | E | — |
| SEQ ID NO: 56 | P | D | R | C | E | G | N | — | — | F | D | A | I | A | N | I | R | G | E | — |
| SEQ ID NO: 57 | — | — | — | — | — | — | — | — | — | F | D | A | I | T | V | D | R | Q | Q | Q |
| SEQ ID NO: 58 | Q | E | E | C | E | G | S | S | V | F | E | H | F | A | M | M | Q | R | D | — |
| SEQ ID NO: 59 | Q | E | E | C | E | G | S | S | V | F | E | H | F | A | L | L | Q | R | D | — |
| SEQ ID NO: 60 | R | E | E | C | E | G | S | S | V | F | A | H | F | A | L | M | Q | R | D | — |
| SEQ ID NO: 61 | Q | E | E | C | E | G | S | S | V | F | E | H | F | A | M | L | H | R | D | — |

| | |
|---|---|
| 21 | 31 |

| | | | | | | | | | | | | | | | | | | | | |
|---|---|---|---|---|---|---|---|---|---|---|---|---|---|---|---|---|---|---|---|---|
| SEQ ID NO: 02 | I | F | F | F | K | D | R | F | M | W | R | T | — | V | N | P | R | G | K | P |
| SEQ ID NO: 03 | — | — | W | L | E | E | R | C | Y | D | L | F | R | N | R | — | — | — | — | — |
| SEQ ID NO: 04 | — | — | W | L | E | E | R | C | Y | D | L | F | R | N | R | — | — | — | — | — |
| SEQ ID NO: 05 | I | F | F | F | K | D | R | F | I | W | R | T | — | V | T | P | G | D | K | P |
| SEQ ID NO: 06 | V | M | F | F | K | U | N | F | Y | M | R | T | — | N | P | L | Y | P | E | — |
| SEQ ID NO: 07 | V | M | F | F | K | D | R | F | Y | M | R | I | — | N | P | Y | Y | P | E | — |
| SEQ ID NO: 08 | V | M | F | F | K | D | R | F | Y | M | R | T | — | N | P | F | Y | P | E | — |
| SEQ ID NO: 09 | L | M | F | F | K | D | R | F | Y | M | R | T | — | N | S | F | Y | P | E | — |
| SEQ ID NO: 10 | I | F | F | F | K | D | R | F | I | W | R | T | — | V | T | P | R | D | K | P |
| SEQ ID NO: 11 | I | L | I | F | K | D | R | H | F | W | R | K | — | S | L | R | K | L | E | — |
| SEQ ID NO: 12 | V | L | F | F | K | D | R | H | F | W | R | K | — | S | L | R | T | P | E | — |
| SEQ ID NO: 13 | I | L | F | F | K | D | R | Y | F | W | R | K | — | S | L | R | I | L | E | — |
| SEQ ID NO: 14 | V | L | F | F | K | D | R | H | F | W | R | K | — | S | L | R | T | P | E | — |
| SEQ ID NO: 15 | I | L | F | F | K | D | R | Y | F | W | R | R | — | H | P | Q | L | Q | R | — |
| SEQ ID NO: 16 | I | Y | F | F | K | E | K | Y | F | W | R | R | — | H | P | Q | L | R | T | — |
| SEQ ID NO: 17 | I | Y | F | F | K | D | K | Y | F | W | R | R | — | H | P | Q | L | R | T | — |
| SEQ ID NO: 18 | L | H | F | F | K | A | G | K | Y | W | R | L | S | E | G | G | G | R | R | V |
| SEQ ID NO: 19 | L | H | F | F | K | E | G | K | Y | W | R | F | S | K | G | K | G | R | R | V |
| SEQ ID NO: 20 | L | Y | L | F | K | D | G | K | Y | W | R | F | S | E | G | R | G | S | R | P |
| SEQ ID NO: 21 | L | H | F | F | K | D | G | W | Y | W | K | F | L | N | H | R | G | S | P | L |
| SEQ ID NO: 22 | Y | L | F | F | K | D | R | Y | F | W | R | R | — | S | H | W | N | P | E | — |
| SEQ ID NO: 23 | V | L | F | F | K | D | R | Y | F | W | R | R | — | S | H | W | N | P | E | — |
| SEQ ID NO: 24 | L | F | F | F | K | A | G | F | V | W | R | L | R | G | G | Q | — | L | Q | P |
| SEQ ID NO: 25 | L | F | F | F | K | A | G | F | V | W | R | L | R | S | G | R | — | L | Q | P |
| SEQ ID NO: 26 | I | F | F | F | K | D | R | F | F | W | L | K | — | V | S | E | R | P | K | — |
| SEQ ID NO: 27 | I | L | F | F | K | D | W | F | F | W | W | K | — | L | P | G | S | P | A | — |
| SEQ ID NO: 28 | I | F | F | F | K | D | S | F | F | W | W | K | — | I | P | K | S | S | T | — |
| SEQ ID NO: 29 | I | F | F | F | K | D | W | F | F | W | W | R | — | L | P | G | S | P | A | — |
| SEQ ID NO: 30 | T | L | I | F | K | D | R | F | F | W | R | L | — | H | P | Q | Q | V | E | — |
| SEQ ID NO: 31 | T | M | V | F | K | D | R | F | F | W | R | L | — | H | P | Q | L | V | D | — |
| SEQ ID NO: 32 | T | M | I | F | K | D | R | F | F | W | R | L | — | H | P | Q | Q | V | D | — |
| SEQ ID NO: 33 | T | M | I | F | K | D | R | F | F | W | R | L | — | H | P | Q | Q | V | D | — |
| SEQ ID NO: 34 | M | F | V | F | K | K | R | W | F | W | R | V | R | N | N | Q | — | V | M | D |
| SEQ ID NO: 35 | M | F | V | F | K | E | R | W | F | W | R | V | R | N | N | Q | — | V | M | D |
| SEQ ID NO: 36 | M | F | V | F | K | E | R | W | F | W | R | V | R | K | N | Q | — | V | M | D |
| SEQ ID NO: 37 | M | F | V | F | K | E | R | W | F | W | R | V | R | N | N | Q | — | V | M | D |
| SEQ ID NO: 38 | M | F | V | F | K | E | R | W | F | W | R | V | R | N | N | Q | — | V | M | D |
| SEQ ID NO: 39 | M | F | V | F | K | G | R | W | F | W | R | V | R | H | N | R | — | V | L | D |
| SEQ ID NO: 40 | M | F | V | F | K | G | R | W | F | W | R | V | R | H | N | R | — | V | L | D |
| SEQ ID NO: 41 | M | F | V | F | K | D | Q | W | F | W | R | V | R | N | N | R | — | V | M | D |
| SEQ ID NO: 42 | M | F | V | F | K | D | Q | W | F | W | R | V | R | N | N | R | — | V | M | D |
| SEQ ID NO: 43 | M | F | V | F | K | D | Q | W | F | W | R | V | R | N | N | R | — | V | M | D |
| SEQ ID NO: 44 | A | F | F | F | K | G | K | Y | F | W | R | L | T | R | D | R | H | L | V | S |
| SEQ ID NO: 45 | A | F | F | F | K | G | K | Y | F | W | R | L | T | R | D | R | H | L | V | S |
| SEQ ID NO: 46 | L | I | F | F | V | K | R | F | L | W | R | K | — | H | P | Q | A | S | E | — |
| SEQ ID NO: 47 | T | Y | A | F | K | G | D | Y | V | W | T | V | S | D | S | G | P | G | P | — |
| SEQ ID NO: 48 | T | Y | A | F | K | G | D | Y | V | W | T | V | T | D | S | G | P | G | P | — |
| SEQ ID NO: 49 | L | L | L | F | R | D | R | I | F | W | R | R | — | Q | V | H | L | M | S | G |
| SEQ ID NO: 50 | L | L | L | F | K | D | R | I | F | W | R | R | — | Q | V | H | L | R | T | G |
| SEQ ID NO: 51 | L | L | F | F | K | D | R | I | F | W | R | R | — | Q | V | H | L | P | T | G |
| SEQ ID NO: 52 | L | L | F | F | R | D | R | I | F | W | R | R | — | Q | V | H | L | M | S | G |
| SEQ ID NO: 53 | M | F | V | F | K | D | R | W | F | W | R | L | R | N | N | R | — | V | Q | E |
| SEQ ID NO: 54 | M | F | V | F | K | D | R | W | F | W | R | L | R | N | N | R | — | V | Q | E |
| SEQ ID NO: 55 | M | F | V | F | K | D | R | W | F | W | R | L | R | N | N | R | — | V | Q | E |
| SEQ ID NO: 56 | T | F | F | F | K | G | P | W | F | W | R | L | Q | P | S | G | Q | L | V | S |
| SEQ ID NO: 57 | L | Y | I | F | K | G | S | H | F | W | E | V | A | A | D | G | N | V | S | — |
| SEQ ID NO: 58 | S | W | E | D | I | F | E | L | L | F | W | G | R | T | S | A | G | T | R | — |
| SEQ ID NO: 59 | S | W | E | N | I | F | E | L | L | F | W | G | R | S | S | D | G | A | R | — |
| SEQ ID NO: 60 | S | W | E | D | I | F | R | L | L | F | W | S | H | S | F | G | G | A | I | — |
| SEQ ID NO: 61 | S | W | E | D | I | F | K | L | L | F | W | G | R | P | S | G | G | A | R | — |

| | |
|---|---|
| 41 | 51 |

| | | | | | | | | | | | | | | | | | | | | |
|---|---|---|---|---|---|---|---|---|---|---|---|---|---|---|---|---|---|---|---|---|
| SEQ ID NO: 02 | T | G | P | L | L | V | A | T | F | W | P | D | L | P | E | — | — | — | K | I |
| SEQ ID NO: 03 | — | H | L | A | S | F | G | K | A | K | Q | C | M | N | F | V | V | G | — | — |
| SEQ ID NO: 04 | — | H | L | A | S | F | G | K | A | K | Q | C | M | N | F | V | V | G | — | — |
| SEQ ID NO: 05 | M | G | P | L | L | V | A | T | F | W | P | E | L | P | E | — | — | — | K | I |
| SEQ ID NO: 06 | V | E | L | N | F | I | S | V | F | W | P | Q | L | P | N | — | — | — | G | L |
| SEQ ID NO: 07 | A | E | L | N | F | I | S | I | F | W | P | Q | L | P | N | — | — | — | G | L |
| SEQ ID NO: 08 | V | E | L | N | F | I | S | V | F | W | P | Q | L | P | N | — | — | — | G | L |
| SEQ ID NO: 09 | V | E | L | N | F | I | S | V | F | W | P | Q | V | P | N | — | — | — | G | L |
| SEQ ID NO: 10 | M | G | P | L | L | V | A | T | F | W | P | E | L | P | E | — | — | — | K | I |
| SEQ ID NO: 11 | P | E | L | H | L | I | S | S | F | W | P | S | L | P | S | — | — | — | G | V |
| SEQ ID NO: 12 | P | E | F | Y | L | I | S | S | F | W | P | S | L | P | S | — | — | — | N | M |
| SEQ ID NO: 13 | P | E | F | H | L | I | S | S | F | W | P | S | L | P | S | — | — | — | A | V |
| SEQ ID NO: 14 | P | G | F | Y | L | I | S | S | F | W | P | S | L | P | S | — | — | — | N | M |
| SEQ ID NO: 15 | V | E | M | N | F | I | S | L | F | W | P | S | L | P | T | — | — | — | G | I |
| SEQ ID NO: 16 | V | D | L | N | F | I | S | L | F | W | P | G | L | P | N | — | — | — | G | L |
| SEQ ID NO: 17 | V | D | L | N | F | I | S | L | F | W | P | F | L | P | N | — | — | — | G | L |
| SEQ ID NO: 18 | Q | G | P | F | L | V | K | S | K | W | P | A | L | P | R | — | — | — | K | L |
| SEQ ID NO: 19 | Q | G | P | F | L | S | P | S | T | W | P | A | L | P | R | — | — | — | K | L |
| SEQ ID NO: 20 | Q | G | P | F | L | I | A | D | K | W | P | A | L | P | R | — | — | — | K | L |
| SEQ ID NO: 21 | Q | G | P | F | L | T | A | R | T | W | P | A | L | P | A | — | — | — | T | L |
| SEQ ID NO: 22 | P | E | F | H | L | I | S | A | F | W | P | S | L | P | S | — | — | — | Y | L |
| SEQ ID NO: 23 | P | E | F | H | L | I | S | A | F | W | P | T | L | P | S | — | — | — | D | L |
| SEQ ID NO: 24 | G | Y | P | A | L | A | S | R | H | W | Q | G | L | P | S | — | — | — | P | V |
| SEQ ID NO: 25 | G | Y | P | A | L | A | S | R | H | W | Q | G | L | P | S | — | — | — | P | V |
| SEQ ID NO: 26 | T | S | V | N | I | I | S | S | L | W | P | T | L | P | S | — | — | — | G | I |
| SEQ ID NO: 27 | T | N | I | T | S | I | S | S | I | W | P | S | I | P | S | — | — | — | A | I |
| SEQ ID NO: 28 | T | S | V | R | L | I | S | S | L | W | P | T | L | P | S | — | — | — | G | I |
| SEQ ID NO: 29 | T | N | I | T | S | I | S | S | M | W | P | T | I | P | S | — | — | — | G | I |
| SEQ ID NO: 30 | A | E | L | F | L | T | K | S | F | G | P | E | L | P | N | — | — | — | R | I |
| SEQ ID NO: 31 | A | E | L | F | L | T | K | S | F | W | P | E | L | P | N | — | — | — | R | I |
| SEQ ID NO: 32 | A | E | L | F | L | T | K | S | F | W | P | E | L | P | N | — | — | — | R | I |
| SEQ ID NO: 33 | A | E | L | F | L | T | K | S | F | W | P | E | L | P | N | — | — | — | R | I |
| SEQ ID NO: 34 | G | Y | P | M | P | I | G | Q | F | W | R | G | L | P | A | — | — | — | S | I |
| SEQ ID NO: 35 | G | Y | P | M | P | I | G | Q | F | W | R | G | L | P | A | — | — | — | S | I |
| SEQ ID NO: 36 | G | Y | P | M | P | I | G | Q | F | W | R | G | L | P | A | — | — | — | S | I |
| SEQ ID NO: 37 | G | Y | P | M | P | I | G | Q | L | W | R | G | L | P | A | — | — | — | S | I |
| SEQ ID NO: 38 | G | Y | P | M | P | I | G | Q | F | W | R | G | L | P | A | — | — | — | S | I |
| SEQ ID NO: 39 | N | Y | P | M | P | I | G | H | F | W | R | G | L | P | G | — | — | — | D | I |
| SEQ ID NO: 40 | N | Y | P | M | P | I | G | H | F | W | R | G | L | P | G | — | — | — | N | I |
| SEQ ID NO: 41 | G | Y | P | M | Q | I | T | Y | F | W | R | G | L | P | P | — | — | — | S | I |
| SEQ ID NO: 42 | G | Y | P | M | Q | I | T | Y | F | W | R | G | L | P | P | — | — | — | S | I |
| SEQ ID NO: 43 | G | Y | P | M | Q | I | T | Y | F | W | R | G | L | P | P | — | — | — | S | I |
| SEQ ID NO: 44 | L | Q | P | A | Q | M | H | R | F | W | R | G | L | P | L | H | L | D | S | V |
| SEQ ID NO: 45 | L | Q | P | A | Q | M | H | R | F | W | R | G | L | P | L | H | L | D | S | V |
| SEQ ID NO: 46 | A | E | L | M | F | V | Q | A | F | W | P | S | L | P | T | — | — | — | N | I |
| SEQ ID NO: 47 | — | — | L | F | R | V | S | A | L | W | E | G | L | P | G | — | — | — | N | L |
| SEQ ID NO: 48 | — | — | L | F | Q | I | S | A | L | W | E | G | L | P | G | — | — | — | N | L |
| SEQ ID NO: 49 | I | R | P | S | T | I | T | S | S | F | P | Q | L | M | S | — | — | — | N | V |
| SEQ ID NO: 50 | I | R | P | S | T | I | T | S | S | F | P | Q | L | M | S | — | — | — | N | V |
| SEQ ID NO: 51 | I | R | P | S | T | I | T | S | S | F | P | Q | L | M | S | — | — | — | N | V |
| SEQ ID NO: 52 | I | R | P | S | T | I | T | S | S | F | P | Q | L | M | S | — | — | — | N | V |
| SEQ ID NO: 53 | G | Y | P | M | Q | I | E | Q | F | W | K | G | L | P | A | — | — | — | R | I |
| SEQ ID NO: 54 | G | Y | P | M | Q | I | E | Q | F | W | K | G | L | P | A | — | — | — | R | I |
| SEQ ID NO: 55 | G | Y | P | M | Q | I | E | Q | F | W | K | G | L | P | A | — | — | — | R | I |
| SEQ ID NO: 56 | P | R | P | A | R | L | H | R | F | W | E | G | L | P | A | Q | V | R | V | V |
| SEQ ID NO: 57 | — | E | P | R | P | L | Q | E | R | W | V | G | L | P | P | — | — | — | N | I |
| SEQ ID NO: 58 | — | Q | P | Q | F | I | S | R | D | W | H | G | V | P | G | — | — | — | — | — |
| SEQ ID NO: 59 | — | E | P | Q | F | I | S | R | N | W | H | G | V | P | G | — | — | — | — | — |
| SEQ ID NO: 60 | — | E | P | R | V | I | S | Q | D | W | L | G | L | P | E | — | — | — | — | — |
| SEQ ID NO: 61 | — | Q | P | Q | F | I | S | R | D | W | H | G | V | P | G | — | — | — | — | — |

| | |
|---|---|
| 61 | 71 |

| | | | | | | | | | | | | | | | | | | | | |
|---|---|---|---|---|---|---|---|---|---|---|---|---|---|---|---|---|---|---|---|---|
| SEQ ID NO: 02 | D | A | V | Y | E | S | P | Q | D | E | K | A | V | F | F | A | G | N | E | Y |
| SEQ ID NO: 03 | — | — | — | — | — | — | — | — | — | — | L | L | K | L | G | E | L | M | N | F |
| SEQ ID NO: 04 | — | — | — | — | — | — | — | — | — | — | L | L | K | L | G | E | L | M | N | F |
| SEQ ID NO: 05 | D | A | V | Y | E | A | P | Q | E | E | K | A | V | F | F | A | G | N | E | Y |
| SEQ ID NO: 06 | D | A | A | Y | E | V | A | D | R | D | E | V | R | F | F | K | G | N | K | Y |
| SEQ ID NO: 07 | D | A | A | Y | E | V | S | H | R | D | E | V | R | F | F | K | G | N | K | Y |
| SEQ ID NO: 08 | E | A | A | Y | E | F | A | D | R | D | E | V | R | F | F | K | G | N | K | Y |
| SEQ ID NO: 09 | Q | A | A | Y | E | I | A | D | R | D | E | V | R | F | F | K | G | N | K | Y |
| SEQ ID NO: 10 | D | A | V | Y | E | A | P | Q | E | E | K | A | V | F | F | A | G | N | E | Y |
| SEQ ID NO: 11 | D | A | A | Y | E | V | T | S | K | D | L | V | F | I | F | K | G | N | Q | F |
| SEQ ID NO: 12 | D | A | A | Y | E | V | T | N | R | D | T | V | F | I | F | K | G | N | Q | F |
| SEQ ID NO: 13 | D | A | A | Y | E | V | I | S | R | D | T | V | F | I | F | K | G | T | Q | F |
| SEQ ID NO: 14 | D | A | A | Y | E | V | T | N | R | D | T | V | F | I | L | K | G | N | Q | I |
| SEQ ID NO: 15 | Q | A | A | Y | E | D | F | D | R | D | L | I | F | L | F | K | G | N | Q | Y |
| SEQ ID NO: 16 | Q | A | A | Y | E | D | F | D | R | D | L | V | F | L | F | K | G | R | Q | Y |
| SEQ ID NO: 17 | Q | A | A | Y | E | D | F | D | R | D | L | V | F | L | F | K | G | R | Q | Y |
| SEQ ID NO: 18 | D | S | A | F | E | D | P | L | T | K | K | I | F | F | F | S | G | R | Q | V |
| SEQ ID NO: 19 | D | S | A | F | E | D | G | L | T | K | K | T | F | F | F | S | G | R | Q | V |
| SEQ ID NO: 20 | D | S | V | F | E | E | P | L | S | K | K | L | F | F | F | S | G | R | Q | V |
| SEQ ID NO: 21 | D | S | A | F | E | D | P | Q | T | K | R | V | F | F | F | S | G | R | Q | M |
| SEQ ID NO: 22 | D | A | A | Y | E | V | N | S | R | D | T | V | F | I | F | K | G | N | E | F |
| SEQ ID NO: 23 | D | A | A | Y | E | A | H | N | T | D | S | V | L | I | F | K | G | S | Q | F |
| SEQ ID NO: 24 | D | A | A | F | E | D | A | Q | G | — | H | I | W | F | F | Q | G | A | Q | Y |
| SEQ ID NO: 25 | D | A | A | F | E | D | A | Q | G | — | Q | I | W | F | F | Q | G | A | Q | Y |
| SEQ ID NO: 26 | E | A | A | Y | E | I | E | A | R | N | Q | V | F | L | F | K | D | D | K | Y |
| SEQ ID NO: 27 | Q | A | A | Y | E | I | E | S | R | N | Q | L | F | L | F | K | D | E | K | Y |
| SEQ ID NO: 28 | E | A | A | Y | E | I | G | D | R | H | Q | V | F | L | F | K | G | D | K | F |
| SEQ ID NO: 29 | Q | A | A | Y | E | I | G | G | R | N | Q | L | F | L | F | K | D | E | K | Y |
| SEQ ID NO: 30 | D | A | A | Y | E | H | P | S | H | D | L | I | F | I | F | R | G | R | K | F |
| SEQ ID NO: 31 | D | A | A | Y | E | H | P | S | K | D | L | I | F | I | F | R | G | R | K | F |
| SEQ ID NO: 32 | D | A | A | Y | E | H | P | S | H | D | L | I | F | I | F | R | G | R | K | F |
| SEQ ID NO: 33 | D | A | A | Y | E | H | P | A | R | D | L | I | F | I | F | R | G | K | K | F |
| SEQ ID NO: 34 | N | T | A | Y | E | R | K | — | D | G | K | F | V | F | F | K | G | D | K | H |
| SEQ ID NO: 35 | N | T | A | Y | E | R | K | — | D | G | K | F | V | F | F | K | G | D | K | H |
| SEQ ID NO: 36 | N | T | A | Y | E | R | K | — | D | G | K | F | V | F | F | K | G | D | K | H |
| SEQ ID NO: 37 | N | T | A | Y | E | R | K | — | D | G | K | F | V | F | F | K | G | D | K | H |
| SEQ ID NO: 38 | N | T | A | Y | E | R | K | — | D | G | K | F | V | F | F | K | G | D | K | H |
| SEQ ID NO: 39 | S | A | A | Y | E | R | Q | — | D | G | R | F | V | F | F | K | G | D | R | Y |
| SEQ ID NO: 40 | S | A | A | Y | E | R | Q | — | D | G | H | F | V | F | F | K | G | N | R | Y |
| SEQ ID NO: 41 | D | A | V | Y | E | N | S | — | D | G | N | F | V | F | F | K | G | N | K | Y |
| SEQ ID NO: 42 | D | A | V | Y | E | N | S | — | D | G | N | F | V | F | F | K | G | N | K | Y |
| SEQ ID NO: 43 | D | A | V | Y | E | N | S | — | D | G | N | F | V | F | F | K | G | N | K | Y |
| SEQ ID NO: 44 | D | A | V | Y | E | R | T | S | D | H | K | I | V | F | F | K | G | D | R | Y |
| SEQ ID NO: 45 | D | A | V | Y | E | R | T | S | D | H | K | I | V | F | F | K | G | D | R | Y |
| SEQ ID NO: 46 | D | A | A | Y | E | N | P | I | T | E | Q | I | L | V | F | K | G | S | K | Y |
| SEQ ID NO: 47 | D | A | A | V | Y | S | P | R | T | Q | W | I | H | F | F | K | G | D | K | V |
| SEQ ID NO: 48 | D | A | A | V | Y | S | P | R | T | R | R | T | H | F | F | K | G | N | K | V |
| SEQ ID NO: 49 | D | A | A | Y | E | V | A | E | R | G | T | A | Y | F | F | K | G | P | H | Y |
| SEQ ID NO: 50 | D | A | A | Y | E | V | A | E | R | G | T | A | Y | F | F | K | G | P | H | Y |
| SEQ ID NO: 51 | D | A | A | Y | E | V | A | E | R | G | I | A | F | F | F | K | G | P | H | Y |
| SEQ ID NO: 52 | D | A | A | Y | E | V | A | D | R | G | M | A | Y | F | F | K | G | P | H | Y |
| SEQ ID NO: 53 | D | A | A | Y | E | R | A | — | D | G | R | F | V | F | F | K | G | D | K | Y |
| SEQ ID NO: 54 | D | A | A | Y | E | R | A | — | D | G | R | F | V | F | F | K | G | D | K | Y |
| SEQ ID NO: 55 | D | A | A | Y | E | R | A | — | D | G | R | F | V | F | F | K | G | D | K | Y |
| SEQ ID NO: 56 | Q | A | A | Y | A | R | H | R | D | G | R | I | L | L | F | S | G | P | Q | F |
| SEQ ID NO: 57 | E | A | A | A | V | S | L | N | D | G | D | F | Y | F | F | K | G | G | R | C |
| SEQ ID NO: 58 | — | — | Q | V | D | A | A | M | A | G | R | I | Y | I | S | G | M | A | P | R |
| SEQ ID NO: 59 | — | — | K | V | D | A | A | M | A | G | R | I | Y | V | T | G | S | L | S | H |
| SEQ ID NO: 60 | — | — | Q | V | D | A | A | M | A | G | Q | I | Y | I | S | G | S | A | L | K |
| SEQ ID NO: 61 | — | — | K | V | D | A | A | M | A | G | R | I | Y | I | S | G | L | T | P | S |

| | |
|---|---|
| 81 | 91 |

| | | | | | | | | | | | | | | | | | | | | |
|---|---|---|---|---|---|---|---|---|---|---|---|---|---|---|---|---|---|---|---|---|
| SEQ ID NO: 02 | W | V | Y | T | A | S | N | L | D | R | G | Y | P | K | K | L | T | — | S | L |
| SEQ ID NO: 03 | L | I | F | L | Q | K | G | K | F | A | T | L | T | E | R | L | L | G | I | H |
| SEQ ID NO: 04 | L | I | F | L | Q | K | G | K | F | A | T | L | T | E | R | L | L | G | I | H |
| SEQ ID NO: 05 | W | V | Y | S | A | S | T | L | E | R | G | Y | P | K | P | L | T | — | S | L |
| SEQ ID NO: 06 | W | A | V | K | G | Q | D | V | L | R | G | Y | P | R | D | I | Y | R | S | F |
| SEQ ID NO: 07 | W | A | V | K | G | Q | D | V | L | Y | G | Y | P | K | D | I | H | R | S | F |
| SEQ ID NO: 08 | W | A | V | Q | G | Q | N | V | L | H | G | Y | P | K | D | I | Y | S | S | F |
| SEQ ID NO: 09 | W | A | V | R | G | Q | D | V | L | Y | G | Y | P | K | D | I | H | R | S | F |
| SEQ ID NO: 10 | W | I | Y | S | A | S | T | L | E | R | G | Y | P | K | P | L | T | — | S | L |
| SEQ ID NO: 11 | W | A | I | R | G | N | E | V | R | A | G | Y | P | R | G | I | H | — | T | L |
| SEQ ID NO: 12 | W | A | I | R | G | H | E | E | L | A | G | Y | P | K | S | I | H | — | T | L |
| SEQ ID NO: 13 | W | A | I | R | G | N | E | V | Q | A | G | Y | P | R | S | I | H | — | T | L |
| SEQ ID NO: 14 | W | A | I | R | G | H | E | E | L | A | G | Y | P | K | S | I | H | — | T | L |
| SEQ ID NO: 15 | W | A | L | S | G | Y | D | I | L | Q | G | Y | P | K | D | I | S | — | N | Y |
| SEQ ID NO: 16 | W | A | L | S | G | Y | D | L | Q | Q | G | Y | P | R | D | I | S | — | N | Y |
| SEQ ID NO: 17 | W | A | L | S | A | Y | D | L | Q | Q | G | Y | P | R | D | I | S | — | N | Y |
| SEQ ID NO: 18 | W | V | Y | T | G | — | A | S | L | L | G | — | P | R | R | L | D | — | K | L |
| SEQ ID NO: 19 | W | V | Y | T | G | — | T | S | V | V | G | — | P | R | R | L | D | — | K | L |
| SEQ ID NO: 20 | W | V | Y | T | G | — | A | S | V | L | G | — | P | R | R | L | D | — | K | L |
| SEQ ID NO: 21 | W | V | Y | T | G | — | K | T | V | L | G | — | P | R | S | L | D | — | K | L |
| SEQ ID NO: 22 | W | A | I | R | G | N | E | V | Q | A | G | Y | P | R | G | I | H | — | T | L |
| SEQ ID NO: 23 | W | A | V | R | G | N | E | V | Q | A | G | Y | P | K | G | I | H | — | T | L |
| SEQ ID NO: 24 | W | V | Y | D | G | E | K | P | V | L | G | — | P | A | P | L | T | — | E | L |
| SEQ ID NO: 25 | W | V | Y | D | G | E | K | P | V | L | G | — | P | A | P | L | S | — | K | L |
| SEQ ID NO: 26 | W | L | I | S | N | L | R | P | E | P | N | Y | P | K | S | I | H | — | S | F |
| SEQ ID NO: 27 | W | L | I | N | N | L | V | P | E | P | H | Y | P | R | S | I | Y | — | S | L |
| SEQ ID NO: 28 | W | L | I | S | H | L | R | L | Q | P | N | Y | P | K | S | I | H | — | S | L |
| SEQ ID NO: 29 | W | L | I | N | N | L | V | P | E | P | H | Y | P | R | S | I | H | — | S | L |
| SEQ ID NO: 30 | W | A | L | S | G | Y | D | I | L | E | D | Y | P | K | K | I | S | — | E | L |
| SEQ ID NO: 31 | W | A | L | N | G | Y | D | I | L | E | G | Y | P | Q | K | I | S | — | E | L |
| SEQ ID NO: 32 | W | A | L | N | G | Y | D | I | L | E | G | Y | P | K | K | I | S | — | E | L |
| SEQ ID NO: 33 | W | A | P | N | G | Y | D | I | L | E | G | Y | P | Q | K | L | S | — | E | L |
| SEQ ID NO: 34 | W | V | F | D | E | A | S | L | E | P | G | Y | P | K | H | I | K | E | L | G |
| SEQ ID NO: 35 | W | V | F | D | E | A | S | L | E | P | G | Y | P | K | H | I | K | E | L | G |
| SEQ ID NO: 36 | W | V | F | D | E | A | S | L | E | P | G | Y | P | K | H | I | K | E | L | G |
| SEQ ID NO: 37 | W | V | F | D | E | A | S | L | E | P | G | Y | P | K | H | I | K | E | L | G |
| SEQ ID NO: 38 | W | V | F | D | E | A | S | L | E | P | G | Y | P | K | H | I | K | E | L | G |
| SEQ ID NO: 39 | W | L | F | R | E | A | N | L | E | P | G | Y | P | Q | P | L | T | S | Y | G |
| SEQ ID NO: 40 | W | L | F | R | E | A | N | L | E | P | G | Y | P | Q | P | L | S | S | Y | G |
| SEQ ID NO: 41 | W | V | F | K | D | T | T | L | Q | P | G | Y | P | H | D | L | I | T | L | G |
| SEQ ID NO: 42 | W | V | F | K | D | T | T | L | Q | P | G | Y | P | H | D | L | I | T | L | G |
| SEQ ID NO: 43 | W | V | F | K | D | T | T | L | Q | P | G | Y | P | H | D | L | I | T | L | G |
| SEQ ID NO: 44 | W | V | F | K | D | N | N | V | E | E | G | Y | P | R | P | V | S | D | F | S |
| SEQ ID NO: 45 | W | V | P | K | D | N | N | V | E | E | G | Y | P | R | P | V | S | D | P | S |
| SEQ ID NO: 46 | T | A | L | D | G | F | D | V | V | Q | G | Y | P | R | N | I | Y | — | S | L |
| SEQ ID NO: 47 | W | R | Y | I | N | F | K | M | S | P | G | F | P | K | K | L | N | — | — | — |
| SEQ ID NO: 48 | W | R | Y | V | D | F | K | M | S | P | G | F | P | M | K | F | N | — | — | — |
| SEQ ID NO: 49 | W | I | T | R | G | F | Q | M | Q | — | G | P | P | R | T | I | Y | — | D | F |
| SEQ ID NO: 50 | W | I | T | R | G | F | Q | M | Q | — | G | P | P | R | T | I | Y | — | D | F |
| SEQ ID NO: 51 | W | V | T | R | G | P | H | M | Q | — | G | P | P | R | T | I | Y | — | D | F |
| SEQ ID NO: 52 | W | I | T | R | G | F | Q | M | Q | — | G | P | P | R | T | I | Y | — | D | F |
| SEQ ID NO: 53 | W | V | F | K | E | V | T | V | E | P | G | Y | P | H | S | L | G | E | L | G |
| SEQ ID NO: 54 | W | V | F | K | E | V | T | V | E | P | G | Y | P | H | S | L | G | E | L | G |
| SEQ ID NO: 55 | W | V | F | K | E | V | T | V | E | P | G | Y | P | H | S | L | G | E | L | G |
| SEQ ID NO: 56 | W | V | F | Q | D | R | Q | L | E | G | G | — | A | R | P | L | T | E | L | G |
| SEQ ID NO: 57 | W | R | F | R | Q | P | K | P | V | W | G | L | P | Q | L | C | R | — | — | — |
| SEQ ID NO: 58 | P | S | L | A | K | K | Q | R | F | R | H | R | N | R | K | G | Y | R | S | Q |
| SEQ ID NO: 59 | S | A | Q | A | K | K | Q | K | S | K | R | R | S | R | K | R | V | R | S | R |
| SEQ ID NO: 60 | P | S | Q | P | K | M | T | K | S | A | R | R | S | G | K | R | Y | R | S | R |
| SEQ ID NO: 61 | P | S | — | A | K | K | Q | K | S | R | R | R | S | R | K | R | Y | R | S | R |

| | |
|---|---|
| 101 | 111 |

| | | | | | | | | | | | | | | | | | | |
|---|---|---|---|---|---|---|---|---|---|---|---|---|---|---|---|---|---|---|
| SEQ ID NO: 02 | G | L | P | P | D | V | Q | R | I | D | A | A | F | N | W | G | R | N |
| SEQ ID NO: 03 | S | V | F | C | K | P | Q | N | M | R | E | V | G | F | E | Y | M | N |
| SEQ ID NO: 04 | S | V | F | C | K | P | Q | S | M | R | E | V | G | F | E | Y | M | N |
| SEQ ID NO: 05 | G | L | P | P | D | V | Q | R | V | D | A | A | F | N | W | S | K | N |
| SEQ ID NO: 06 | G | F | P | R | T | V | K | S | I | D | A | A | V | S | E | E | D | T |
| SEQ ID NO: 07 | G | F | P | S | T | V | K | N | I | D | A | A | V | S | E | E | D | T |
| SEQ ID NO: 08 | G | F | P | R | T | V | K | H | I | D | A | A | L | S | E | E | N | T |
| SEQ ID NO: 09 | G | F | P | S | T | V | K | N | I | D | A | A | V | F | E | E | D | T |
| SEQ ID NO: 10 | G | L | P | P | D | V | Q | R | V | D | A | A | F | N | W | S | K | N |
| SEQ ID NO: 11 | G | F | P | P | T | V | R | K | I | D | A | A | I | S | D | K | E | K |
| SEQ ID NO: 12 | G | L | P | A | T | V | K | K | I | D | A | A | I | S | N | K | E | K |
| SEQ ID NO: 13 | G | F | P | S | T | I | R | K | I | D | A | A | I | S | D | K | E | R |
| SEQ ID NO: 14 | G | L | P | E | T | V | Q | K | I | D | A | A | I | S | L | K | D | Q |
| SEQ ID NO: 15 | G | F | P | S | S | V | Q | A | I | D | A | A | V | F | Y | R | — | — |
| SEQ ID NO: 16 | G | F | P | R | S | V | Q | A | I | D | A | A | V | S | Y | N | — | — |
| SEQ ID NO: 17 | G | F | P | R | S | V | Q | A | I | D | A | A | V | S | Y | N | — | — |
| SEQ ID NO: 18 | G | L | G | P | E | V | A | Q | V | T | G | A | L | P | R | P | E | — |
| SEQ ID NO: 19 | G | L | G | P | E | V | T | Q | V | T | G | A | L | P | Q | G | G | — |
| SEQ ID NO: 20 | G | L | G | A | D | V | A | Q | V | T | G | A | L | R | S | G | R | — |
| SEQ ID NO: 21 | G | L | G | P | E | V | T | H | V | S | G | L | L | P | R | R | P | — |
| SEQ ID NO: 22 | G | F | P | P | T | I | R | K | I | D | A | A | V | S | D | K | E | K |
| SEQ ID NO: 23 | G | F | P | P | T | V | K | K | I | D | A | A | V | F | E | K | E | K |
| SEQ ID NO: 24 | G | L | V | R | F | P | V | H | A | A | L | V | W | G | P | E | K | — |
| SEQ ID NO: 25 | G | L | Q | G | S | P | V | H | A | A | L | V | W | G | P | E | K | — |
| SEQ ID NO: 26 | G | F | P | N | F | V | K | K | I | D | A | A | V | F | N | P | R | F |
| SEQ ID NO: 27 | G | F | S | A | S | V | K | K | V | D | A | A | V | F | D | P | L | R |
| SEQ ID NO: 28 | G | F | P | D | F | V | K | K | I | D | A | A | V | F | N | P | S | L |
| SEQ ID NO: 29 | G | F | P | A | S | V | K | K | I | D | A | A | V | F | D | P | L | R |
| SEQ ID NO: 30 | G | F | P | K | H | V | K | K | I | S | A | A | L | H | F | E | D | S |
| SEQ ID NO: 31 | G | F | P | K | D | V | K | K | I | S | A | A | V | H | F | E | D | T |
| SEQ ID NO: 32 | G | L | P | K | E | V | K | K | I | S | A | A | V | H | F | E | D | T |
| SEQ ID NO: 33 | G | F | P | R | E | V | K | K | I | S | A | A | V | H | F | E | D | T |
| SEQ ID NO: 34 | R | G | L | P | T | D | K | — | I | D | A | A | L | F | W | M | P | N |
| SEQ ID NO: 35 | R | G | L | P | T | D | K | — | I | D | A | A | L | F | W | M | P | N |
| SEQ ID NO: 36 | R | R | L | P | T | D | K | — | I | D | A | A | L | F | W | M | P | N |
| SEQ ID NO: 37 | R | G | L | P | T | D | K | — | I | D | A | A | L | F | W | M | P | N |
| SEQ ID NO: 38 | R | G | L | P | T | D | K | — | I | D | A | A | L | F | W | M | P | N |
| SEQ ID NO: 39 | L | G | I | P | Y | D | R | — | I | D | T | A | I | W | W | E | P | T |
| SEQ ID NO: 40 | T | D | I | P | Y | D | R | — | I | D | T | A | I | W | W | E | P | T |
| SEQ ID NO: 41 | S | G | I | P | P | H | G | — | I | D | S | A | I | W | W | E | D | V |
| SEQ ID NO: 42 | N | G | I | P | P | H | G | — | I | D | S | A | I | W | W | E | D | V |
| SEQ ID NO: 43 | N | G | I | P | P | H | G | — | I | D | S | A | I | W | W | E | D | V |
| SEQ ID NO: 44 | — | — | L | P | P | G | G | — | I | D | A | A | F | S | W | A | H | N |
| SEQ ID NO: 45 | — | — | L | P | P | G | G | — | I | D | A | V | F | S | W | A | H | N |
| SEQ ID NO: 46 | G | F | P | K | T | V | K | R | I | D | A | A | V | H | I | E | Q | L |
| SEQ ID NO: 47 | — | — | — | R | V | E | P | N | L | D | A | A | L | Y | W | P | L | N |
| SEQ ID NO: 48 | — | — | — | R | V | E | P | N | L | D | A | A | L | Y | W | P | V | N |
| SEQ ID NO: 49 | G | F | P | R | Y | V | Q | R | I | D | A | A | V | Y | L | K | D | A |
| SEQ ID NO: 50 | G | F | P | R | H | V | Q | Q | I | D | A | A | V | Y | L | R | E | P |
| SEQ ID NO: 51 | G | F | P | R | H | V | Q | R | I | D | A | A | V | Y | L | K | E | P |
| SEQ ID NO: 52 | G | F | P | R | Y | V | Q | R | I | D | A | A | V | H | L | K | D | T |
| SEQ ID NO: 53 | S | C | L | P | R | E | G | — | I | D | T | A | L | R | W | E | P | V |
| SEQ ID NO: 54 | S | C | L | P | R | E | G | — | I | D | T | A | L | R | W | E | P | V |
| SEQ ID NO: 55 | S | C | L | P | R | E | G | — | I | D | T | A | L | R | W | E | P | V |
| SEQ ID NO: 56 | — | — | L | P | P | G | E | E | V | D | A | V | F | S | W | P | Q | N |
| SEQ ID NO: 57 | — | A | G | G | L | P | R | H | P | D | A | A | L | F | F | P | P | L |
| SEQ ID NO: 58 | R | G | H | S | R | G | R | N | Q | — | — | — | — | N | S | R | R | P |
| SEQ ID NO: 59 | R | G | R | G | H | R | R | S | Q | S | — | — | S | N | S | R | R | S |
| SEQ ID NO: 60 | R | G | R | G | R | G | R | G | H | S | R | S | Q | K | S | H | R | Q |
| SEQ ID NO: 61 | Y | G | — | — | R | G | R | S | Q | — | — | — | — | N | S | R | R | L |

| | |
|---|---|
| 121 | 131 |

| | | | | | | | | | | | | | | | | | | | | |
|---|---|---|---|---|---|---|---|---|---|---|---|---|---|---|---|---|---|---|---|---|
| SEQ ID NO: 02 | K | K | T | Y | I | F | S | G | D | R | Y | W | K | Y | N | E | E | K | K | K |
| SEQ ID NO: 03 | R | E | L | L | W | H | G | F | A | E | F | L | I | F | L | L | P | L | I | N |
| SEQ ID NO: 04 | R | E | L | L | W | H | G | F | A | E | F | L | V | F | L | L | P | L | I | N |
| SEQ ID NO: 05 | K | K | T | Y | I | F | A | G | D | K | F | W | R | Y | N | E | V | K | K | K |
| SEQ ID NO: 06 | G | K | T | Y | F | F | V | A | N | K | C | W | R | Y | D | E | Y | K | Q | S |
| SEQ ID NO: 07 | G | K | T | Y | F | F | V | A | D | K | Y | W | R | Y | D | E | Y | K | R | S |
| SEQ ID NO: 08 | G | K | T | Y | F | F | V | A | N | K | Y | W | R | Y | D | E | Y | K | R | S |
| SEQ ID NO: 09 | G | K | T | Y | F | F | V | A | H | E | C | W | R | Y | D | E | Y | K | Q | S |
| SEQ ID NO: 10 | K | K | T | Y | I | F | A | G | D | K | F | W | R | Y | N | E | V | K | K | K |
| SEQ ID NO: 11 | N | K | T | Y | F | F | V | E | D | K | Y | W | R | F | D | E | K | R | N | S |
| SEQ ID NO: 12 | R | K | T | Y | F | F | V | E | D | K | Y | W | R | F | D | E | K | K | Q | S |
| SEQ ID NO: 13 | K | K | T | Y | F | F | V | E | D | K | Y | W | R | F | D | E | K | R | Q | S |
| SEQ ID NO: 14 | K | K | T | Y | F | F | V | E | D | K | F | W | R | F | D | E | K | K | Q | S |
| SEQ ID NO: 15 | S | K | T | Y | F | F | V | N | D | Q | F | W | R | Y | D | N | Q | R | Q | F |
| SEQ ID NO: 16 | G | K | T | Y | F | F | I | N | N | Q | C | W | R | Y | D | N | E | R | R | S |
| SEQ ID NO: 17 | G | K | T | Y | F | F | V | N | N | Q | C | W | R | Y | D | N | Q | R | R | S |
| SEQ ID NO: 18 | G | K | V | L | L | F | S | G | Q | S | F | W | R | F | D | V | K | T | Q | K |
| SEQ ID NO: 19 | G | K | V | L | L | F | S | R | Q | R | F | W | S | F | D | V | K | T | Q | T |
| SEQ ID NO: 20 | G | K | M | L | L | F | S | G | R | R | L | W | R | F | D | V | K | A | Q | M |
| SEQ ID NO: 21 | G | K | A | L | L | F | S | K | G | R | V | W | R | F | D | L | K | S | Q | K |
| SEQ ID NO: 22 | K | K | T | Y | F | F | A | A | D | K | Y | W | R | F | D | E | N | S | Q | S |
| SEQ ID NO: 23 | K | K | T | Y | F | F | V | G | D | K | V | W | R | F | D | E | T | R | H | V |
| SEQ ID NO: 24 | N | K | I | V | F | F | R | G | R | D | Y | W | R | F | H | P | S | T | R | R |
| SEQ ID NO: 25 | N | K | I | Y | F | F | R | G | G | D | Y | W | R | F | H | P | R | T | Q | R |
| SEQ ID NO: 26 | Y | R | T | Y | F | F | V | D | N | Q | Y | W | R | Y | D | E | R | R | Q | M |
| SEQ ID NO: 27 | Q | K | V | Y | F | F | V | D | K | H | Y | W | R | Y | D | V | R | Q | E | L |
| SEQ ID NO: 28 | R | K | T | Y | F | F | V | D | N | L | Y | W | R | Y | D | E | R | R | E | V |
| SEQ ID NO: 29 | Q | K | V | Y | F | F | V | D | K | Q | Y | W | R | Y | D | V | R | Q | E | L |
| SEQ ID NO: 30 | G | K | T | L | F | F | S | E | N | Q | V | W | S | Y | D | D | T | N | H | V |
| SEQ ID NO: 31 | G | K | T | L | F | F | S | G | N | Q | V | W | R | Y | D | D | T | N | R | M |
| SEQ ID NO: 32 | G | K | T | L | L | F | S | G | N | Q | V | W | R | Y | D | D | T | N | H | I |
| SEQ ID NO: 33 | G | K | T | L | F | F | S | G | N | Q | V | W | S | Y | D | D | T | N | H | T |
| SEQ ID NO: 34 | G | K | T | Y | F | F | R | G | N | K | Y | Y | R | F | N | E | E | L | R | A |
| SEQ ID NO: 35 | G | K | T | Y | F | F | R | G | N | K | Y | Y | R | F | N | E | E | F | R | A |
| SEQ ID NO: 36 | G | K | D | Y | F | F | R | G | N | K | Y | Y | R | F | N | E | E | L | R | A |
| SEQ ID NO: 37 | G | K | T | Y | F | F | R | G | N | K | Y | Y | R | F | N | E | E | L | R | A |
| SEQ ID NO: 38 | G | K | T | Y | F | F | R | G | N | K | Y | Y | R | F | N | E | E | F | R | A |
| SEQ ID NO: 39 | G | H | T | F | F | F | Q | E | D | R | Y | W | R | F | N | E | E | T | Q | R |
| SEQ ID NO: 40 | G | H | T | F | F | F | Q | A | D | R | Y | W | R | F | N | E | E | T | Q | H |
| SEQ ID NO: 41 | G | K | T | Y | F | F | K | G | D | R | Y | W | R | Y | S | E | E | M | K | T |
| SEQ ID NO: 42 | G | K | T | Y | F | F | K | G | D | R | Y | W | R | Y | S | E | E | M | K | T |
| SEQ ID NO: 43 | G | K | T | Y | F | F | K | G | D | R | Y | W | R | Y | S | E | E | M | K | T |
| SEQ ID NO: 44 | D | R | T | Y | F | F | K | D | Q | L | Y | W | R | Y | D | D | H | T | R | H |
| SEQ ID NO: 45 | D | R | T | Y | F | F | K | D | Q | L | Y | W | R | Y | D | D | H | T | R | R |
| SEQ ID NO: 46 | G | K | T | Y | F | F | A | A | K | K | Y | W | S | Y | D | E | D | K | K | Q |
| SEQ ID NO: 47 | Q | K | V | F | L | F | K | G | S | G | Y | W | Q | W | D | E | L | A | R | T |
| SEQ ID NO: 48 | Q | K | V | F | L | F | K | G | S | G | Y | W | Q | W | D | E | L | A | R | T |
| SEQ ID NO: 49 | Q | K | T | L | F | F | V | G | D | E | Y | Y | S | Y | D | E | R | K | R | K |
| SEQ ID NO: 50 | Q | K | T | L | F | F | V | G | D | E | Y | Y | S | Y | D | E | R | K | R | K |
| SEQ ID NO: 51 | Q | K | T | L | F | F | V | G | E | E | Y | Y | S | Y | D | E | R | K | K | K |
| SEQ ID NO: 52 | Q | K | T | L | F | F | V | G | D | E | Y | Y | S | Y | D | E | R | K | R | K |
| SEQ ID NO: 53 | G | K | T | Y | F | F | K | G | E | R | Y | W | R | Y | S | E | E | R | R | A |
| SEQ ID NO: 54 | G | K | T | Y | F | F | K | G | E | R | Y | W | R | Y | S | E | E | R | R | A |
| SEQ ID NO: 55 | G | K | T | Y | F | F | K | G | E | R | Y | W | R | Y | S | E | E | R | R | A |
| SEQ ID NO: 56 | G | K | T | Y | L | V | R | G | R | Q | Y | W | R | Y | D | E | A | A | A | R |
| SEQ ID NO: 57 | R | R | L | I | L | F | K | G | A | R | Y | Y | V | L | A | R | G | G | L | Q |
| SEQ ID NO: 58 | S | R | A | T | W | L | S | — | — | — | L | F | S | S | E | E | S | N | L | G |
| SEQ ID NO: 59 | S | R | S | I | W | F | S | — | — | — | L | F | S | S | E | E | S | G | L | G |
| SEQ ID NO: 60 | S | R | S | T | W | L | P | — | — | — | W | F | S | S | E | E | T | G | P | G |
| SEQ ID NO: 61 | S | R | S | I | S | R | L | — | — | — | W | F | S | S | E | E | V | S | L | G |

| | |
|---|---|
| 141 | 151 |

| | | | | | | | | | | | | | | | | | | | | |
|---|---|---|---|---|---|---|---|---|---|---|---|---|---|---|---|---|---|---|---|---|
| SEQ ID NO: 02 | M | E | L | A | T | P | K | F | I | A | D | S | W | N | G | V | P | D | N | L |
| SEQ ID NO: 03 | I | Q | K | L | K | A | K | L | S | S | W | C | T | L | C | T | G | A | A | G |
| SEQ ID NO: 04 | I | Q | K | L | K | A | K | L | S | S | W | C | I | P | L | T | S | T | A | G |
| SEQ ID NO: 05 | M | D | P | G | F | P | R | L | I | A | D | A | W | N | A | I | P | D | H | L |
| SEQ ID NO: 06 | M | D | A | G | Y | P | K | M | I | A | E | D | F | P | G | I | G | N | K | V |
| SEQ ID NO: 07 | M | D | A | G | Y | P | K | M | I | A | D | D | F | P | G | I | G | D | K | V |
| SEQ ID NO: 08 | M | D | P | G | Y | P | K | M | I | A | H | D | F | P | G | I | G | H | K | V |
| SEQ ID NO: 09 | M | D | T | G | Y | P | K | M | I | A | E | E | F | P | G | I | G | N | K | V |
| SEQ ID NO: 10 | M | D | P | G | F | P | K | L | I | A | D | A | W | N | A | I | P | D | N | L |
| SEQ ID NO: 11 | M | E | P | G | F | P | K | Q | I | A | E | D | F | P | G | I | D | S | K | I |
| SEQ ID NO: 12 | M | E | P | G | F | P | R | K | I | A | E | D | F | P | G | V | D | S | R | V |
| SEQ ID NO: 13 | L | E | P | G | F | P | R | H | I | A | E | D | F | P | G | I | N | P | K | I |
| SEQ ID NO: 14 | M | D | P | E | F | P | R | K | I | A | E | N | F | P | G | I | G | T | K | V |
| SEQ ID NO: 15 | M | E | P | G | Y | P | K | S | I | S | G | A | F | P | G | I | E | S | K | V |
| SEQ ID NO: 16 | M | D | P | G | Y | P | K | S | I | P | S | M | F | P | G | V | N | C | R | V |
| SEQ ID NO: 17 | M | D | P | G | Y | P | T | S | I | A | S | V | F | P | G | I | N | C | R | I |
| SEQ ID NO: 18 | V | D | P | Q | S | V | T | P | V | D | Q | M | F | P | G | V | P | I | S | T |
| SEQ ID NO: 19 | V | D | P | R | S | A | G | S | V | E | Q | M | Y | P | G | V | P | L | N | T |
| SEQ ID NO: 20 | V | D | P | R | S | A | S | E | V | D | R | M | F | P | G | V | P | L | D | T |
| SEQ ID NO: 21 | V | D | P | Q | S | V | I | R | V | D | K | E | F | S | G | V | P | W | N | S |
| SEQ ID NO: 22 | M | E | Q | G | F | P | R | L | I | A | D | D | F | P | G | V | E | P | K | V |
| SEQ ID NO: 23 | M | D | K | G | F | P | R | Q | I | T | D | D | F | P | G | I | E | P | Q | V |
| SEQ ID NO: 24 | V | D | S | — | P | V | P | R | R | A | T | D | W | R | G | V | P | S | E | I |
| SEQ ID NO: 25 | V | D | N | — | P | V | P | R | R | S | T | D | W | R | G | V | P | S | E | I |
| SEQ ID NO: 26 | M | D | P | G | Y | P | K | L | I | T | K | N | F | Q | G | I | G | P | K | I |
| SEQ ID NO: 27 | M | D | P | A | Y | P | K | L | I | S | T | H | F | P | G | I | K | P | K | I |
| SEQ ID NO: 28 | M | D | A | G | Y | P | K | L | I | T | K | H | F | P | G | I | G | P | K | I |
| SEQ ID NO: 29 | M | D | A | A | Y | P | K | L | I | S | T | H | F | P | G | I | R | P | K | I |
| SEQ ID NO: 30 | M | D | K | D | Y | P | R | L | I | E | E | V | F | P | G | I | G | D | K | V |
| SEQ ID NO: 31 | M | D | K | D | Y | P | R | L | I | E | E | D | F | P | G | I | G | D | K | V |
| SEQ ID NO: 32 | M | D | K | D | Y | P | R | L | I | E | E | D | F | P | G | I | G | D | K | V |
| SEQ ID NO: 33 | M | D | Q | D | Y | P | R | L | I | E | E | E | F | P | G | I | G | G | K | V |
| SEQ ID NO: 34 | V | D | S | E | Y | P | K | N | I | K | — | V | W | E | G | I | P | E | S | P |
| SEQ ID NO: 35 | V | D | S | E | Y | P | K | N | I | K | — | V | W | E | G | I | P | E | S | P |
| SEQ ID NO: 36 | V | D | S | E | Y | P | K | N | I | K | — | V | W | E | G | I | P | E | S | P |
| SEQ ID NO: 37 | V | D | S | E | Y | P | K | N | I | K | — | V | W | E | G | I | P | E | S | P |
| SEQ ID NO: 38 | V | D | S | E | Y | P | K | N | I | K | — | V | W | E | G | I | P | E | S | P |
| SEQ ID NO: 39 | G | D | P | G | Y | P | K | P | I | S | — | V | W | Q | G | I | P | A | S | P |
| SEQ ID NO: 40 | G | D | P | G | Y | P | K | P | I | S | — | V | W | Q | G | I | P | T | S | P |
| SEQ ID NO: 41 | M | D | P | G | Y | P | K | P | I | T | — | V | W | K | G | I | P | E | S | P |
| SEQ ID NO: 42 | M | D | P | G | Y | P | K | P | I | T | — | I | W | K | G | I | P | E | S | P |
| SEQ ID NO: 43 | M | D | P | G | Y | P | K | P | I | T | — | I | W | K | G | I | P | E | S | P |
| SEQ ID NO: 44 | M | D | P | G | Y | P | A | Q | S | P | — | L | W | R | G | V | P | S | T | L |
| SEQ ID NO: 45 | M | D | P | G | Y | P | A | Q | G | P | — | L | W | R | G | V | P | S | M | L |
| SEQ ID NO: 46 | M | D | K | G | F | P | K | Q | I | S | N | D | F | P | G | I | P | D | K | I |
| SEQ ID NO: 47 | D | F | S | S | Y | P | K | P | I | K | G | L | F | T | G | V | P | N | Q | P |
| SEQ ID NO: 48 | D | L | S | R | Y | P | K | P | I | K | E | L | F | T | G | V | P | D | R | P |
| SEQ ID NO: 49 | M | E | K | D | Y | P | K | S | T | E | E | E | F | S | G | V | N | G | Q | I |
| SEQ ID NO: 50 | M | E | K | D | Y | P | K | N | T | E | E | E | F | S | G | V | N | G | Q | I |
| SEQ ID NO: 51 | M | E | K | D | Y | P | K | N | T | E | E | E | F | S | G | V | S | G | H | I |
| SEQ ID NO: 52 | M | D | K | D | Y | P | K | N | T | E | E | E | F | S | G | V | N | G | Q | I |
| SEQ ID NO: 53 | T | D | P | G | Y | P | K | P | I | T | — | V | W | K | G | I | P | Q | A | P |
| SEQ ID NO: 54 | T | D | P | G | Y | P | K | P | I | T | — | V | W | K | G | I | P | Q | A | P |
| SEQ ID NO: 55 | T | D | P | G | Y | P | K | P | I | T | — | V | W | K | G | I | P | Q | A | P |
| SEQ ID NO: 56 | P | D | P | G | Y | P | R | D | L | S | — | L | W | E | G | A | P | P | S | P |
| SEQ ID NO: 57 | V | E | P | Y | Y | P | R | S | L | Q | — | D | W | G | G | I | P | E | E | V |
| SEQ ID NO: 58 | A | N | N | Y | D | D | Y | R | M | D | W | L | V | P | A | T | C | E | P | I |
| SEQ ID NO: 59 | T | Y | N | N | Y | D | Y | D | M | D | W | L | V | P | A | T | C | E | P | I |
| SEQ ID NO: 60 | G | Y | N | Y | D | D | Y | K | M | D | W | L | V | P | A | T | C | E | P | I |
| SEQ ID NO: 61 | P | Y | N | Y | E | D | Y | E | T | S | W | L | K | P | A | T | S | E | P | I |

| | |
|---|---|
| 161 | 171 |

| | | | | | | | | | | | | | | | | | | | | |
|---|---|---|---|---|---|---|---|---|---|---|---|---|---|---|---|---|---|---|---|---|
| SEQ ID NO: 02 | D | A | V | L | G | L | T | D | S | G | Y | T | Y | F | F | K | D | Q | Y | Y |
| SEQ ID NO: 03 | H | A | S | T | L | G | S | S | G | K | E | C | A | L | C | G | E | W | P | T |
| SEQ ID NO: 04 | S | D | S | T | L | G | S | S | G | K | E | C | A | L | C | G | E | W | P | T |
| SEQ ID NO: 05 | D | A | V | V | D | L | Q | G | S | G | H | S | Y | F | F | K | G | T | Y | Y |
| SEQ ID NO: 06 | D | A | V | F | Q | K | G | — | — | G | F | F | Y | F | F | H | G | R | R | Q |
| SEQ ID NO: 07 | D | A | V | F | Q | K | D | — | — | G | F | F | Y | F | F | H | G | T | R | Q |
| SEQ ID NO: 08 | D | A | V | F | M | K | D | — | — | G | F | F | Y | F | F | H | G | T | R | Q |
| SEQ ID NO: 09 | D | A | V | F | Q | K | D | — | — | G | F | L | Y | F | F | H | G | T | R | Q |
| SEQ ID NO: 10 | D | A | V | V | D | L | Q | G | G | G | H | S | Y | F | F | K | G | A | Y | Y |
| SEQ ID NO: 11 | D | A | V | F | E | E | F | — | — | G | F | F | Y | F | F | T | G | S | S | Q |
| SEQ ID NO: 12 | D | A | V | F | E | A | F | — | — | G | F | L | Y | F | F | S | G | S | S | Q |
| SEQ ID NO: 13 | D | A | V | F | E | A | F | — | — | G | F | F | Y | F | F | S | G | S | S | Q |
| SEQ ID NO: 14 | D | A | V | F | E | A | F | — | — | G | F | L | Y | F | F | S | G | S | S | Q |
| SEQ ID NO: 15 | D | A | V | F | Q | Q | E | — | — | H | F | F | H | V | F | S | G | P | R | Y |
| SEQ ID NO: 16 | D | A | V | F | L | Q | D | — | — | S | F | F | L | F | F | S | G | P | Q | Y |
| SEQ ID NO: 17 | D | A | V | F | Q | Q | D | — | — | S | F | F | L | F | F | S | G | P | Q | Y |
| SEQ ID NO: 18 | H | D | I | F | Q | Y | G | E | — | — | K | A | Y | F | C | Q | D | H | F | Y |
| SEQ ID NO: 19 | H | D | I | F | Q | Y | G | E | — | — | K | A | Y | F | C | Q | D | R | F | Y |
| SEQ ID NO: 20 | H | D | V | F | Q | Y | R | E | — | — | K | A | Y | F | C | Q | D | R | F | Y |
| SEQ ID NO: 21 | H | D | I | F | Q | Y | Q | D | — | — | K | A | Y | F | C | H | G | K | F | F |
| SEQ ID NO: 22 | D | A | V | L | Q | A | F | — | — | G | F | F | Y | F | F | S | G | S | S | Q |
| SEQ ID NO: 23 | D | A | V | L | H | E | F | — | — | G | F | F | Y | F | F | R | G | S | S | Q |
| SEQ ID NO: 24 | D | A | A | F | Q | D | A | D | G | — | Y | A | Y | F | L | R | G | R | L | Y |
| SEQ ID NO: 25 | D | A | A | F | Q | D | A | E | G | — | Y | A | Y | F | L | R | G | H | L | Y |
| SEQ ID NO: 26 | D | A | V | F | Y | S | K | N | — | K | Y | Y | Y | F | F | Q | G | S | N | Q |
| SEQ ID NO: 27 | D | A | V | L | Y | F | K | — | — | R | H | Y | Y | I | F | Q | G | A | Y | Q |
| SEQ ID NO: 28 | D | A | V | F | Y | F | Q | — | — | R | Y | Y | Y | F | F | Q | G | P | N | Q |
| SEQ ID NO: 29 | D | A | V | L | Y | F | K | — | — | R | H | Y | Y | I | F | Q | G | A | Y | Q |
| SEQ ID NO: 30 | D | A | V | Y | Q | K | N | — | — | G | Y | I | Y | F | F | N | G | P | I | Q |
| SEQ ID NO: 31 | D | A | V | Y | E | K | N | — | — | G | Y | I | Y | F | F | N | G | P | I | Q |
| SEQ ID NO: 32 | D | A | V | Y | E | K | N | — | — | G | Y | I | Y | F | F | N | G | P | I | Q |
| SEQ ID NO: 33 | D | A | V | Y | E | K | N | — | — | G | Y | I | Y | F | F | N | G | P | I | Q |
| SEQ ID NO: 34 | R | G | S | F | M | G | S | D | E | V | F | T | Y | F | Y | K | G | N | K | Y |
| SEQ ID NO: 35 | R | G | S | F | M | G | S | D | E | V | F | T | Y | F | Y | K | G | N | K | Y |
| SEQ ID NO: 36 | R | G | S | F | M | G | S | D | E | V | F | T | Y | F | Y | K | G | N | K | Y |
| SEQ ID NO: 37 | R | G | S | F | M | G | S | D | E | V | F | T | Y | F | Y | K | G | N | K | Y |
| SEQ ID NO: 38 | R | G | S | F | M | G | S | D | E | V | F | T | Y | F | Y | K | G | N | K | Y |
| SEQ ID NO: 39 | K | G | A | F | L | S | N | D | A | A | Y | T | Y | F | Y | K | G | T | K | Y |
| SEQ ID NO: 40 | K | G | A | F | L | S | N | D | A | A | Y | T | Y | F | Y | K | G | T | K | Y |
| SEQ ID NO: 41 | Q | G | A | F | V | H | K | E | N | G | F | T | Y | F | Y | K | G | K | E | Y |
| SEQ ID NO: 42 | Q | G | A | F | V | H | K | E | N | G | F | T | Y | F | Y | K | G | K | E | Y |
| SEQ ID NO: 43 | Q | G | A | F | V | H | K | E | N | G | F | T | Y | F | Y | K | G | K | E | Y |
| SEQ ID NO: 44 | D | D | A | M | R | W | S | D | G | — | A | S | Y | F | F | R | G | Q | E | Y |
| SEQ ID NO: 45 | D | D | A | M | R | W | S | D | G | — | A | S | Y | F | F | R | G | Q | E | Y |
| SEQ ID NO: 46 | D | A | A | F | Y | Y | R | — | — | G | R | L | Y | F | F | I | G | R | S | Q |
| SEQ ID NO: 47 | S | A | A | M | S | W | Q | D | — | G | R | V | Y | F | F | K | G | K | V | Y |
| SEQ ID NO: 48 | S | A | A | M | S | W | Q | D | — | G | Q | V | Y | F | F | K | G | K | E | Y |
| SEQ ID NO: 49 | D | A | A | V | E | L | N | — | — | G | V | I | Y | F | F | S | G | P | K | A |
| SEQ ID NO: 50 | D | A | A | V | E | L | N | — | — | G | Y | I | Y | F | F | S | G | P | K | T |
| SEQ ID NO: 51 | D | A | A | V | E | L | N | — | — | G | V | I | Y | F | F | S | G | R | K | T |
| SEQ ID NO: 52 | D | A | A | V | E | L | N | — | — | G | Y | I | Y | F | F | S | G | P | K | A |
| SEQ ID NO: 53 | Q | G | A | F | I | S | K | E | G | Y | Y | T | Y | F | Y | K | G | R | D | Y |
| SEQ ID NO: 54 | Q | G | A | F | I | S | K | E | G | Y | Y | T | Y | F | Y | K | G | R | D | Y |
| SEQ ID NO: 55 | Q | G | A | F | I | S | K | E | G | Y | Y | T | Y | F | Y | K | G | R | D | Y |
| SEQ ID NO: 56 | D | D | V | T | V | S | N | A | G | — | D | T | Y | F | F | K | G | A | H | Y |
| SEQ ID NO: 57 | S | G | A | L | P | R | P | D | — | G | S | I | I | F | F | R | D | D | R | Y |
| SEQ ID NO: 58 | Q | S | — | — | — | — | — | — | — | — | — | V | F | F | F | S | G | D | K | Y |
| SEQ ID NO: 59 | Q | S | — | — | — | — | — | — | — | — | — | V | Y | F | F | S | G | D | K | Y |
| SEQ ID NO: 60 | Q | S | — | — | — | — | — | — | — | — | — | V | Y | F | F | S | G | E | E | Y |
| SEQ ID NO: 61 | Q | S | — | — | — | — | — | — | — | — | — | V | Y | F | F | S | G | D | K | Y |

| | |
|---|---|
| 181 | 191 |

| | | | | | | | | | | | | | | | | | | | | |
|---|---|---|---|---|---|---|---|---|---|---|---|---|---|---|---|---|---|---|---|---|
| SEQ ID NO: 02 | L | Q | M | E | D | K | S | L | K | I | — | — | — | — | — | — | — | V | K | I |
| SEQ ID NO: 03 | M | P | H | T | I | G | C | E | H | V | F | C | Y | — | — | — | — | Y | C | V |
| SEQ ID NO: 04 | M | P | H | T | I | G | C | E | H | V | F | C | Y | — | — | — | — | Y | C | V |
| SEQ ID NO: 05 | L | K | L | E | N | Q | S | L | K | S | — | — | — | — | — | — | — | V | K | V |
| SEQ ID NO: 06 | Y | K | F | D | P | Q | T | K | R | I | — | — | — | — | — | — | — | L | T | L |
| SEQ ID NO: 07 | Y | K | F | D | P | K | T | K | R | I | — | — | — | — | — | — | — | L | T | L |
| SEQ ID NO: 08 | Y | K | F | D | P | K | T | K | R | I | — | — | — | — | — | — | — | L | T | L |
| SEQ ID NO: 09 | Y | Q | F | D | F | K | T | K | R | I | — | — | — | — | — | — | — | L | T | L |
| SEQ ID NO: 10 | L | K | L | E | N | Q | S | L | K | S | — | — | — | — | — | — | — | V | K | F |
| SEQ ID NO: 11 | L | E | F | D | P | N | A | K | K | V | — | — | — | — | — | — | — | T | H | T |
| SEQ ID NO: 12 | L | E | F | D | P | N | A | K | K | V | — | — | — | — | — | — | — | T | H | I |
| SEQ ID NO: 13 | S | E | F | D | P | N | A | K | K | V | — | — | — | — | — | — | — | T | H | V |
| SEQ ID NO: 14 | L | E | F | D | P | N | A | G | K | V | — | — | — | — | — | — | — | T | H | I |
| SEQ ID NO: 15 | Y | A | F | D | L | I | A | Q | R | V | — | — | — | — | — | — | — | T | R | V |
| SEQ ID NO: 16 | F | A | F | N | F | V | S | H | R | V | — | — | — | — | — | — | — | T | R | V |
| SEQ ID NO: 17 | F | A | F | N | L | V | S | R | R | V | — | — | — | — | — | — | — | T | R | V |
| SEQ ID NO: 18 | W | R | V | S | S | Q | N | E | V | N | — | — | — | — | — | — | — | Q | V | D |
| SEQ ID NO: 19 | W | R | V | N | S | R | N | E | V | N | — | — | — | — | — | — | — | Q | V | D |
| SEQ ID NO: 20 | W | R | V | S | S | R | S | E | L | N | — | — | — | — | — | — | — | Q | V | D |
| SEQ ID NO: 21 | W | R | V | S | F | Q | N | E | V | N | K | V | D | P | E | V | N | Q | V | D |
| SEQ ID NO: 22 | F | E | F | D | P | N | A | R | M | V | — | — | — | — | — | — | — | T | H | I |
| SEQ ID NO: 23 | F | E | F | D | P | N | A | R | T | V | — | — | — | — | — | — | — | T | H | I |
| SEQ ID NO: 24 | W | K | F | D | P | V | K | V | K | A | — | — | — | — | — | — | — | L | E | G |
| SEQ ID NO: 25 | W | K | F | D | P | V | K | V | K | V | — | — | — | — | — | — | — | L | E | G |
| SEQ ID NO: 26 | F | E | Y | D | F | L | L | Q | R | I | — | — | — | — | — | — | — | T | K | T |
| SEQ ID NO: 27 | L | E | Y | D | P | L | F | R | R | V | — | — | — | — | — | — | — | T | K | T |
| SEQ ID NO: 28 | L | E | Y | D | T | F | S | S | R | V | — | — | — | — | — | — | — | T | K | K |
| SEQ ID NO: 29 | L | E | Y | D | P | L | L | D | R | V | — | — | — | — | — | — | — | T | K | T |
| SEQ ID NO: 30 | F | E | Y | S | I | W | S | N | R | I | — | — | — | — | — | — | — | V | R | V |
| SEQ ID NO: 31 | F | E | Y | S | I | W | S | N | R | I | — | — | — | — | — | — | — | V | R | V |
| SEQ ID NO: 32 | F | E | Y | S | I | W | S | N | R | I | — | — | — | — | — | — | — | V | R | V |
| SEQ ID NO: 33 | F | E | Y | S | I | W | S | K | R | I | — | — | — | — | — | — | — | V | R | V |
| SEQ ID NO: 34 | W | K | F | N | N | Q | K | L | K | V | — | — | — | — | — | — | — | E | P | G |
| SEQ ID NO: 35 | W | K | F | N | N | Q | K | L | K | V | — | — | — | — | — | — | — | E | P | G |
| SEQ ID NO: 36 | W | K | F | N | N | Q | K | L | K | V | — | — | — | — | — | — | — | E | P | G |
| SEQ ID NO: 37 | W | K | F | N | N | Q | K | L | K | V | — | — | — | — | — | — | — | E | P | G |
| SEQ ID NO: 38 | W | K | F | N | N | Q | K | L | K | V | — | — | — | — | — | — | — | E | P | G |
| SEQ ID NO: 39 | W | K | F | D | N | E | R | L | R | M | — | — | — | — | — | — | — | E | P | G |
| SEQ ID NO: 40 | W | K | F | N | N | E | R | L | R | M | — | — | — | — | — | — | — | E | P | G |
| SEQ ID NO: 41 | W | K | F | N | N | Q | I | L | K | V | — | — | — | — | — | — | — | E | P | G |
| SEQ ID NO: 42 | W | K | F | N | N | Q | I | L | K | V | — | — | — | — | — | — | — | E | P | G |
| SEQ ID NO: 43 | W | K | F | N | N | Q | I | L | K | V | — | — | — | — | — | — | — | E | P | G |
| SEQ ID NO: 44 | W | K | V | L | D | G | E | L | E | V | — | — | — | — | — | — | — | A | P | G |
| SEQ ID NO: 45 | W | K | V | L | D | G | E | L | E | A | — | — | — | — | — | — | — | A | P | G |
| SEQ ID NO: 46 | F | E | Y | N | I | N | S | K | R | I | — | — | — | — | — | — | — | V | Q | V |
| SEQ ID NO: 47 | W | R | L | N | Q | Q | L | R | V | E | — | — | — | — | — | — | — | — | K | G |
| SEQ ID NO: 48 | W | R | L | N | Q | Q | L | R | V | A | — | — | — | — | — | — | — | — | K | G |
| SEQ ID NO: 49 | Y | K | S | D | T | E | K | E | D | V | — | — | — | — | — | — | — | V | S | E |
| SEQ ID NO: 50 | Y | K | Y | D | T | E | K | E | D | V | — | — | — | — | — | — | — | V | S | V |
| SEQ ID NO: 51 | F | K | Y | D | T | E | K | E | D | V | — | — | — | — | — | — | — | V | S | V |
| SEQ ID NO: 52 | Y | K | Y | D | T | E | K | E | D | V | — | — | — | — | — | — | — | V | S | V |
| SEQ ID NO: 53 | W | K | F | D | N | Q | K | L | S | V | — | — | — | — | — | — | — | E | P | G |
| SEQ ID NO: 54 | W | K | F | D | N | Q | K | L | S | V | — | — | — | — | — | — | — | E | P | G |
| SEQ ID NO: 55 | W | K | F | D | N | Q | K | L | S | V | — | — | — | — | — | — | — | E | P | G |
| SEQ ID NO: 56 | W | R | F | P | K | N | S | I | K | T | — | — | — | — | — | — | — | E | P | D |
| SEQ ID NO: 57 | W | R | L | D | Q | A | K | L | Q | A | — | — | — | — | — | — | — | T | T | S |
| SEQ ID NO: 58 | Y | R | V | N | L | R | T | R | R | V | D | T | — | — | — | — | V | D | P | P |
| SEQ ID NO: 59 | Y | R | V | N | L | R | T | R | R | V | D | S | — | — | — | — | V | N | P | P |
| SEQ ID NO: 60 | Y | R | V | N | L | R | T | Q | R | V | D | T | — | — | — | — | V | T | P | P |
| SEQ ID NO: 61 | Y | R | V | N | L | R | T | Q | R | V | D | T | — | — | — | — | V | N | P | P |

| | |
|---|---|
| 201 | 210 |

| | | | | | | | | | | |
|---|---|---|---|---|---|---|---|---|---|---|
| SEQ ID NO: 02 | G | K | I | S | S | D | W | L | G | C |
| SEQ ID NO: 03 | K | S | S | F | L | F | Y | F | T | C |
| SEQ ID NO: 04 | K | S | S | F | L | F | Y | F | T | C |
| SEQ ID NO: 05 | G | S | I | K | T | D | W | L | G | C |
| SEQ ID NO: 06 | L | K | A | N | S | — | W | F | N | C |
| SEQ ID NO: 07 | Q | K | A | N | S | — | W | F | N | C |
| SEQ ID NO: 08 | Q | K | A | N | S | — | W | F | N | C |
| SEQ ID NO: 09 | Q | K | A | N | S | — | W | F | N | C |
| SEQ ID NO: 10 | G | S | I | K | S | D | W | L | G | C |
| SEQ ID NO: 11 | L | K | S | N | S | — | W | L | N | C |
| SEQ ID NO: 12 | L | K | S | N | S | — | W | F | N | C |
| SEQ ID NO: 13 | L | K | S | N | S | — | W | F | Q | C |
| SEQ ID NO: 14 | L | K | S | N | S | — | W | F | N | C |
| SEQ ID NO: 15 | A | R | G | N | K | — | W | L | N | C |
| SEQ ID NO: 16 | A | R | S | N | L | — | W | L | N | C |
| SEQ ID NO: 17 | A | R | S | N | L | — | W | L | N | C |
| SEQ ID NO: 18 | Y | V | G | Y | V | T | L | L | K | C |
| SEQ ID NO: 19 | E | V | G | Y | V | T | I | L | Q | C |
| SEQ ID NO: 20 | Q | V | G | Y | V | T | I | L | Q | C |
| SEQ ID NO: 21 | D | V | G | Y | V | T | L | L | Q | C |
| SEQ ID NO: 22 | L | K | S | N | S | — | W | L | H | C |
| SEQ ID NO: 23 | L | K | S | N | S | — | W | L | L | C |
| SEQ ID NO: 24 | F | P | R | L | V | G | F | F | G | C |
| SEQ ID NO: 25 | F | P | R | P | V | G | F | F | D | C |
| SEQ ID NO: 26 | L | K | S | N | S | — | W | F | G | C |
| SEQ ID NO: 27 | L | K | S | T | S | — | W | F | G | C |
| SEQ ID NO: 28 | L | K | S | N | S | — | W | F | D | C |
| SEQ ID NO: 29 | L | S | S | T | S | — | W | F | G | C |
| SEQ ID NO: 30 | M | T | T | N | S | — | L | L | W | C |
| SEQ ID NO: 31 | M | P | T | N | S | — | L | L | W | C |
| SEQ ID NO: 32 | M | P | A | N | S | — | I | L | W | C |
| SEQ ID NO: 33 | M | P | T | N | S | — | L | L | W | C |
| SEQ ID NO: 34 | Y | P | K | S | A | L | W | M | G | C |
| SEQ ID NO: 35 | Y | P | K | S | A | L | W | M | G | C |
| SEQ ID NO: 36 | Y | P | K | S | A | L | W | M | G | C |
| SEQ ID NO: 37 | Y | P | K | S | A | L | W | M | G | C |
| SEQ ID NO: 38 | Y | P | K | S | A | L | W | M | G | C |
| SEQ ID NO: 39 | Y | P | K | S | I | L | F | M | G | C |
| SEQ ID NO: 40 | H | P | K | S | I | L | F | M | G | C |
| SEQ ID NO: 41 | Y | P | R | S | I | L | F | M | G | C |
| SEQ ID NO: 42 | Y | P | R | S | I | L | F | M | G | C |
| SEQ ID NO: 43 | Y | P | R | S | I | L | F | M | G | C |
| SEQ ID NO: 44 | Y | P | Q | S | T | A | W | L | V | C |
| SEQ ID NO: 45 | Y | P | Q | S | T | A | W | L | V | C |
| SEQ ID NO: 46 | L | R | S | N | S | — | W | L | G | C |
| SEQ ID NO: 47 | Y | P | R | N | I | S | W | M | H | C |
| SEQ ID NO: 48 | Y | P | R | N | T | T | W | M | H | C |
| SEQ ID NO: 49 | L | K | S | S | S | — | W | I | G | C |
| SEQ ID NO: 50 | V | K | S | S | S | — | W | I | G | C |
| SEQ ID NO: 51 | V | K | S | S | S | — | W | I | G | C |
| SEQ ID NO: 52 | L | K | S | N | S | — | W | I | G | C |
| SEQ ID NO: 53 | Y | P | R | N | I | L | W | M | G | C |
| SEQ ID NO: 54 | Y | P | R | N | I | L | W | M | G | C |
| SEQ ID NO: 55 | Y | P | R | N | I | L | W | M | G | C |
| SEQ ID NO: 56 | A | P | Q | P | M | G | W | L | D | C |
| SEQ ID NO: 57 | G | R | W | A | T | E | W | M | G | C |
| SEQ ID NO: 58 | Y | P | R | S | I | A | W | L | G | C |
| SEQ ID NO: 59 | Y | P | R | S | I | A | W | L | G | C |
| SEQ ID NO: 60 | Y | P | R | S | I | A | W | L | G | C |
| SEQ ID NO: 61 | Y | P | R | S | I | A | W | L | G | C |

This method for the determination of the amino acid diversity number is an all-purpose method and generally applicable and not limited to a specific amino acid sequence, polypeptide, domain or protein. Thus, the proceeding can be applied similarly and accordingly with other sequences to determine and identify amino acid positions with a high variability and which are accessible for alteration without having a strong influence on the stability and functionality of the structure.

With the amino acid diversity calculation amino acid positions with a low diversity number, i.e. smaller than 6, have been identified. This low diversity number resembles a high conservation like e.g. for the cysteine residue in position Nr. 4 (see table III), which was found to be conserved in 57 of the 60 sequences analyzed (see table IV). The cysteine residue in position Nr. 210 was found to be conserved in all analyzed hemopexin-like sequences. This demonstrates the excellent applicability of this approach, as these two cysteine residues are of high importance in the scaffold. These two residues form the disulphide bond that is essential for the formation of the hemopexin-like structure by linking the fourth blade with the first blade of the polypeptide.

From the amino acid diversity number as compiled and listed table III amino acid positions in the consensus sequence with a high diversity/variability can be identified. Because from the identified high diversity amino acid numbers of the consensus sequence the corresponding amino acid numbers of the full length polypeptide cannot be obtained directly, table V lists the amino acid numbers of the identified high diversity amino acids, i.e. alterable amino acids, of the full length polypeptides of SEQ ID NO:02 to SEQ ID NO:61.

TABLE V
Listing of the alterable amino acid positions in each of the sequences SEQ
ID NO: 02 to sequence SEQ ID NO: 61. The numbering of the positions in each
sequence is in consistency with the amino acid numbering of the corresponding
full length protein.

| | | | | | | | | | | |
|---|---|---|---|---|---|---|---|---|---|---|
| SEQ ID NO: 02: | 470 | 471 | 475 | 476 | 477 | 484 | 501 | 502 | 503 | 504 |
| | 505 | 506 | 507 | 510 | 514 | 515 | 522 | 529 | 530 | 531 |
| | 534 | 541 | 547 | 549 | 550 | 553 | 558 | 559 | 566 | 567 |
| | 568 | 569 | 570 | 577 | 578 | 579 | 580 | 589 | 590 | 597 |
| | 598 | 600 | 604 | 608 | 611 | 612 | 617 | 618 | 619 | 620 |
| | 626 | 627 | 628 | 629 | 638 | 639 | 645 | 646 | 647 | 648 |
| | 650 | 652 | 654 | 655 | 658 | 663 | | | | |
| SEQ ID NO: 03: | 98 | 99 | 103 | 104 | 105 | 111 | 128 | 131 | 135 | 136 |
| | 145 | 146 | 153 | 159 | 161 | 162 | 165 | 170 | 171 | 179 |
| | 180 | 181 | 182 | 183 | 190 | 191 | 192 | 193 | 202 | 203 |
| | 210 | 211 | 213 | 217 | 221 | 224 | 225 | 230 | 231 | 232 |
| | 233 | 239 | 240 | 241 | 242 | 251 | 252 | 258 | 259 | 260 |
| | 261 | 266 | 268 | 270 | 271 | 274 | 279 | | | |
| SEQ ID NO: 04: | 98 | 99 | 103 | 104 | 105 | 111 | 128 | 131 | 135 | 136 |
| | 145 | 146 | 153 | 159 | 161 | 162 | 165 | 170 | 171 | 179 |
| | 180 | 181 | 182 | 183 | 190 | 191 | 192 | 193 | 202 | 203 |
| | 210 | 211 | 213 | 217 | 221 | 224 | 225 | 230 | 231 | 232 |
| | 233 | 239 | 240 | 241 | 242 | 251 | 252 | 258 | 259 | 260 |
| | 261 | 266 | 268 | 270 | 271 | 274 | 279 | | | |
| SEQ ID NO: 05: | 469 | 470 | 474 | 475 | 476 | 483 | 500 | 501 | 502 | 503 |
| | 504 | 505 | 506 | 509 | 513 | 514 | 521 | 528 | 529 | 530 |
| | 533 | 540 | 546 | 548 | 549 | 552 | 557 | 558 | 565 | 566 |
| | 567 | 568 | 569 | 576 | 577 | 578 | 579 | 588 | 589 | 596 |
| | 597 | 599 | 603 | 607 | 610 | 611 | 616 | 617 | 618 | 619 |
| | 625 | 626 | 627 | 628 | 637 | 638 | 644 | 645 | 646 | 647 |
| | 649 | 651 | 653 | 654 | 657 | 662 | | | | |
| SEQ ID NO: 06: | 276 | 277 | 281 | 282 | 283 | 290 | 307 | 308 | 309 | 310 |
| | 311 | 312 | 315 | 319 | 320 | 327 | 334 | 335 | 336 | 339 |
| | 346 | 352 | 354 | 355 | 358 | 363 | 364 | 372 | 373 | 374 |
| | 375 | 376 | 383 | 384 | 385 | 386 | 395 | 396 | 403 | 404 |
| | 406 | 410 | 414 | 417 | 418 | 423 | 424 | 425 | 426 | 432 |
| | 433 | 442 | 443 | 449 | 450 | 451 | 452 | 456 | 458 | 460 |
| | 461 | 464 | 468 | | | | | | | |
| SEQ ID NO: 07: | 276 | 277 | 281 | 282 | 283 | 290 | 307 | 308 | 309 | 310 |
| | 311 | 312 | 315 | 319 | 320 | 327 | 334 | 335 | 336 | 339 |
| | 346 | 352 | 354 | 355 | 358 | 363 | 364 | 372 | 373 | 374 |
| | 375 | 376 | 383 | 384 | 385 | 386 | 395 | 396 | 403 | 404 |
| | 406 | 410 | 414 | 417 | 418 | 423 | 424 | 425 | 426 | 432 |
| | 433 | 442 | 443 | 449 | 450 | 451 | 452 | 456 | 458 | 460 |
| | 461 | 464 | 468 | | | | | | | |
| SEQ ID NO: 08: | 276 | 277 | 281 | 282 | 283 | 290 | 307 | 308 | 309 | 310 |
| | 311 | 312 | 315 | 319 | 320 | 327 | 334 | 335 | 336 | 339 |
| | 346 | 352 | 354 | 355 | 358 | 363 | 364 | 372 | 373 | 374 |
| | 375 | 376 | 383 | 384 | 385 | 386 | 395 | 396 | 403 | 404 |
| | 406 | 410 | 414 | 417 | 418 | 423 | 424 | 425 | 426 | 432 |
| | 433 | 442 | 443 | 449 | 450 | 451 | 452 | 456 | 458 | 460 |
| | 461 | 464 | 468 | | | | | | | |
| SEQ ID NO: 09: | 276 | 277 | 281 | 282 | 283 | 290 | 307 | 308 | 309 | 310 |
| | 311 | 312 | 315 | 319 | 320 | 327 | 334 | 335 | 336 | 339 |
| | 346 | 352 | 354 | 355 | 358 | 363 | 364 | 372 | 373 | 374 |
| | 375 | 376 | 383 | 384 | 385 | 386 | 395 | 396 | 403 | 404 |
| | 406 | 410 | 414 | 417 | 418 | 423 | 424 | 425 | 426 | 432 |
| | 433 | 442 | 443 | 449 | 450 | 451 | 452 | 456 | 458 | 460 |
| | 461 | 464 | 468 | | | | | | | |
| SEQ ID NO: 10: | 467 | 468 | 472 | 473 | 474 | 481 | 498 | 499 | 500 | 501 |
| | 502 | 503 | 504 | 507 | 511 | 512 | 519 | 526 | 527 | 528 |
| | 529 | 531 | 538 | 544 | 546 | 547 | 550 | 555 | 556 | 563 |
| | 564 | 565 | 566 | 567 | 574 | 575 | 576 | 577 | 578 | 586 |
| | 587 | 594 | 595 | 596 | 597 | 601 | 605 | 608 | 609 | 614 |
| | 615 | 616 | 617 | 623 | 624 | 625 | 626 | 635 | 636 | 642 |
| | 643 | 644 | 645 | 647 | 649 | 651 | 652 | 655 | 660 | |
| SEQ ID NO: 11: | 288 | 289 | 293 | 294 | 295 | 302 | 319 | 320 | 321 | 322 |
| | 323 | 324 | 327 | 331 | 332 | 339 | 346 | 347 | 348 | 351 |
| | 358 | 364 | 366 | 367 | 370 | 375 | 376 | 383 | 384 | 385 |
| | 386 | 387 | 394 | 395 | 396 | 397 | 406 | 407 | 414 | 415 |
| | 417 | 421 | 425 | 428 | 429 | 434 | 435 | 436 | 437 | 443 |
| | 444 | 454 | 455 | 461 | 462 | 463 | 464 | 467 | 469 | 471 |
| | 472 | 475 | 479 | | | | | | | |
| SEQ ID NO: 12: | 288 | 289 | 293 | 294 | 295 | 302 | 319 | 320 | 321 | 322 |
| | 323 | 324 | 327 | 331 | 332 | 339 | 346 | 347 | 348 | 351 |
| | 358 | 364 | 366 | 367 | 370 | 375 | 376 | 383 | 384 | 385 |
| | 386 | 387 | 394 | 395 | 396 | 397 | 406 | 407 | 414 | 415 |
| | 417 | 421 | 425 | 428 | 429 | 434 | 435 | 436 | 437 | 443 |
| | 444 | 454 | 455 | 461 | 462 | 463 | 464 | 467 | 469 | 471 |
| | 472 | 475 | 479 | | | | | | | |
| SEQ ID NO: 13: | 289 | 290 | 294 | 295 | 296 | 303 | 320 | 321 | 322 | 323 |
| | 324 | 325 | 328 | 332 | 333 | 340 | 347 | 348 | 349 | 352 |
| | 359 | 365 | 367 | 368 | 371 | 376 | 377 | 384 | 385 | 386 |
| | 387 | 388 | 395 | 396 | 397 | 398 | 407 | 408 | 415 | 416 |
| | 418 | 422 | 426 | 429 | 430 | 435 | 436 | 437 | 438 | 444 |
| | 445 | 455 | 456 | 462 | 463 | 464 | 465 | 468 | 470 | 472 |
| | 473 | 476 | 480 | | | | | | | |
| SEQ ID NO: 14: | 286 | 287 | 291 | 292 | 293 | 300 | 317 | 318 | 319 | 320 |
| | 321 | 322 | 325 | 329 | 330 | 337 | 344 | 345 | 346 | 349 |
| | 356 | 362 | 364 | 365 | 368 | 373 | 374 | 381 | 382 | 383 |
| | 384 | 385 | 392 | 393 | 394 | 395 | 404 | 405 | 412 | 413 |
| | 415 | 419 | 423 | 426 | 427 | 432 | 433 | 434 | 435 | 441 |
| | 442 | 452 | 453 | 459 | 460 | 461 | 462 | 465 | 467 | 469 |
| | 470 | 473 | 477 | | | | | | | |
| SEQ ID NO: 15: | 277 | 278 | 282 | 283 | 284 | 291 | 308 | 309 | 310 | 311 |
| | 312 | 313 | 316 | 320 | 321 | 328 | 335 | 336 | 337 | 340 |
| | 347 | 353 | 355 | 356 | 359 | 364 | 365 | 372 | 373 | 374 |
| | 375 | 376 | 383 | 384 | 394 | 395 | 402 | 403 | 405 | 409 |
| | 413 | 416 | 417 | 422 | 423 | 424 | 425 | 431 | 432 | 441 |
| | 442 | 448 | 449 | 450 | 451 | 453 | 455 | 457 | 458 | 461 |
| | 465 | | | | | | | | | |
| SEQ ID NO: 16: | 277 | 278 | 282 | 283 | 284 | 291 | 308 | 309 | 310 | 311 |
| | 312 | 313 | 316 | 320 | 321 | 328 | 335 | 336 | 337 | 340 |
| | 347 | 353 | 355 | 356 | 359 | 364 | 365 | 372 | 373 | 374 |
| | 375 | 376 | 383 | 384 | 394 | 395 | 402 | 403 | 405 | 409 |
| | 413 | 416 | 417 | 422 | 423 | 424 | 425 | 431 | 432 | 441 |
| | 442 | 448 | 449 | 450 | 451 | 453 | 455 | 457 | 458 | 461 |
| | 465 | | | | | | | | | |
| SEQ ID NO: 17: | 278 | 279 | 283 | 284 | 285 | 292 | 309 | 310 | 311 | 312 |
| | 313 | 314 | 317 | 321 | 322 | 329 | 336 | 337 | 338 | 341 |
| | 348 | 354 | 356 | 357 | 360 | 365 | 366 | 373 | 374 | 375 |
| | 376 | 377 | 384 | 385 | 395 | 396 | 403 | 404 | 406 | 410 |
| | 414 | 417 | 418 | 423 | 424 | 425 | 426 | 432 | 433 | 442 |
| | 443 | 449 | 450 | 451 | 452 | 454 | 456 | 458 | 459 | 462 |
| | 466 | | | | | | | | | |
| SEQ ID NO: 18: | 519 | 520 | 524 | 525 | 532 | 550 | 551 | 552 | 553 | 554 |
| | 555 | 556 | 559 | 563 | 564 | 571 | 578 | 579 | 580 | 583 |
| | 590 | 596 | 597 | 598 | 601 | 605 | 606 | 613 | 614 | 615 |
| | 616 | 617 | 624 | 625 | 626 | 635 | 636 | 643 | 644 | 646 |
| | 650 | 654 | 657 | 658 | 663 | 664 | 665 | 666 | 672 | 673 |
| | 674 | 682 | 683 | 689 | 690 | 691 | 692 | 694 | 696 | 698 |
| | 699 | 702 | 707 | | | | | | | |
| SEQ ID NO: 19: | 511 | 512 | 516 | 517 | 524 | 542 | 543 | 544 | 545 | 546 |
| | 547 | 548 | 551 | 555 | 556 | 563 | 570 | 571 | 572 | 575 |
| | 582 | 588 | 589 | 590 | 593 | 597 | 598 | 605 | 606 | 607 |
| | 608 | 609 | 616 | 617 | 618 | 627 | 628 | 635 | 636 | 638 |
| | 642 | 646 | 649 | 650 | 655 | 656 | 657 | 658 | 664 | 665 |
| | 666 | 674 | 675 | 681 | 682 | 683 | 684 | 686 | 688 | 690 |
| | 691 | 694 | 699 | | | | | | | |
| SEQ ID NO: 20: | 514 | 515 | 519 | 520 | 527 | 545 | 546 | 547 | 548 | 549 |
| | 550 | 551 | 554 | 558 | 559 | 566 | 573 | 574 | 575 | 578 |
| | 585 | 591 | 592 | 593 | 596 | 600 | 601 | 608 | 609 | 610 |
| | 611 | 612 | 619 | 620 | 621 | 630 | 631 | 638 | 639 | 641 |
| | 645 | 649 | 652 | 653 | 658 | 659 | 660 | 661 | 667 | 668 |
| | 669 | 677 | 678 | 684 | 685 | 686 | 687 | 695 | 698 | 700 |
| | 701 | 704 | 709 | | | | | | | |
| SEQ ID NO: 21: | 532 | 533 | 537 | 538 | 545 | 563 | 564 | 565 | 566 | 567 |
| | 568 | 569 | 572 | 576 | 577 | 584 | 591 | 592 | 593 | 596 |
| | 603 | 609 | 610 | 611 | 614 | 618 | 619 | 626 | 627 | 628 |
| | 629 | 630 | 637 | 638 | 639 | 648 | 649 | 656 | 657 | 659 |
| | 663 | 667 | 670 | 671 | 676 | 677 | 678 | 679 | 685 | 686 |
| | 687 | 695 | 696 | 702 | 703 | 704 | 705 | 707 | 709 | 711 |
| | 712 | 715 | 720 | | | | | | | |
| SEQ ID NO: 22: | 287 | 288 | 291 | 294 | 301 | 316 | 317 | 318 | 319 | 320 |
| | 321 | 324 | 328 | 329 | 336 | 343 | 344 | 345 | 348 | 355 |
| | 361 | 363 | 364 | 367 | 372 | 373 | 380 | 381 | 382 | 383 |
| | 384 | 391 | 392 | 393 | 394 | 403 | 404 | 411 | 412 | 414 |
| | 418 | 422 | 425 | 426 | 431 | 432 | 433 | 434 | 440 | 441 |
| | 450 | 451 | 457 | 458 | 459 | 460 | 462 | 464 | 466 | 467 |
| | 470 | 474 | | | | | | | | |
| SEQ ID NO: 23: | 287 | 288 | 291 | 294 | 301 | 316 | 317 | 318 | 319 | 320 |
| | 321 | 324 | 328 | 329 | 336 | 343 | 344 | 345 | 348 | 355 |
| | 361 | 363 | 364 | 367 | 372 | 373 | 380 | 381 | 382 | 383 |
| | 384 | 391 | 392 | 393 | 394 | 403 | 404 | 411 | 412 | 414 |
| | 418 | 422 | 425 | 426 | 431 | 432 | 433 | 434 | 440 | 441 |
| | 450 | 451 | 457 | 458 | 459 | 460 | 462 | 464 | 466 | 467 |
| | 470 | 474 | | | | | | | | |
| SEQ ID NO: 24: | 292 | 293 | 296 | 297 | 304 | 322 | 323 | 324 | 325 | 326 |
| | 327 | 330 | 334 | 335 | 342 | 349 | 350 | 351 | 353 | 360 |
| | 366 | 368 | 369 | 372 | 376 | 377 | 384 | 385 | 386 | 387 |
| | 388 | 395 | 396 | 397 | 406 | 407 | 414 | 415 | 417 | 420 |
| | 424 | 427 | 428 | 433 | 434 | 435 | 436 | 442 | 443 | 444 |
| | 445 | 453 | 454 | 460 | 461 | 462 | 463 | 465 | 467 | 469 |
| | 470 | 473 | 478 | | | | | | | |
| SEQ ID NO: 25: | 296 | 297 | 300 | 301 | 308 | 326 | 327 | 328 | 329 | 330 |
| | 331 | 334 | 338 | 339 | 346 | 353 | 354 | 355 | 357 | 364 |
| | 370 | 372 | 373 | 376 | 380 | 381 | 388 | 389 | 390 | 391 |
| | 392 | 399 | 400 | 401 | 410 | 411 | 418 | 419 | 421 | 424 |
| | 428 | 431 | 432 | 437 | 438 | 439 | 440 | 446 | 447 | 448 |
| | 449 | 457 | 458 | 464 | 465 | 466 | 467 | 469 | 471 | 473 |
| | 474 | 477 | 482 | | | | | | | |
| SEQ ID NO: 26: | 280 | 281 | 285 | 286 | 287 | 294 | 311 | 312 | 313 | 314 |
| | 315 | 316 | 319 | 323 | 324 | 331 | 338 | 339 | 340 | 343 |
| | 350 | 356 | 358 | 359 | 362 | 367 | 368 | 375 | 376 | 377 |
| | 378 | 379 | 386 | 387 | 388 | 389 | 398 | 399 | 406 | 407 |
| | 409 | 413 | 417 | 420 | 421 | 426 | 427 | 428 | 429 | 435 |
| | 436 | 437 | 446 | 447 | 453 | 454 | 455 | 456 | 458 | 460 |
| | 462 | 463 | 466 | 470 | | | | | | |
| SEQ ID NO: 27: | 273 | 274 | 278 | 279 | 280 | 287 | 304 | 305 | 306 | 307 |
| | 308 | 309 | 312 | 316 | 317 | 324 | 331 | 332 | 333 | 336 |
| | 343 | 349 | 351 | 352 | 355 | 360 | 361 | 368 | 369 | 370 |
| | 371 | 372 | 379 | 380 | 381 | 382 | 391 | 392 | 399 | 400 |
| | 402 | 406 | 410 | 413 | 414 | 419 | 420 | 421 | 422 | 428 |
| | 429 | 438 | 439 | 445 | 446 | 447 | 448 | 450 | 452 | 454 |
| | 455 | 458 | 462 | | | | | | | |
| SEQ ID NO: 28: | 275 | 276 | 280 | 281 | 282 | 289 | 306 | 307 | 308 | 309 |
| | 310 | 311 | 314 | 318 | 319 | 326 | 333 | 334 | 335 | 338 |
| | 345 | 351 | 353 | 354 | 357 | 362 | 363 | 370 | 371 | 372 |
| | 373 | 374 | 381 | 382 | 383 | 384 | 393 | 394 | 401 | 402 |
| | 404 | 408 | 412 | 415 | 416 | 421 | 422 | 423 | 424 | 430 |
| | 431 | 440 | 441 | 447 | 448 | 449 | 450 | 452 | 454 | 456 |
| | 457 | 460 | 464 | | | | | | | |
| SEQ ID NO: 29: | 276 | 277 | 281 | 282 | 283 | 290 | 307 | 308 | 309 | 310 |
| | 311 | 312 | 315 | 319 | 320 | 327 | 334 | 335 | 336 | 339 |
| | 346 | 352 | 354 | 355 | 358 | 363 | 364 | 371 | 372 | 373 |
| | 374 | 375 | 382 | 383 | 384 | 385 | 394 | 395 | 402 | 403 |
| | 405 | 409 | 413 | 416 | 417 | 422 | 423 | 424 | 425 | 431 |
| | 432 | 441 | 442 | 448 | 449 | 450 | 451 | 453 | 455 | 457 |
| | 458 | 461 | 465 | | | | | | | |
| SEQ ID NO: 30: | 282 | 283 | 287 | 288 | 289 | 296 | 313 | 314 | 315 | 316 |
| | 317 | 318 | 321 | 325 | 326 | 333 | 340 | 341 | 342 | 345 |
| | 352 | 358 | 360 | 361 | 364 | 369 | 370 | 377 | 378 | 379 |
| | 380 | 381 | 388 | 389 | 390 | 391 | 400 | 401 | 408 | 409 |
| | 411 | 415 | 419 | 422 | 423 | 428 | 429 | 430 | 431 | 437 |
| | 438 | 447 | 448 | 454 | 455 | 456 | 457 | 459 | 461 | 463 |
| | 464 | 467 | 471 | | | | | | | |
| SEQ ID NO: 31: | 283 | 284 | 288 | 289 | 290 | 297 | 314 | 315 | 316 | 317 |
| | 318 | 319 | 322 | 326 | 327 | 334 | 341 | 342 | 343 | 346 |
| | 353 | 359 | 361 | 362 | 365 | 370 | 371 | 378 | 379 | 380 |
| | 381 | 382 | 389 | 390 | 391 | 392 | 401 | 402 | 409 | 410 |
| | 412 | 416 | 420 | 423 | 424 | 429 | 430 | 431 | 432 | 438 |
| | 439 | 448 | 449 | 455 | 456 | 457 | 458 | 460 | 462 | 464 |
| | 465 | 468 | 472 | | | | | | | |
| SEQ ID NO: 32: | 282 | 283 | 287 | 288 | 289 | 296 | 313 | 314 | 315 | 316 |
| | 317 | 318 | 321 | 325 | 326 | 333 | 340 | 341 | 342 | 345 |
| | 352 | 358 | 360 | 361 | 364 | 369 | 370 | 377 | 378 | 379 |
| | 380 | 381 | 388 | 389 | 390 | 391 | 400 | 401 | 408 | 409 |
| | 411 | 415 | 419 | 422 | 423 | 428 | 429 | 430 | 431 | 437 |
| | 438 | 447 | 448 | 454 | 455 | 456 | 457 | 459 | 461 | 463 |
| | 464 | 467 | 471 | | | | | | | |
| SEQ ID NO: 33: | 282 | 283 | 287 | 288 | 289 | 296 | 313 | 314 | 315 | 316 |
| | 317 | 318 | 321 | 325 | 326 | 333 | 340 | 341 | 342 | 345 |
| | 352 | 358 | 360 | 361 | 364 | 369 | 370 | 377 | 378 | 379 |
| | 380 | 381 | 388 | 389 | 390 | 391 | 400 | 401 | 408 | 409 |
| | 411 | 415 | 419 | 422 | 423 | 428 | 429 | 430 | 431 | 437 |
| | 438 | 447 | 448 | 454 | 455 | 456 | 457 | 459 | 461 | 463 |
| | 464 | 467 | 471 | | | | | | | |
| SEQ ID NO: 34: | 317 | 318 | 322 | 329 | 347 | 348 | 349 | 350 | 351 | 352 |
| | 355 | 359 | 360 | 367 | 374 | 375 | 378 | 385 | 391 | 393 |
| | 394 | 397 | 402 | 403 | 411 | 412 | 413 | 414 | 421 | 422 |
| | 423 | 424 | 433 | 434 | 441 | 442 | 444 | 448 | 452 | 454 |
| | 455 | 460 | 461 | 462 | 463 | 469 | 470 | 471 | 472 | 481 |
| | 482 | 488 | 489 | 490 | 491 | 493 | 495 | 497 | 498 | 501 |
| | 506 | | | | | | | | | |
| SEQ ID NO: 35: | 317 | 318 | 322 | 329 | 347 | 348 | 349 | 350 | 351 | 352 |
| | 355 | 359 | 360 | 367 | 374 | 375 | 378 | 385 | 391 | 393 |
| | 394 | 397 | 402 | 403 | 411 | 412 | 413 | 414 | 421 | 422 |
| | 423 | 424 | 433 | 434 | 441 | 442 | 444 | 448 | 452 | 454 |
| | 455 | 460 | 461 | 462 | 463 | 469 | 470 | 471 | 472 | 481 |
| | 482 | 488 | 489 | 490 | 491 | 493 | 495 | 497 | 498 | 501 |
| | 506 | | | | | | | | | |
| SEQ ID NO: 36: | 315 | 316 | 320 | 327 | 345 | 346 | 347 | 348 | 349 | 350 |
| | 353 | 357 | 358 | 365 | 372 | 373 | 376 | 383 | 389 | 391 |
| | 392 | 395 | 400 | 401 | 409 | 410 | 411 | 412 | 419 | 420 |
| | 421 | 422 | 431 | 432 | 439 | 440 | 442 | 446 | 450 | 452 |
| | 453 | 458 | 459 | 460 | 461 | 467 | 468 | 469 | 470 | 479 |
| | 480 | 486 | 487 | 488 | 489 | 491 | 493 | 495 | 496 | 499 |
| | 504 | | | | | | | | | |
| SEQ ID NO: 37: | 317 | 318 | 322 | 329 | 347 | 348 | 349 | 350 | 351 | 352 |
| | 355 | 359 | 360 | 367 | 374 | 375 | 378 | 385 | 391 | 393 |
| | 394 | 397 | 402 | 403 | 411 | 412 | 413 | 414 | 421 | 422 |
| | 423 | 424 | 433 | 434 | 441 | 442 | 444 | 448 | 452 | 454 |
| | 455 | 460 | 461 | 462 | 463 | 469 | 470 | 471 | 472 | 481 |
| | 482 | 488 | 489 | 490 | 491 | 493 | 495 | 497 | 498 | 501 |
| | 506 | | | | | | | | | |
| SEQ ID NO: 38: | 317 | 318 | 322 | 329 | 347 | 348 | 349 | 350 | 351 | 352 |
| | 355 | 359 | 360 | 367 | 374 | 375 | 378 | 385 | 391 | 393 |
| | 394 | 397 | 402 | 403 | 411 | 412 | 413 | 414 | 421 | 422 |
| | 423 | 424 | 433 | 434 | 441 | 442 | 444 | 448 | 452 | 454 |
| | 455 | 460 | 461 | 462 | 463 | 469 | 470 | 471 | 472 | 481 |
| | 482 | 488 | 489 | 490 | 491 | 493 | 495 | 497 | 498 | 501 |
| | 506 | | | | | | | | | |
| SEQ ID NO: 39: | 368 | 369 | 373 | 380 | 398 | 399 | 400 | 401 | 402 | 403 |
| | 406 | 410 | 411 | 418 | 425 | 426 | 429 | 436 | 442 | 444 |
| | 445 | 448 | 453 | 454 | 462 | 463 | 464 | 465 | 472 | 473 |
| | 474 | 475 | 484 | 485 | 492 | 493 | 495 | 499 | 503 | 505 |
| | 506 | 511 | 512 | 513 | 514 | 520 | 521 | 522 | 523 | 532 |
| | 533 | 539 | 540 | 541 | 542 | 544 | 546 | 548 | 549 | 552 |
| | 557 | | | | | | | | | |
| SEQ ID NO: 40: | 364 | 365 | 369 | 376 | 394 | 395 | 396 | 397 | 398 | 399 |
| | 402 | 406 | 407 | 415 | 422 | 423 | 426 | 433 | 439 | 441 |
| | 442 | 445 | 450 | 451 | 459 | 460 | 461 | 462 | 469 | 470 |
| | 471 | 472 | 481 | 482 | 489 | 490 | 492 | 496 | 500 | 502 |
| | 503 | 508 | 509 | 510 | 511 | 517 | 518 | 519 | 520 | 529 |
| | 530 | 536 | 537 | 538 | 539 | 541 | 543 | 545 | 546 | 549 |
| | 554 | | | | | | | | | |
| SEQ ID NO: 41: | 341 | 342 | 346 | 353 | 371 | 372 | 373 | 374 | 375 | 376 |
| | 379 | 383 | 384 | 391 | 398 | 399 | 402 | 409 | 415 | 417 |
| | 418 | 421 | 426 | 427 | 435 | 436 | 437 | 438 | 445 | 446 |
| | 447 | 448 | 457 | 458 | 465 | 466 | 468 | 472 | 476 | 478 |
| | 479 | 484 | 485 | 486 | 487 | 493 | 494 | 495 | 496 | 505 |
| | 506 | 512 | 513 | 514 | 515 | 517 | 519 | 521 | 522 | 525 |
| | 530 | | | | | | | | | |
| SEQ ID NO: 42: | 341 | 342 | 346 | 353 | 371 | 372 | 373 | 374 | 375 | 376 |
| | 379 | 383 | 384 | 391 | 398 | 399 | 402 | 409 | 415 | 417 |
| | 418 | 421 | 426 | 427 | 435 | 436 | 437 | 438 | 445 | 446 |
| | 447 | 448 | 457 | 458 | 465 | 466 | 468 | 472 | 476 | 478 |
| | 479 | 484 | 485 | 486 | 487 | 493 | 494 | 495 | 496 | 505 |
| | 506 | 512 | 513 | 514 | 515 | 517 | 519 | 521 | 522 | 525 |
| | 530 | | | | | | | | | |
| SEQ ID NO: 43: | 341 | 342 | 346 | 353 | 371 | 372 | 373 | 374 | 375 | 376 |
| | 379 | 383 | 384 | 391 | 398 | 399 | 402 | 409 | 415 | 417 |
| | 418 | 421 | 426 | 427 | 435 | 436 | 437 | 438 | 445 | 446 |
| | 447 | 448 | 457 | 458 | 465 | 466 | 468 | 472 | 476 | 478 |
| | 479 | 484 | 485 | 486 | 487 | 493 | 494 | 495 | 496 | 505 |
| | 506 | 512 | 513 | 514 | 515 | 517 | 519 | 521 | 522 | 525 |
| | 530 | | | | | | | | | |
| SEQ ID NO: 44: | 333 | 334 | 338 | 345 | 363 | 364 | 365 | 366 | 367 | 368 |
| | 369 | 372 | 376 | 377 | 387 | 394 | 395 | 396 | 399 | 406 |
| | 412 | 414 | 415 | 418 | 423 | 424 | 430 | 431 | 432 | 433 |
| | 440 | 441 | 442 | 443 | 452 | 453 | 460 | 461 | 463 | 467 |
| | 471 | 473 | 474 | 479 | 480 | 481 | 482 | 488 | 489 | 490 |
| | 491 | 499 | 500 | 506 | 507 | 508 | 509 | 511 | 513 | 515 |
| | 516 | 519 | 524 | | | | | | | |
| SEQ ID NO: 45: | 334 | 335 | 339 | 346 | 364 | 365 | 366 | 367 | 368 | 369 |
| | 370 | 373 | 377 | 378 | 388 | 395 | 396 | 397 | 400 | 407 |
| | 413 | 415 | 416 | 419 | 424 | 425 | 431 | 432 | 433 | 434 |
| | 441 | 442 | 443 | 444 | 453 | 454 | 461 | 462 | 464 | 468 |
| | 472 | 474 | 475 | 480 | 481 | 482 | 483 | 489 | 490 | 491 |
| | 492 | 500 | 501 | 507 | 508 | 509 | 510 | 512 | 514 | 516 |
| | 517 | 520 | 525 | | | | | | | |
| SEQ ID NO: 46: | 278 | 279 | 283 | 284 | 285 | 292 | 309 | 310 | 311 | 312 |
| | 313 | 314 | 317 | 321 | 322 | 329 | 336 | 337 | 338 | 341 |
| | 348 | 354 | 356 | 357 | 360 | 365 | 366 | 373 | 374 | 375 |
| | 376 | 377 | 384 | 385 | 386 | 387 | 396 | 397 | 404 | 405 |
| | 407 | 411 | 415 | 418 | 419 | 424 | 425 | 426 | 427 | 433 |
| | 434 | 443 | 444 | 450 | 451 | 452 | 453 | 455 | 457 | 459 |
| | 460 | 463 | 467 | | | | | | | |
| SEQ ID NO: 47: | 287 | 288 | 292 | 293 | 294 | 300 | 318 | 319 | 320 | 321 |
| | 322 | 324 | 328 | 329 | 336 | 343 | 344 | 345 | 348 | 355 |
| | 361 | 363 | 364 | 367 | 372 | 373 | 375 | 376 | 377 | 378 |
| | 379 | 386 | 387 | 388 | 389 | 398 | 399 | 406 | 407 | 409 |
| | 413 | 417 | 420 | 421 | 426 | 427 | 428 | 429 | 435 | 436 |
| | 437 | 437 | 446 | 447 | 453 | 454 | 455 | 456 | 458 | 460 |
| | 462 | 463 | 466 | 471 | | | | | | |
| SEQ ID NO: 48: | 287 | 288 | 292 | 293 | 294 | 300 | 318 | 319 | 320 | 321 |
| | 322 | 324 | 328 | 329 | 336 | 343 | 344 | 345 | 348 | 355 |
| | 361 | 363 | 364 | 367 | 372 | 373 | 375 | 376 | 377 | 378 |
| | 379 | 386 | 387 | 388 | 389 | 398 | 399 | 406 | 407 | 409 |
| | 413 | 417 | 420 | 421 | 426 | 427 | 428 | 429 | 435 | 436 |
| | 437 | 446 | 447 | 453 | 454 | 455 | 456 | 458 | 460 | 462 |
| | 463 | 466 | 471 | | | | | | | |
| SEQ ID NO: 49: | 292 | 293 | 297 | 298 | 299 | 306 | 323 | 324 | 325 | 326 |
| | 327 | 328 | 329 | 332 | 336 | 337 | 344 | 351 | 352 | 353 |
| | 356 | 363 | 369 | 371 | 372 | 374 | 379 | 380 | 387 | 388 |
| | 389 | 390 | 391 | 398 | 399 | 400 | 401 | 410 | 411 | 418 |
| | 419 | 421 | 425 | 429 | 432 | 433 | 438 | 439 | 440 | 441 |
| | 447 | 448 | 457 | 458 | 464 | 465 | 466 | 467 | 469 | 471 |
| | 473 | 474 | 477 | 481 | | | | | | |
| SEQ ID NO: 50: | 294 | 295 | 299 | 300 | 301 | 308 | 325 | 326 | 327 | 328 |
| | 329 | 330 | 331 | 334 | 338 | 339 | 346 | 353 | 354 | 355 |
| | 358 | 365 | 371 | 373 | 374 | 376 | 381 | 382 | 389 | 390 |
| | 391 | 392 | 393 | 400 | 401 | 402 | 403 | 412 | 413 | 420 |
| | 421 | 423 | 427 | 431 | 434 | 435 | 440 | 441 | 442 | 443 |
| | 449 | 450 | 459 | 460 | 466 | 467 | 468 | 469 | 471 | 473 |
| | 475 | 476 | 479 | 483 | | | | | | |
| SEQ ID NO: 51: | 293 | 294 | 298 | 299 | 300 | 307 | 324 | 325 | 326 | 327 |
| | 328 | 329 | 330 | 333 | 337 | 338 | 345 | 352 | 353 | 354 |
| | 357 | 364 | 370 | 372 | 373 | 375 | 380 | 381 | 388 | 389 |
| | 390 | 391 | 392 | 399 | 400 | 401 | 402 | 411 | 412 | 419 |
| | 420 | 422 | 426 | 430 | 433 | 434 | 439 | 440 | 441 | 442 |
| | 448 | 449 | 458 | 459 | 465 | 466 | 467 | 468 | 470 | 472 |
| | 474 | 475 | 478 | 482 | | | | | | |
| SEQ ID NO: 52: | 294 | 295 | 299 | 300 | 301 | 308 | 325 | 326 | 327 | 328 |
| | 329 | 330 | 331 | 334 | 338 | 339 | 346 | 353 | 354 | 355 |
| | 358 | 365 | 371 | 373 | 374 | 376 | 381 | 382 | 389 | 390 |
| | 391 | 392 | 393 | 400 | 401 | 402 | 403 | 412 | 413 | 420 |
| | 421 | 423 | 427 | 431 | 434 | 435 | 440 | 441 | 442 | 443 |
| | 449 | 450 | 459 | 460 | 466 | 467 | 468 | 469 | 471 | 473 |
| | 475 | 476 | 479 | 483 | | | | | | |
| SEQ ID NO: 53: | 378 | 379 | 383 | 390 | 408 | 409 | 410 | 411 | 412 | 413 |
| | 416 | 420 | 421 | 428 | 435 | 436 | 439 | 446 | 452 | 454 |
| | 455 | 458 | 463 | 464 | 472 | 473 | 474 | 475 | 482 | 483 |
| | 484 | 485 | 494 | 495 | 502 | 503 | 505 | 509 | 513 | 515 |
| | 516 | 521 | 522 | 523 | 524 | 530 | 531 | 532 | 533 | 542 |
| | 543 | 549 | 550 | 551 | 552 | 554 | 556 | 558 | 559 | 562 |
| | 567 | | | | | | | | | |
| SEQ ID NO: 54: | 351 | 352 | 356 | 363 | 381 | 382 | 383 | 384 | 385 | 386 |
| | 389 | 393 | 394 | 401 | 408 | 409 | 412 | 419 | 425 | 427 |
| | 428 | 431 | 436 | 437 | 445 | 446 | 447 | 448 | 455 | 456 |
| | 457 | 458 | 467 | 468 | 475 | 476 | 478 | 482 | 486 | 488 |
| | 489 | 494 | 495 | 496 | 497 | 503 | 504 | 505 | 506 | 515 |
| | 516 | 522 | 523 | 524 | 525 | 527 | 529 | 531 | 532 | 535 |
| | 540 | | | | | | | | | |
| SEQ ID NO: 55: | 351 | 352 | 356 | 363 | 381 | 382 | 383 | 384 | 385 | 386 |
| | 389 | 393 | 394 | 401 | 408 | 409 | 412 | 419 | 425 | 427 |
| | 428 | 431 | 436 | 437 | 445 | 446 | 447 | 448 | 455 | 456 |
| | 457 | 458 | 467 | 468 | 475 | 476 | 478 | 482 | 486 | 488 |
| | 489 | 494 | 495 | 496 | 497 | 503 | 504 | 505 | 506 | 515 |
| | 516 | 522 | 523 | 524 | 525 | 527 | 529 | 531 | 532 | 535 |
| | 540 | | | | | | | | | |
| SEQ ID NO: 56: | 315 | 316 | 320 | 327 | 345 | 346 | 347 | 348 | 349 | 350 |
| | 351 | 354 | 358 | 359 | 369 | 376 | 377 | 378 | 381 | 388 |
| | 394 | 396 | 397 | 400 | 404 | 405 | 411 | 412 | 413 | 414 |
| | 415 | 422 | 423 | 424 | 425 | 434 | 435 | 442 | 443 | 445 |
| | 449 | 453 | 455 | 456 | 461 | 462 | 463 | 464 | 470 | 471 |
| | 472 | 473 | 481 | 482 | 488 | 489 | 490 | 491 | 493 | 495 |
| | 497 | 498 | 501 | 506 | | | | | | |
| SEQ ID NO: 57: | 327 | 334 | 353 | 354 | 355 | 356 | 357 | 360 | 364 | 365 |
| | 372 | 379 | 380 | 381 | 384 | 391 | 397 | 399 | 400 | 403 |
| | 408 | 409 | 413 | 414 | 415 | 416 | 417 | 424 | 425 | 426 |
| | 427 | 436 | 437 | 444 | 445 | 447 | 451 | 455 | 457 | 458 |
| | 463 | 464 | 465 | 466 | 472 | 473 | 474 | 483 | 484 | 490 |
| | 491 | 492 | 493 | 495 | 497 | 499 | 500 | 503 | 508 | |
| SEQ ID NO: 58: | 291 | 292 | 296 | 297 | 298 | 305 | 323 | 324 | 325 | 326 |
| | 327 | 330 | 334 | 335 | 341 | 345 | 346 | 347 | 350 | 357 |
| | 363 | 365 | 366 | 369 | 374 | 375 | 383 | 384 | 385 | 386 |
| | 387 | 390 | 391 | 392 | 393 | 400 | 407 | 408 | 410 | 414 |
| | 418 | 421 | 422 | 427 | 428 | 429 | 430 | 432 | 439 | 440 |
| | 446 | 447 | 448 | 449 | 453 | 456 | 458 | 459 | 462 | 467 |
| SEQ ID NO: 59: | 290 | 291 | 295 | 296 | 297 | 304 | 322 | 323 | 324 | 325 |
| | 326 | 329 | 333 | 334 | 340 | 344 | 345 | 346 | 349 | 356 |
| | 362 | 364 | 365 | 368 | 373 | 374 | 382 | 383 | 384 | 385 |
| | 386 | 391 | 392 | 393 | 394 | 401 | 408 | 409 | 411 | 415 |
| | 419 | 422 | 423 | 428 | 429 | 430 | 431 | 433 | | 440 |
| | 441 | 447 | 448 | 449 | 450 | 454 | 457 | 459 | 460 | 463 |
| | 468 | | | | | | | | | |
| SEQ ID NO: 60: | 268 | 269 | 273 | 274 | 275 | 282 | 300 | 301 | 302 | 303 |
| | 304 | 307 | 311 | 312 | 318 | 322 | 323 | 324 | 327 | 334 |
| | 340 | 342 | 343 | 346 | 351 | 352 | 360 | 361 | 362 | 363 |
| | 364 | 371 | 372 | 373 | 374 | 381 | 388 | 389 | 391 | 395 |
| | 399 | 402 | 403 | 408 | 409 | 410 | 411 | 413 | 420 | 421 |
| | 427 | 428 | 429 | 430 | 434 | 437 | 439 | 440 | 443 | 448 |
| SEQ ID NO: 61: | 291 | 292 | 296 | 297 | 298 | 305 | 323 | 324 | 325 | 326 |
| | 327 | 330 | 334 | 335 | 341 | 345 | 346 | 347 | 350 | 357 |
| | 362 | 364 | 365 | 368 | 373 | 374 | 380 | 381 | 382 | 383 |
| | 384 | 387 | 388 | 389 | 390 | 397 | 404 | 405 | 407 | 411 |
| | 415 | 418 | 419 | 424 | 425 | 426 | 427 | 429 | 436 | 437 |
| | 443 | 444 | 445 | 446 | 450 | 453 | 455 | 456 | 459 | 464 |

(Table V end)

Positions with a high diversity number, i.e. equal or higher than 8, or even 10, have also been determined. The analysis revealed that these are mainly located in loop regions. These expose a high variability, i.e. flexibility, and as a result spatially bring together several surface exposed amino acids from the blade connecting loops. The results also suggest not using the interior surface of the tunnel for randomization experiments. The inner three beta-sheets of each blade were also critical, because they resemble a high conservation and contributed to the core stability of the protein. Thus, solvent-exposed amino acids, which do not contribute to the hydrophobic core stability of the protein, which revealed a sufficient high diversity number and hence a low conservation, are in the focus of interest for a mutagenesis approach.

With this method it is possible to obtain a list of variable, i.e. alterable, amino acid positions in and for all proteins, which have been employed in the alignment, at the same time. For the hemopexin-like domain, as exemplified before, hemopexin-like domains of sixty proteins have been employed and thus for all sixty domains the positions of alterable, i.e. variable, amino acids have been identified. These positions are listed in table V (the numbering is according to the full length polypeptide/protein).

In table V the variable amino acid positions in the sixty hemopexin-like domains (SEQ ID NO:02 to SEQ ID NO:61) are listed. The amino acid positions are numbered according to the full length sequence of the protein containing the hemopexin-like domain. For example, for the hemopexin-like domain according to SEQ ID NO:02 these are the amino acid positions listed after the subheading SEQ ID NO:02 of table V and are accordingly 470, 471, 475, 476, 477, 484, 501, 502, 503, 504, 505, 506, 507, 510, 514, 515, 522, 529, 530, 531, 534, 541, 547, 549, 550, 553, 558, 559, 566, 567, 568, 569, 570, 577, 578, 579, 580, 589, 590, 597, 598, 600, 604, 608, 611, 612, 617, 618, 619, 620, 626, 627, 628, 629, 638, 639, 645, 646, 647, 648, 650, 652, 654, 655, 658, 663. The alterable amino acid positions for SEQ ID NO:03 to SEQ ID NO:61 are accordingly listed in table V after the respective subheading.

With the alterable amino acid positions available for SEQ ID NO:01 to SEQ ID NO:61 each of these sequences can be taken as starting point for further operations.

The cell-free production and analysis of rationally engineered protein variants can be automated, but the library size of rationally designed protein-constructs to be processed remains always limited by the technical throughput of each system. Therefore the analysis of the binding-properties of a vast multitude of gene-products demands for further efforts.

For display and screening of a polypeptide library multiple techniques are available, as e.g. phage-, ribosome- or bacterial-display (Smith, G. P., Science 228 (1985) 1315-1317; Hanes, J., and Pluckthun, A., PNAS 94 (1997) 4937-4942; Stahl, S., and Uhlen, M., TIBTECH 15 (1997) 185-192).

The current invention will be exemplified with the ribosome display technique (see e.g. Hanes, J., and Pluckthun, A., PNAS 94 (1997) 4937-4942; Mattheakis, L. C., et al., PNAS 91 (1994) 9022-9026; He, M., and Taussig, M. J., Nuc. Acids Res. 25 (1997) 5132-5134), but other techniques are also applicable.

Directed evolutionary techniques are well suited to complement the technical capability of a high throughput protein production platform. Based on the cell-free protein synthesis technology, ribosome display is an excellent method to be implemented into a high throughput protein production and analysis process. The aim of ribosome display is the generation of ternary complexes, in which the genotype, characterized by its messenger-RNA (mRNA), is physically linked by the ribosome to its encoded phenotype, characterized by the expressed polypeptides.

For this purpose, a linear DNA-template, which encodes a gene-library is transcribed and translated in vitro. Downstream of the gene-sequence a spacer sequence is fused, where the predominant feature is the lack of a translational stop codon. This spacer domain facilitates the display of the nascent translated and co-translationally folded polypeptide, which remains tethered to the ribosome. These complexes are subjected to a panning procedure, in which the ribosome-displayed polypeptide is allowed to bind to a predetermined ligand molecule. The mRNA from tightly bound complexes is isolated, reversibly transcribed and amplified by PCR. Sub cloning of the PCR products into a vector system and consecutive DNA sequencing reveals information about the genotype related to the phenotype of the bound polypeptide. In repeated cycles of mutagenesis and ribosome display specific protein-binders from libraries in the range of up to 1014 members can be identified (Mattheakis, L. C., et al., PNAS 91 (1994) 9022-9026; Hanes, J., and Pluckthun, A., PNAS 94 (1997) 4937-4942; Lamla, T., and Erdmann, V. A., J. Mol. Biol. 329 (2003) 381-388).

In general, ribosome display requires the stalling of the ribosome while reaching the 3′-end of the mRNA without the dissociation of the ribosomal subunits. After the ribosome has encountered the 3′-end of the mRNA the ribosome's transfer-RNA (tRNA) entry site (A-site) is unoccupied. In prokaryotes, this state results in the activation of the ribosome rescue mechanism, induced by tmRNA (transfer messenger RNA; Abo, T., et al. EMBO J. 19 (2000) 3762-3769; Hayes, C. S., and Sauer, R. T., Mol. Cell. 12 (2003) 903-911; Keiler, K. C., et al., Science 271 (1996) 990-993). With regard to a ribosome display selection, this mechanism lowers the amount of functional ternary complexes and the PCR-product yield is significantly reduced (Hanes, J., and Pluckthun, A., PNAS 94 (1997) 4937-4942).

This tmRNA induced ribosome rescue mechanism can be bypassed, when the ribosome translation machinery has been forced to stall before the 3′-end of the mRNA was encountered by the ribosome. Due to the induced translation arrest the ribosome A-site is still occupied. The display spacer of the ribosome display construct has the sequence as denoted in SEQ ID NO:62. With this spacer the translation can be arrested after the full polypeptide is translated and before the ribosome rescue mechanism is set off.

This has been achieved by removing translation stop codons (Mattheakis, L. C., et al., PNAS 91 (1994) 9022-9026; Hanes, J., and Pluckthun, A., PNAS 94 (1997) 4937-4942) from the DNA spacer sequence of the ribosome display construct. As a consequence a high molecular weight complex consisting of mRNA, the ribosome and the translationally stalled polypeptide is generated.

For the generation of libraries numerous techniques are known to a person skilled in the art. An exemplified proceeding is outlined below.

Linear Expression Elements (LEE) as basis of a DNA-library were produced in a modular manner. To rapidly support the overlapping extension ligation PCR (OEL-PCR) with the randomized DNA-fragments, a library of DNA-modules was pre-produced. In order to obtain sufficient PCR-product yield it was a prerequisite to use HPLC purified primer oligonucleotides and a DNA polymerase with a 3′-5′ exonucleolytic activity, producing blunt-end DNA fragments (Garrity, P. A., and Wold, B. J., PNAS 89 (1992) 1021-1025).

Exemplarily, the genes encoding the proteins PEX2 (c-terminal hemopexin-like domain of human matrix metalloproteinase 2), TIMP2 (tissue inhibitor of human matrix metalloproteinase 2), HDAC-I (human histone deacylase I), BirA (E. coli biotin holoenzyme ligase) and GFP (green fluorescent protein) were fused to different combinations of DNA-modules. The concentration of the PCR-products was determined by a comparative densitometric quantification using the LUMI Imager System (Roche Applied Sciences, Mannheim, Germany). The average PCR-product yield of the obtained Linear Expression Elements was about 60 ng/μl±20 ng/μl (ng per μl of PCR-mixture). Using the P. woesii DNA polymerase (PWO) it was possible to generate LEEs up to 2000 bp in length.

In an example a small library in which 8 amino acid positions of the PEX2 polypeptide were randomized was generated. For this purpose these positions and accordingly the following amino acids were chosen from the list for SEQ ID NO:10 as listed in table V: 528 (Gln), 529 (Glu), 550 (Arg), 576 (Lys), 577 (Asn), 578 (Lys), 594 (Val) and 596 (Lys). The library was generated by template free PCR synthesis as described in example 2. A ribosome display template was assembled e.g. by the modules T7P-g10epsilon-ATG (SEQ ID NO:74), a polypeptide from the generated library and a ribosome display spacer (SEQ ID NO:62).

A prerequisite for a suitable protein scaffold is its capability to stably fold in its active conformation, even under conditions where it has to carry the burden of multiple substituted amino acids. This can be examined by targeting the library versus a known protein-binding partner. In an example the PEX2 library was displayed to recognize the tissue inhibitor of metalloproteinase 2 (TIMP2) protein ligand. The randomized polypeptides from the PEX2-library were still able to recognize their inherent TIMP2 binding partner in a ribosome display approach. This indicated that the structure-function of the scaffold was maintained despite that the scaffold was multiply mutated.

To prepare and optimize the binding properties of a specific binder based on a polypeptide scaffold to a predetermined target molecule, which is not inherently bound by the scaffold, a cycle comprising four main steps has to be passed through several times. These steps are (i) alteration of at least one amino acid position according to table V, (ii) preparation of the display construct, (iii) display and selection of a specific binding variant and (iv) isolation and sequencing of the selected variant. Generally between two and five cycles are necessary to establish new specific binding characteristics in a scaffold.

The predetermined target molecule is not limited to a specific group of polypeptides. The predetermined polypeptide can belong e.g. to one of the groups of hedgehog proteins, bone morphogenetic proteins, growth factors, erythropoietin, thrombopoietin, G-CSF, interleukins and interferons, as well as to the groups of immunoglobulins, enzymes, inhibitors, activators, and cell surface proteins.

In an example, the non-PEX2 binder IGF-I was chosen as predetermined target molecule, for the generation of a specific binder, based on the PEX2 scaffold. The target molecule was plate-presented as a biotinylated ligand. After the second cycle of ribosome display with the PEX2 library a visible PCR-product signal was retained. This shows that the library is well suited for the selection of proteins/polypeptides specifically binding a predetermined target molecule not inherently bound by the protein/polypeptide.

The following examples, references and sequence listings are provided to aid the understanding of the present invention, the true scope of which is set forth in the appended claims. It is understood that modifications can be made in the procedures set forth without departing from the spirit of the invention.

### EXAMPLES

### Example 1

### Overlapping Extension Ligation PCR (OEL-PCR)

Linear Expression Elements were modularly assembled by a two step-PCR protocol, using the overlapping DNA ligation principle. In a standard PWO-PCR an intron-less open reading frame was amplified by sequence-specific terminal bridging primers, which generated overlapping homologous sequences to flanking DNA sequences. Two μl of the first PCR mixture containing approximately 50 ng of the elongated gene-fragment (gene-module) were transferred into a second PWO-PCR mixture. The mixture was supplied with 50 ng to 100 ng of pre-produced DNA-fragments (promotor- and terminator-module) and respective sequence specific, terminal primers at 1 μM each. Typically, this second PCR-step was comprised 30 cycles. The physical parameters of the PCR profiles were adjusted according to the requirements of the DNA-fragments to be ligated.

### Example 2

### Synthesis of the PEX2 DNA Library

The PEX2 triplet codons coding for the amino acid coordinates of the hemopexin-like domain 64, 65, 86, 112, 113, 114, 130 and 132 (equal to 528 (Gln), 529 (Glu), 550 (Arg), 576 (Lys), 577 (Asn), 578 (Lys), 594 (Val) and 596 (Lys) in the full length human matrix metalloproteinase 2) were randomized by NNK-motives. The human wild-type PEX2 DNA sequence was divided up into three sequence sections. A standard PWO-PCR, which was supplied with 10 ng vector-template pIVEX2.1MCS PEX2 and the primers PEX2forw (SEQ ID NO:63) and PEXR4 (SEQ ID NO:64) at 1 μM each amplified the 1 bp-218 bp fragment. The 402 bp-605 bp fragment was amplified in a standard PWO-PCR with 10 ng vector-template pIVEX2.1MCS PEX2 and the primers PEXF4 (SEQ ID NO:65) and PEX2rev (SEQ ID NO:66) at 1 μM each. The sequence 196 bp-432 bp formed overlaps with the DNA fragments 1 bp-218 bp and 402 bp-605 bp and was synthesized by template-free PCR with the primers PEXF1 (SEQ ID NO:67) and PEXR1 (SEQ ID NO:68) at 1 μM each and PEXR3 (SEQ ID NO:69), PEXR2 (SEQ ID NO:70) and PEXF2 (SEQ ID NO:71) at 0.25 μM each. The PCR-profile was the same for all three PCRs: TIM (initial melting temperature): 1 min at 94° C., TM (melting temperature): 20 sec at 94° C., TA (annealing temperature): 30 sec at 60° C., TE (elongation temperature): 15 sec at 72° C., 25 cycles, TFE (final elongation temperature): 2 min at 72° C. The full length randomized PEX2 sequence (588 bp) was obtained when 70 ng of each DNA sequence-fragment was applied to a standard PWO-PCR with the bridging primers T7P_PEX2 (SEQ ID NO:72) and PEX2_RD (SEQ ID NO:73) at 1 μM each. The PCR-profile was: TIM: 1 min at 94° C., TM: 20 sec at 94° C., TA: 30 sec at 60° C., TE: 60 sec at 72° C., 25 cycles, TFE: 5 min at 72° C. The bridging primers introduced homologues DNA overlaps for an assembly of the PEX2 gene-library into a ribosome display template by OEL-PCR.

### Example 3

### Cell-Free Protein In Vitro Transcription and Translation

According to the instructions of the manufacturer, Linear Expression Elements were transcribed and translated in the RTS 100 HY E. coli System. Linear DNA template (100 ng-500 ng) were incubated at 30° C. Optionally 6 μl GroE-supplement (Roche) was added.

### Example 4

### Site-Specific Biotinylation of Fusion Proteins

The RTS 100 E. coli HY System was modified for the sequence specific, enzymatic biotinylation. Sixty μl RTS mixture were assembled according to the manufacturer's instructions. The mixture was supplemented with 2 μl stock-solution Complete EDTA-Free Protease Inhibitor, 2 μM d-(+)-biotin, 50 ng T7P_BirA_T7T Linear Expression Element (1405 bp), coding for the E. coli Biotin Ligase (BirA, EC 6.3.4.15) and 100 ng to 500 ng linear template coding for the substrate fusion-protein. The substrate fusion-protein was N- or C-terminally fused to a Biotin Accepting Peptide sequence (BAP). In all experiments a 15-mer variant of sequence #85 as identified by Schatz (Schatz, P. J., Biotechnology (NY) 11 (1993) 1138-1143; Beckett, D. et al., Protein Sci. 8 (1999) 921-929) was used (Avitag, Avidity Inc., Denver, Colo. USA). Biotin Ligase was co-expressed from the linear template T7Pg10epsilon_birA_T7T.

### Example 5

### a) Ribosome Display Protocol

All buffers were kept on ice. All devices were sterile, Dnase- and Rnase-free. The workbench was cleaned with Rnase-ZAP.
10× Stock washing buffer (Stock WB) Ribosome Display: 0.5 M TRIS (tris(hydroxymethyl)-aminomethan), pH 7.5 (4° C.) adjusted by AcOH (acetic acid); 1.5 M NaCl; 0.5 M magnesium acetate, store at −20° C.
10× Elution buffer (Stock EB) Ribosome Display: 0.5 M TRIS, pH 7.5 (4° C.) adjusted by AcOH; 1.5 M NaCl; 200 mM EDTA, store at −20° C.
10 ml Ribosome Display Washing buffer (WB): 1200 μl 10× Stock WB pH 7.5, 0.05% TWEEN 20 (50 μL 10% TWEEN 20), 5% BSA (5 ml Blocker BSA 10%), 5 μg/ml t-RNA, 670 mM KCl (0.5 g KCl) ad. 10 ml with PCR-grade water
10 ml Ribosome Display Stopbuffer (SB): 1200 μL 10× Stock WB pH 7.5, 0.05% TWEEN 20 (50 μL 10% TWEEN 20), 5% BSA (5 ml Blocker BSA 10%), 5 μg/ml t-RNA, 670 mM KCl (0.5 g KCl), 4 mM GSSG (oxidized glutathione), 25 μM cAMP (10 μl Stock solution), ad. 10 ml with PCR-grade water
2 ml Ribosome Display Elution buffer: 200 μL 10× Stock EB, 0.25% BSA (50 μl Blocker BSA 10%), 5000 A260 units r-RNA 16S-23S ribosomal, 5 μg/ml t-RNA, ad. 2 ml with PCR-grade water
Blocking Reagent: 5% BSA Puffer (2.5 ml Blocker BSA 10%), 50% Conjugate Buffer Universal
10×PBS-buffer: 0.1 M NaH₂PO₄; 0.01 M KH₂PO₄ (10×pH 7.0; 1×pH 7.4); 1.37 M NaCl; 27 mM KCl.
   b) Preparation of the Ectodomains erbB2 and erbB3

The human receptor ectodomains erbB2 and erbB3 were obtained from R&D Systems as receptor chimeras. The receptor ectodomains were genetically fused to the human protein IgG1FC (human IgG1 antibody FC fragment). Both molecules revealed a molecular mass of 96 kDa and contained a hexahistidine-peptide at their C-terminus. As a result of glycosylation the molecular weight of the proteins was increased to 130 to 140 kDa. The chimeric proteins were obtained as lyophilized proteins and were resolubilized in PBS buffer containing 0.1% BSA. The proteins were stored at −80° C. until use.

### c) Coating of Micro Titer Plates

One Reaction Volume (RV) of a micro titer (MT)-plate was washed three times with Conjugate Buffer Universal. Two and a half (2.5) μg ligand was resolved in 100 μl Blocking Reagent. Biotinylated ligands were alternately immobilized in the wells of Streptavidin- and Avidin-coated MT-plates. The erbB2/FC- and erbB3/FC-chimeras were immobilized alternately in the wells of protein A and protein G coated MT-plates. The ligand-solution was incubated for 1 h at room temperature in the MT-plate under 500 rpm shaking on a Biorobot 8000 robotic shaker platform. To determine the background-signal a well was coated with 100 μl Blocking Reagent without ligand. The wells were washed with 3 RV Blocking Reagent. Blocking Reagent (300 μl) was incubated in each well for 1 h at 4° C. and 200 rpm. Before the stopped translation-mixture was applied, the wells were washed with 3 RV ice-cold buffer WB.

### d) Generation of Ribosome Display Templates

For the standard ribosome display procedure a single gene or a gene-library was elongated with specific bridging primers. The elongated DNA-fragments were fused by OEL-PCR to the DNA-modules T7Pg10epsilon (SEQ ID NO:74) and to the ribosome display spacer (SEQ ID NO:62) using the terminal primers T7Pfor (SEQ ID NO:75) and R1A (SEQ ID NO:76) 5′-AAATCGAAAGGCCCAGTTTTTCG-3′. The PCR profile for the PCR assembly was: TIM: 1 min at 94° C., TM: 20 sec at 94° C., TA: 30 sec at 60° C., TE: 60 sec for 1000 bp at 72° C., 30 cycles, TFE: 5 min at 72° C.

Production of the linear expression element (LEE) T7PAviTagFXa-PEX2-T7T: The human PEX2-gene was amplified in a standard PWO-PCR from 10 ng plasmid template pDSPEX2 (Roche) using the bridging primer according to SEQ ID NO:77 and to SEQ ID NO:78. The overlapping gene was fused by an OEL-PCR to the DNA-modules T7PAviTagFXa (SEQ ID NO:79) and T7T (SEQ ID NO:80) using the primers T7Pfor (SEQ ID NO:82) and T7Trev (SEQ ID NO:81).

### e) Preparation of the Ribosome Display Translation Mixture

The RTS E. coli 100 HY System was prepared according to the manufacture's instructions. One hundred μl of the mixture were supplemented with 40 units (1 μl) Rnasin, 2 μM (2 μl) anti ssrA-oligonucleotide 5′-TTAAGCTGCTAAAGCGTAGTTTTCGTCGTTTGCGACTA-3′ (SEQ ID NO:85), 1 μL stock solution of Complete Mini Protease Inhibitor EDTA-free and 500 ng linear ribosome display DNA-template in 20 μl PWO-PCR mixture. The ribosome display DNA-template was transcribed and translated in 1.5 ml reaction tubes at 30° C. for 40 min under shaking at 550 rpm. Complexes consisting of mRNA, ribosome and displayed polypeptide were stabilized when the reaction was immediately stopped with 500 μl ice-cold buffer SB. The mixture was centrifuged at 15.000 g at 2° C. for 10 min. The supernatant was transferred into a fresh, ice-cooled 1.5 ml reaction tube. Two hundred fifty μl of the mixture were transferred into a ligand-coated MT-plate well (signal) and another 250 μl into a non-ligand coated well (background). The mixture was incubated for 1 h at 4° C. and 300 rpm. To remove background protein and weak binding ternary complexes the wells were washed with ice-cold buffer WB. Messenger RNA from the bound ternary complexes was eluted by 100 μl ice-cold buffer EB for 10 min at 4° C. and 750 rpm.

### f) Preparation of Protein G Coated Magnetic Beads

Protein G coated magnetic beads were used to deplete the stopped ribosome display translation mixtures from protein derivatives, which unspecifically recognized IgG1-FC binders. One hundred μl of the magnetic bead suspension was equilibrated in stopping buffer SB by washing the beads five times in 500 μl buffer SB. The beads were incubated for 1 h at 4° C. in 500 μl buffer SB containing 50 μg IgG1-FC protein. The beads were washed three times with buffer SB and were stored on ice in 100 μl buffer SB. Prior to their use the beads were magnetically separated and stored on ice. The stopped ribosome display translation mixture was added to the beads. The mixture was incubated for 30 min at 4° C. at 750 rpm. Prior to use the beads were magnetically separated form the mixture.

g) Purification of mRNA and Removal of Remaining DNA

Messenger RNA was purified using the High Pure RNA Isolation Kit (Roche Applied Science, Mannheim, Germany). Remaining DNA-template in the eluate was removed with a modified protocol of the Ambion DNA-free kit (ambion Inc., USA). Fifty μl eluate were supplemented with 5.7 μl DNAse I buffer and 1.3 μl DNAse I containing solution. After incubation of the mixture at 37° C. for 30 min 6.5 μl DNAse I inactivating reagent was added. The slurry was incubated in the digestion-assay for 3 min at room temperature followed by 1 min centrifugation at 11,000 g. The supernatant was used in the reverse transcription

h) Reverse Transcription and cDNA Amplification

For the reverse transcription of the mRNA the C. therm. RT Polymerase Kit (Roche Applied Sciences, Mannheim, Germany) was used. Twenty μl reactions were assembled: 4 μl 5×RT buffer, 1 μl DTT (dithiothreitol) solution, 1.6 μl dNTP's, 1 μl DMSO solution, 0.1 μM (1 μl) RT 5′-CAGAGCCTGCACCAGCTCCAGAGCCAGC-3′ (SEQ ID NO:86), 40 units (1 μl) Rnasin, 1.5 μl C. therm. RNA-Polymerase, 9 μl mRNA containing eluate. Transcription was performed for 35 min at 70° C. Further amplification of the cDNA was performed in 100 μl PWO-PCRs containing 10 μl 10×PWO-PCR buffer with MgSO₄, 200 μM dNTPs, 12 μl transcription mixture, 2.5 units PWO DNA-Polymerase and the primers RT 5′-CAGAGCCTGCACCAGCTCCAGAGCCAGC-3′ (SEQ ID NO:86) and F1 5′-GTTTAACTTTAAGAAGGAGATATACATATG-3′ (SEQ ID NO:87) at 1 μM each. The PCR profile was TIM: 1 min at 94° C., TM: 20 sec at 94° C., TA: 30 sec at 60° C., TE: 60 sec at 72° C., 20 cycles, TFE: 5 min at 72° C. A reamplification by a standard PWO-PCR was performed. Two μl of the PCR mixture were transferred into a second standard PWO-PCR. Gene-specific bridging primers were used wherever possible. The PCR-profiles were according to the physical parameters of the gene-templates and oligonucleotide-primers. Twenty five PCR cycles were performed. The gene-sequences were elongated with DNA overlaps to hybridize with the DNA-modules T7Pg10epsilon and the ribosome display spacer in a further OEL-PCR. The ribosome display DNA-templates were then reused in further ribosome display cycles.

### i) Sub Cloning of Genes After Ribosome Display

The PCR-products were sub cloned into vector-systems with techniques know to a person skilled in the art. Library members of PEX2 were sub cloned via the NdeI/EcoRI sites into the vector pUC18 using the primers NdeI-PEX2for (SEQ ID NO:83) and EcoRI-PEX2rev (SEQ ID NO:84).

### LIST OF REFERENCES

Abo, T., et al. EMBO J. 19 (2000) 3762-3769
Altruda, F. et al., Nucleic Acids Res. 13 (1985) 3841-3859
Andersson, M., et al., J. Immunol. Methods 283 (2003) 225-234
Ausubel, I., and Frederick, M., Curr. Prot. Mol. Biol. (1992) John Wiley and Sons, New York
Beckett, D. et al., Protein Sci. 8 (1999) 921-929
Binz, H. K., et al., J. Mol. Biol. 332 (2003) 489-503
Bode, W., Structure 3 (1995) 527-530
Brooks, P. C., et al., Cell 92 (1998) 391-400
Faber, H. R., et al., Structure 3 (1995) 551-559
Forrer, P., et al., ChemBioChem 5 (2004) 183-189
Fulop, V., and Jones, D. T., Curr. Opin. Struct. Biol. 9 (1999) 715-721
Garrity, P. A., and Wold, B. J., PNAS 89 (1992) 1021-1025
Gohlke, U., et al., FEBS Lett. 378 (1996) 126-130
Gomis-Ruth, F. X., et al., J. Mol. Biol. 264 (1996) 556-566
Hames, B. D., and Higgins, S. G., Nucleic acid hybridization—a practical approach (1985) IRL Press, Oxford, England
Hanes, J., and Pluckthun, A., PNAS 94 (1997) 4937-4942
Hayes, C. S., and Sauer, R. T., Mol. Cell. 12 (2003) 903-911
He, M., and Taussig, M. J., Nuc. Acids Res. 25 (1997) 5132-5134
Ho, S. N., et al., Gene 77 (1989) 51-59
Jenne, D., and Stanley, K. K., Biochemistry 26 (1987) 6735-6742
Jenne, D., Biochem. Biophys. Res. Commun. 176 (1991) 1000-1006
Kain, K. C., et al., Biotechniques 10 (1991) 366-374
Keiler, K. C., et al., Science 271 (1996) 990-993
Klevenz, B., et al., Cell. Mol. Life. Sci. 59 (2002) 1993-1998
Lamla, T., and Erdmann, V. A., J. Mol. Biol. 329 (2003) 381-388
Lee, S. S., and Kang, C., Kor. Biochem. J. 24 (1991) 673-679
Letunic, I., et al., Nuc. Acids Res. 30 (2002) 242-244
Li, J., et al., Structure 3 (1995) 541-549
Libson, A. M., et al., Nat. Struct. Biol. 2 (1995) 938-942
Lu, Z., et al., Bio/Technology 13 (1995) 366-372
Mattheakis, L. C., et al., PNAS 91 (1994) 9022-9026
McConnell, S. J., and Hoess, R. H., J. Mol. Biol. 250 (1995) 460-470
Nygren, P.-A., and Skerra, A., J. Immun. Methods 290 (2004) 3-28
Predki, P. F., et al., Nat. Struct. Biol. 3 (1996) 54-58
Roberts, B. L., et al., Gene 121 (1992) 9-15
Roberts, B. L., et al., Proc. Natl. Acad. Sci. USA 89 (1992) 2429-2433
Rottgen, P., and Collins, J., Gene 164 (1995) 243-250
Sambrook, J., et al., Molecular Cloning: A laboratory manual (1999) Cold Spring Harbor Laboratory Press, New York, USA
Sandstrom, K., et al., Protein Eng. 16 (2003) 691-697
Schatz, P. J., Biotechnology (NY) 11 (1993) 1138-1143
Schultz, J., et al., PNAS 95 (1998) 5857-5864
Segal, D. J., et al., Biochemistry 42 (2003) 2137-2148
Shuldiner, A. R., et al., Anal. Biochem. 194 (1991) 9-15
Skerra, A., J. Mol. Recognit. 13 (2000) 167-187
Smith, G. P., et al., J. Mol. Biol. 277 (1998) 317-332
Smith, G. P., Science 228 (1985) 1315-1317
Stahl, S., and Uhlen, M., TIBTECH 15 (1997) 185-192
Sykes, K. F., and Johnston, S. A., Nature Biotechnol. 17 (1999) 355-359
Visintin, M., et al., J. Immun. Methods 290 (2004) 135-153
Wallon, U. M., and Overall, C. M., J. Biol. Chem. 272 (1997) 7473-7481
Willenbrock, F., et al., Biochemistry 32 (1993) 4330-4337 WO 01/04144
Xu, L., et al., Chem. Biol. 9 (2002) 933-942

1. A polypeptide that specifically binds a predetermined target molecule, wherein the amino acid sequence of said polypeptide is selected from the group consisting of SEQ ID NO:02 to SEQ ID NO:61, and wherein further at least one amino acid, of said amino acid sequence, according to table V is altered.

2. A process for the production of the polypeptide of claim 1 in a prokaryotic or eukaryotic microorganism, wherein said microorganism contains a nucleic acid sequence which encodes said polypeptide and said polypeptide is expressed.

3. The process of claim 2, wherein the polypeptide is isolated form the organism and purified.

4. The process of claim 2, wherein said predetermined target molecule is a member of one of the groups consisting of hedgehog proteins, bone morphogenetic proteins, growth factors, erythropoietin, thrombopoietin, G-CSF, interleukins and interferons.

5. A method for identifying a nucleic acid encoding a polypeptide which specifically binds a predetermined target molecule form a DNA-library, wherein said method comprises the steps of
a) selecting a sequence form the group consisting of SEQ ID NO:02 to SEQ ID NO:61;
b) preparing a DNA-library of the selected sequence in which at least one amino acid position according to table V is altered;
c) screening the prepared DNA-library for encoded polypeptides specifically binding a predetermined target molecule;
d) choosing the nucleic acid encoding one specific binder identified in step c);
e) repeating the steps b) to d) for two to five times, and
f) isolating said nucleic acid encoding a polypeptide specifically binding a predetermined target molecule.

6. The method of claim 5, wherein the DNA-library comprises linear expression elements.

7. The method as claimed in of claim 6, wherein the members of the library of the polypeptide are expressed by display on ribosomes.

8. The method of claim 6, wherein the members of the library of the polypeptide are expressed by display on bacteriophages.

9. The method for the determination of alterable amino acid positions in a polypeptide comprising the steps of
a) assembling of a plurality of sequences of polypeptides which are homologous in structure and/or function form the same and/or different organisms; and
b) aligning the sequences according to a common structural and/or consensus sequence and/or functional motif; and
c) determining the variability for all amino acids positions by counting the number of different amino acids found for each position of the sequence; and
d) identifying alterable amino acid positions as amino acid positions with a total number of different amino acids of eight or more.

10. A vector which is suitable for the expression of a polypeptide in a prokaryotic or eukaryotic microorganism, wherein said vector encodes the polypeptide of claim 1.

11-18. (canceled)
